(19) United States
(12) Patent Application Publication
(10) Pub. No.: US 2025/0268166 A1
(43) Pub. Date: Aug. 28, 2025

(54) ALS-INHIBITOR HERBICIDE TOLERANT BETA VULGARIS HYBRIDS WITH INCREASED HETEROSIS

(71) Applicants: BAYER AKTIENGESELLSCHAFT, Leverkusen (DE); KWS SAAT SE & CO. KGAA, Einbeck (DE)

(72) Inventors: Maik GERTZ, Hannover (DE); Martin MESSERSCHMIDT, Einbeck (DE); Ottó TÖRJÉK, Einbeck (DE)

(21) Appl. No.: 18/687,799

(22) PCT Filed: Aug. 29, 2022
(86) PCT No.: PCT/EP2022/073934

§ 371 Date: Feb. 28, 2024

(30) Foreign Application Priority Data
Sep. 2, 2021 (NZ) 779716

(51) Int. Cl. A01H 6/02; A01H 1/00; A01H 1/02; A01H 1/04; A01N 61/00; A01P 13/02; C12N 15/11; C12Q 1/6895

(52) U.S. Cl. CPC A01H 6/024; A01H 1/021; A01H 1/045; A01H 1/123; A01N 61/00; A01P 13/02; C12N 15/11; C12Q 1/6895

(57) ABSTRACT

Provided are improved herbicide tolerant Beta vulgaris hybrid plants and parts thereof, particularly improved hybrid sugar beet or fodder beet plants, with increased yield performance, while maintaining optimal and agronomically relevant herbicide tolerance, wherein both parent plants are derived by introgression of the herbicide tolerance gene from a single herbicide resistance donor genotype. Further provided are Beta vulgaris parent plants useful for obtaining such hybrid plants, markers for identifying such improved herbicide tolerant Beta vulgaris plants, as well methods for obtaining and identifying such improved herbicide tolerant Beta vulgaris hybrid plants.

### CROSS REFERENCE TO RELATED APPLICATIONS

This application is a 371 National Stage application of International Patent Application No. PCT/EP2022/073934, filed Aug. 29, 2022, which claims the benefit of priority of New Zealand Patent Application No. 779716, filed Sep. 2, 2021, each of which is incorporated herein by reference in its entirety.

### INCORPORATION OF SEQUENCE LISTING

The sequence listing that is contained in the file named “BCS211026_PCT.xml” which contains 22 sequence entries, is 34 kilo bytes (measured in operating system MS-Windows), and which is filed herewith is incorporated herein by reference in its entirety.

### FIELD OF THE INVENTION

The present invention relates to herbicide tolerant Beta vulgaris plants, such as ALS inhibitor herbicide tolerant Beta vulgaris plants, particularly sugar beet plants or fodder beet plants, as well as to methods of controlling unwanted vegetation in areas of growing Beta vulgaris plants by applying ALS inhibitor herbicides to such plants.

In particular, the invention relates to hybrid Beta vulgaris plants comprising a herbicide tolerant ALS allele in homozygous form wherein the herbicide tolerant ALS allele has been introgressed into parent plants from the same donor plant, and wherein the hybrid plants exhibit a reduced linkage drag and/or inbreeding depression.

### BACKGROUND

Cultivated forms of Beta vulgaris (as defined in Ford-Lloyd (2005) Sources of genetic variation, Genus Beta. In: Biancardi E, Campbell L G, Skaracis G N, De Biaggi M (eds) Genetics and Breeding of Sugar Beet. Science Publishers, Enfield (NH), USA, pp 25-33) are important agricultural crops in temperate and subtropical regions. For example, about 20% of the world sugar production is based on sugar beet. Because beet seedlings and juvenile plants during their first 6-8 weeks of their life are susceptible to strong competition caused by fast growing weeds, which outcompete the young crop plants, reliable weed control measures are imperative in these crop areas.

Herbicides are convenient tools to control weeds in cultured beets. The products used for this purpose, like phenmedipham, desmediphan and metamitron allow to suppress the growth of weeds in beet fields without damaging the crop. Nevertheless under adverse environmental conditions the efficacy of these products leaves room for improvements, especially if noxious weeds like Chenopodium album, Amaranthus retroflexus, Fallopia convolvulus and/or Tripleurospermum inodorata germinate over an extended period of time.

ALS inhibitor herbicides are widely used in modern agriculture due to their effectiveness at moderate application rates and relative non-toxicity in animals. By inhibiting ALS activity, these families of herbicides prevent further growth and development of susceptible plants including many weed species. In order to provide plants with an increased tolerance to even high concentrations of ALS inhibitor herbicides that may be required for sufficient weed control, additional ALS-inhibiting herbicide-resistant breeding lines and varieties of crop plants, as well as methods and compositions for the production and use of ALS inhibiting herbicide-resistant breeding lines and varieties, have been developed.

These ALS inhibitor herbicides inhibit the enzyme “acetohydroxyacid synthase” (AHAS), also known as “acetolactate synthase” (ALS [EC 4.1.3.18]). ALS is the site of action of five structurally diverse herbicide families belonging to the class of ALS inhibitor herbicides, like (a) sulfonylurea herbicides (Beyer E. M et al. (1988), Sulfonylureas in Herbicides: Chemistry, Degradation, and Mode of Action; Marcel Dekker, New York, 1988, 117-189), (b) sulfonylaminocarbonyltriazolinone herbicides (Pontzen, R., Pflanz.-Nachrichten Bayer, 2002, 55, 37-52), (c) imidazolinone herbicides (Shaner, D. L., et al., Plant Physiol., 1984, 76, 545-546; Shaner, D. L., and O'Connor, S. L. (Eds.) The Imidazolinone Herbicides, CRC Press, Boca Raton, FL, 1991), (d) triazolopyrimidine herbicides (Kleschick, W. A. et al., Agric. Food Chem., 1992, 40, 1083-1085), and (e) pyrimidinyl(thio)benzoate herbicides (Shimizu, T. J., Pestic. Sci., 1997, 22, 245-256; Shimizu, T. et al., Acetolactate Syntehase Inhibitors in Herbicide Classes in Development, Böger, P., Wakabayashi, K., Hirai, K., (Eds.), Springer Verlag, Berlin, 2002, 1-41).

ALS is involved in the conversion of two pyruvate molecules to an acetolactate molecule and carbon dioxide. The reaction uses thyamine pyrophosphate in order to link the two pyruvate molecules. The resulting product of this reaction, acetolactate, eventually becomes valine, leucine and isoleucine (Singh (1999) “Biosynthesis of valine, leucine and isoleucine”, in Plant Amino Acids, Singh, B. K., ed., Marcel Dekker Inc. New York, New York, pp. 227-247).

Inhibitors of ALS interrupt the biosynthesis of valine, leucine and isoleucine in plants. The consequence is an immediate depletion of the respective amino acid pools causing a stop of protein biosynthesis leading to a cessation of plant growth and eventually the plant dies, or—at least—is damaged.

Single base pair substitutions at specific sites of the ALS gene can lead to more or less resistant ALS enzyme variants which show different levels of inhibition by the ALS inhibitor herbicides. Plants comprising mutant ALS alleles therefore show different levels of tolerance to ALS inhibitor herbicides, depending on the chemical structure of the ALS inhibitor herbicide and the site of the point mutation in the ALS gene.

WO2012049268A1 describes the identification and isolation in Beta vulgaris of an ALS variant allele comprising a substitution in codon at nucleotide positions 1705-1707 of the endogenous ALS gene thereby encoding an ALS polypeptide having leucine instead of the naturally occurring tryptophan at amino acid position 569 (hereinafter also referred to as BvALS_W569L gene or allele). Beta vulgaris plants comprising this allele exhibit a strong and agronomically relevant tolerance to various ALS inhibitor herbicides as described also in WO2012049266A1.

Commercial Beta vulgaris seed, particularly sugar beet seed or fodder beet seed, is sold as hybrid seeds obtained by crossing (inbred) parent plants differing in genetic make-up, and harvesting the resulting seeds. Plants grown from such hybrid seeds are more vigorous and have higher yields than the respective parent plants, a phenomenon known as heterosis. Heterosis is due to a high degree of heterozygosity in the genome, that is, the presence of different alleles at many gene loci in the homologous chromosomes. To optimize the heterosis effect, the parent plants are selected from different so-called heterotic pools or groups, i.e. germplasm groups that are genetically distinct from each other.

For optimal ALS inhibitor herbicide tolerance, it is preferred that the ALS inhibitor herbicide tolerance conferring allele is present homozygously in Beta vulgaris plant, i.e. that each of the chromosomes 5 of diploid Beta vulgaris comprises the herbicide tolerance ALS gene, such as the BvALS_W569L allele.

To achieve homozygosity of herbicide tolerance ALS gene, such as the BvALS_W569L allele, in the hybrid Beta vulgaris plants, each parent plant needs to comprise at least one copy of the herbicide tolerant ALS gene, and, for optimal hybrid seed production, each parent plant preferably comprises the herbicide tolerant ALS gene also in homozygous form.

The isolation of non-transgenic Beta vulgaris plants comprising a herbicide tolerant ALS allele is a random and resource intensive process. Thus, rather than re-iterating the selection process for the mutant ALS allele in each heterotic pool, once an initial Beta vulgaris plant comprising such a mutant ALS allele is identified, this plant is used as donor plant to introgress the herbicide tolerance allele into the various heterotic pools. In Beta vulgaris, the ALS gene is located at chromosome 5, near the centromere. The introgression of the herbicide tolerant ALS allele thus leads to simultaneous introduction of large fragments of chromosome 5 from the donor plant linked to the herbicide tolerant ALS allele into each of the heterotic pools. Upon production of a homozygous hybrid plant, each of the chromosomes 5 thereof will consequently contain a large fragment of the chromosome 5 of the same donor plant, which are identical over at least a large part thereof, leading to a lower heterosis. This phenomenon is known in breeding as inbred or inbreeding depression.

In addition, the selection of ALS mutant alleles requires Beta vulgaris genotypes amenable to in vitro cell cultures, as described e.g. in WO2012049268A1. Such cell culture amenable genotypes are not (elite) genotypes, which are commercially relevant in various geographies, and usually contain unfavorable alleles present in the introgressed large chromosome 5 fragments. This phenomenon is known in breeding as linkage drag.

Thus, there remains a need for Beta vulgaris plants, such as sugar beet plants or fodder beet plants, comprising an ALS herbicide tolerance allele, such as BvALS_W569L, which can be combined to yield improved non-transgenic hybrid Beta vulgaris plants with increased heterosis, as expressed inter alia by increased yield, such as increased sugar yield, while maintaining optimal and agronomically relevant ALS herbicide tolerance. This problem is solved as described hereinafter in the description, drawings and claims.

### SUMMARY OF THE INVENTION

In a first aspect, the invention provides a Beta vulgaris hybrid plant or a part thereof, or hybrid seed or a part thereof, comprising an ALS-herbicide tolerant endogenous ALS gene allele which is homozygously present on chromosome 5 in said Beta vulgaris hybrid plant or seed, said ALS-herbicide tolerant endogenous ALS gene allele encoding an ALS protein comprising leucine at amino acid position 569, said Beta vulgaris hybrid plant or seed being obtainable by crossing two parent Beta vulgaris plants from different heterotic pools, wherein each parent plant comprises the ALS-herbicide tolerant endogenous ALS gene allele in homozygous state and wherein the ALS-herbicide tolerant endogenous ALS gene allele in each parent plant is introgressed from the same ALS-herbicide tolerant endogenous ALS gene allele donor plant, characterized in that chromosomal region of chromosome 5 introgressed from said ALS-herbicide tolerant endogenous ALS gene allele donor plant and located upstream and/or downstream of the ALS inhibitor tolerant endogenous ALS gene allele in said parent plant is sufficiently small to avoid or decrease inbred depression and/or increase heterosis and/or increase sugar yield or biomass yield in said Beta vulgaris hybrid plant. Preferably, the chromosomal region of chromosome 5 introgressed from said ALS-herbicide tolerant endogenous ALS gene allele donor plant and comprising said ALS-herbicide tolerant endogenous ALS gene allele is located (only or exclusively) on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M3 (comprising a nucleotide sequence of SEQ ID NO. 3), marker M4 (comprising a nucleotide sequence of SEQ ID NO. 4), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22) in one of the parent plants and in the other parent plant, said chromosomal region of chromosome 5 introgressed from said ALS-herbicide tolerant endogenous ALS gene allele donor plant and comprising said ALS-herbicide tolerant endogenous ALS gene allele is located (only or exclusively) on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22). The chromosomal region of chromosome 5 introgressed from said ALS-herbicide tolerant endogenous ALS gene allele donor plant and comprising said ALS-herbicide tolerant endogenous ALS gene allele can be located on a chromosomal interval as present in one or more seeds deposited with the NCIMB, Aberdeen, UK, under Number NCIMB 43836, NCIMB 43837 or NCIMB 43838 on Aug. 4, 2021, in one of the parent plants and in the other parent plant, whereby said chromosomal region of chromosome 5 introgressed from said ALS-herbicide tolerant endogenous ALS gene allele donor plant and comprising said ALS-herbicide tolerant endogenous ALS gene allele is located on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22). The chromosomal region of chromosome 5 introgressed from said ALS-herbicide tolerant endogenous ALS gene allele donor plant and comprising said ALS-herbicide tolerant endogenous ALS gene allele can also located on a chromosomal interval as present in one or more seeds deposited with the NCIMB, Aberdeen, UK, under Number NCIMB 43836, NCIMB 43837 or NCIMB 43838 on Aug. 4, 2021, in both of the parent plants.

In a further aspect of the invention, the invention provides seed as deposited with the NCIMB, Aberdeen, UK, under Number NCIMB 43836, NCIMB 43837 or NCIMB 43838 on Aug. 4, 2021, or a plant derived therefrom or grown out of this seed.

In another aspect of the invention, a DNA molecule is provided consisting of the chromosomal region of chromosome 5 of a Beta vulgaris ALS inhibitor herbicide tolerant plant comprising an ALS-herbicide tolerant endogenous ALS gene, reference seed of which has been deposited as NCIMB 41705, which is located on a chromosomal interval flanked on one side of the ALS-herbicide tolerant endogenous ALS gene allele by a marker selected from the group consisting of marker M1, marker M2, marker M3, marker M11, marker M15, marker M14, marker M4 and marker M5, and on the other side of the ALS-herbicide tolerant endogenous ALS gene allele is located on a chromosomal interval flanked by a marker selected from the group consisting of marker M10, marker M9, marker M8, marker M7, marker 13, marker 17, marker 16, marker M6 and marker M12. Preferably, said chromosomal region of chromosome 5 of a Beta vulgaris ALS inhibitor herbicide tolerant plant corresponds to the chromosomal region as present in a seed as deposited with the NCIMB, Aberdeen, UK, under Number NCIMB 43836, NCIMB 43837 or NCIMB 43838 on Aug. 4, 2021.

In yet another aspect of the invention, a Beta vulgaris plant or seed or a part thereof is provided comprising on one or both chromosomes 5 endogenously a DNA molecule as herein described.

Also provided by the invention is the use of a hybrid Beta vulgaris plant as herein described for the production of sugar, ethanol, biogas, betaine and/or uridine or for production of animal feed.

The invention further provides a method for identifying a genomic fragment of chromosome 5 in a(n) (elite) Beta vulgaris plant, introgressed from an ALS-herbicide tolerant Beta vulgaris donor plant, said genomic fragment comprising an ALS-herbicide tolerant endogenous ALS gene allele, or for identifying/selecting a Beta vulgaris plant comprising said genomic fragment of chromosome 5, comprising the steps of identifying the presence of the ALS-herbicide tolerant endogenous ALS gene allele in said plant by a phenotypic or a marker-assisted method; and identifying the presence or absence of at least one allele/nucleotide on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22); and optionally selecting an ALS inhibitor tolerant Beta vulgaris plant, wherein said plant exhibits a decreased inbred depression and/or an increased heterosis and/or an increased sugar yield or biomass yield.

The presence of the at least one allele/nucleotide can be identified in a(n) (elite) Beta vulgaris plant using one or more markers selected from the group of marker 1 (SEQ ID NO. 1), marker 2 (SEQ ID NO. 2), marker 3 (SEQ ID NO. 3), marker 4 (SEQ ID NO. 4), marker 5 (SEQ ID NO. 5), marker 6 (SEQ ID NO. 6), marker 7 (SEQ ID NO. 7), marker 8 (SEQ ID NO. 8), marker 9 (SEQ ID NO. 9), marker 10 (SEQ ID NO. 10), marker 11 (SEQ ID NO. 11), marker 12 (SEQ ID NO. 12), marker 13 (SEQ ID NO. 13), marker 14 (SEQ ID NO. 19), marker 15 (SEQ ID NO. 20), marker 16 (SEQ ID NO. 21) and marker 17 (SEQ ID NO. 22). Alternatively, the method for identifying a genomic fragment of chromosome 5 in (elite) Beta vulgaris plants, introgressed from an ALS-herbicide tolerant Beta vulgaris donor plant, said genomic fragment comprising an ALS-herbicide tolerant endogenous ALS gene allele, comprises the steps of identifying the presence of at least one first allele/nucleotide on a chromosomal interval flanked by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and by the ALS-herbicide tolerant endogenous ALS gene allele, and identifying the presence of at least one second allele/nucleotide on a chromosomal interval flanked by the ALS-herbicide tolerant endogenous ALS gene allele and by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22). Preferably, the presence of the at least one first allele/nucleotide is identified by one or more markers selected from the group of marker 1 (SEQ ID NO. 1), marker 2 (SEQ ID NO. 2), marker 3 (SEQ ID NO. 3), marker 4 (SEQ ID NO. 4), marker 5 (SEQ ID NO. 5), marker 11 (SEQ ID NO. 11), marker 14 (SEQ ID NO. 19) and marker 15 (SEQ ID NO. 20), and the at least one second allele/nucleotide is identified by one or more markers selected from the group of marker 10 (SEQ ID NO. 10), marker 9 (SEQ ID NO. 9), marker 8 (SEQ ID NO. 8), marker 7 (SEQ ID NO. 7), marker 13 (SEQ ID NO. 13), marker 17 (SEQ ID NO. 22), marker 16 (SEQ ID NO. 21) and marker 6 (SEQ ID NO. 6).

The invention further provides marker(s) as herein described i.e. DNA molecules comprising the nucleotide sequence of any one of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 19, SEQ ID NO. 20, SEQ ID NO. 21 or SEQ ID NO. 22. The invention further encompasses the use of said marker(s) for the identification or selection of

The invention additionally provides a method for decreasing inbred depression and/or increasing heterosis and/or increasing sugar yield or biomass yield in an ALS herbicide tolerant Beta vulgaris hybrid plant comprising an ALS-herbicide tolerant endogenous ALS gene allele which is homozygously present on chromosome 5 in said Beta vulgaris hybrid plant, said ALS herbicide tolerant endogenous ALS gene allele encoding an ALS protein comprising leucine at position 569, said method comprises crossing two parent Beta vulgaris plants from different heterotic pools, wherein each parent plant comprises the ALS herbicide tolerant endogenous ALS gene allele in homozygous state and wherein the ALS-herbicide tolerant endogenous ALS gene allele in each parent plant is introgressed from the same ALS-herbicide tolerant endogenous ALS gene allele donor plant, characterized in that the chromosomal region of chromosome 5 introgressed from said ALS-herbicide tolerant endogenous ALS gene allele donor plant and comprising said ALS-herbicide tolerant endogenous ALS gene allele is located on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M3 (comprising a nucleotide sequence of SEQ ID NO. 3), marker M4 (comprising a nucleotide sequence of SEQ ID NO. 4), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22) in one of the parent plants and in the other parent plant, said chromosomal region of chromosome 5 introgressed from said ALS-herbicide tolerant endogenous ALS gene allele donor plant and comprising said ALS-herbicide tolerant endogenous ALS gene allele is located on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22). The chromosomal region of chromosome 5 introgressed from said ALS-herbicide tolerant endogenous ALS gene allele donor plant and comprising said ALS-herbicide tolerant endogenous ALS gene allele can be located on a chromosomal interval as present in one or more seeds deposited with the NCIMB, Aberdeen, UK, under Number NCIMB 43836, NCIMB 43837 or NCIMB 43838 on Aug. 4, 2021, in one of the parent plants and in the other parent plant, whereby said chromosomal region of chromosome 5 introgressed from said ALS-herbicide tolerant endogenous ALS gene allele donor plant and comprising said ALS-herbicide tolerant endogenous ALS gene allele is located on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22). The chromosomal region of chromosome 5 introgressed from said ALS-herbicide tolerant endogenous ALS gene allele donor plant and comprising said ALS-herbicide tolerant endogenous ALS gene allele can be located on a chromosomal interval as present in one or more seeds deposited with the NCIMB, Aberdeen, UK, under Number NCIMB 43836, NCIMB 43837 or NCIMB 43838 on Aug. 4, 2021, in both of the parent plants.

It is another aspect of the invention to provide the use of one or more ALS inhibitor herbicide(s) for controlling unwanted vegetation in Beta vulgaris growing areas wherein the Beta vulgaris plants are hybrid Beta vulgaris plants as herein described. The ALS inhibitor herbicide(s) may be applied in combination with non-ALS inhibitor herbicides (i.e. herbicides showing a mode of action that is different to the inhibition of the ALS enzyme [acetohydroxyacid synthase; EC 2.2.1.6] group D herbicides), and wherein the non-ALS inhibitor herbicide(s) is/are selected form the group consisting of chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, glyphosate, glyphosate-isopropylammonium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl, sethoxydim.

Further provided by the invention is a method for controlling unwanted vegetation in Beta vulgaris plant growing areas, characterized by (a) the presence of Beta vulgaris plants as herein described; (b) the application of one or more ALS inhibitor herbicide(s) alone or in combination with one or more herbicide(s) that do(es) not belong to the class of ALS inhibitor herbicides (non-ALS inhibitor herbicides), wherein the application of the respective herbicides as defined under (b) can (i) take place jointly or simultaneously, or (ii) take place at different times and/or in a plurality of portions (sequential application), in pre-emergence applications followed by post-emergence applications or early post-emergence applications followed by medium or late post-emergence applications.

Embodiments of the invention are reflected in the following numbered paragraphs:

Embodiment 1. A Beta vulgaris hybrid plant, or hybrid seed, or a part thereof comprising an ALS-herbicide tolerant endogenous ALS gene allele which is homozygously present on chromosome 5 in said Beta vulgaris hybrid plant or seed, said ALS-herbicide tolerant endogenous ALS gene allele encoding an ALS protein comprising leucine at position 569, said Beta vulgaris hybrid plant or seed being obtainable by crossing two parent Beta vulgaris plants, preferably from different heterotic pools, wherein each parent plant comprises the ALS-herbicide tolerant endogenous ALS gene allele in homozygous state and wherein the ALS-herbicide tolerant endogenous ALS gene allele in each parent plant is introgressed from the same ALS-herbicide tolerant endogenous ALS gene allele donor plant, characterized in that chromosomal region of chromosome 5 introgressed from said ALS-herbicide tolerant endogenous ALS gene allele donor plant and located upstream and/or downstream of the ALS inhibitor tolerant endogenous ALS gene allele in said parent plant is sufficiently small to avoid or decrease inbred depression and/or increase heterosis and/or increase sugar yield in said Beta vulgaris hybrid plant.

Embodiment 2. The Beta vulgaris hybrid plant, or hybrid seed, or a part thereof of embodiment 1, wherein the ALS-herbicide tolerant endogenous ALS gene allele encodes an ALS protein comprising (i) an amino acid sequence which is at least 80%, 85% or 90%, at least 91,%, 92%, 93%, 94% or 95%, or at least 96%, 97%, 98% or 99% identical to the sequence as set forth in SEQ ID NO. 18, wherein the amino acid corresponding to the amino acid at position 569 of SEQ ID NO. 18 is leucine instead of tryptophane, or (ii) the amino acid of SEQ ID NO: 16.

Embodiment 3. The Beta vulgaris hybrid plant, or hybrid seed, or a part thereof of embodiment 1 or 2, wherein the ALS-herbicide tolerant endogenous ALS gene allele comprises (i) the nucleotide sequence of SEQ ID NO. 15, or (ii) the nucleotide sequence which is degenerated as a result of the genetic code to the nucleotide sequence of (i).

Embodiment 4. The Beta vulgaris hybrid plant, or hybrid seed, or a part thereof of any one of embodiments 1 to 3, wherein the chromosomal region of chromosome 5 introgressed from said ALS-herbicide tolerant endogenous ALS gene allele donor plant and comprising said ALS-herbicide tolerant endogenous ALS gene allele is located (only or exclusively) on a chromosomal interval flanked on one side of the ALS-herbicide tolerant endogenous ALS gene allele by a marker selected from the group consisting of marker M1, marker M2, marker M3, marker M4, marker M5, marker M11, marker M14 and marker M15 and on the other side of the ALS-herbicide tolerant endogenous ALS gene allele is flanked by a marker selected from the group consisting of marker M6, marker M12, marker M16, marker M17, marker M13, marker M7, marker M8, marker M9 and marker M10 in one of the parent plants and in the other parent plant, said chromosomal region of chromosome 5 introgressed from said ALS-herbicide tolerant endogenous ALS gene allele donor plant and comprising said ALS-herbicide tolerant endogenous ALS gene allele is located (only or exclusively) on a chromosomal interval flanked on one side of the ALS-herbicide tolerant endogenous ALS gene allele by a marker selected from the group consisting of marker M1, marker M2, marker M3, marker M4, marker M5, marker M11, marker M14 and marker M15, and on the other side of the ALS-herbicide tolerant endogenous ALS gene allele is flanked by a marker selected from the group consisting of marker M6, marker M12, marker M16, marker M17, marker M13, marker M7, marker M8, marker M9 and marker M10.

Embodiment 5. The Beta vulgaris hybrid plant, or hybrid seed, or a part thereof of anyone of embodiments 1 to 4, wherein the chromosomal region introgressed from said ALS-herbicide tolerant endogenous ALS gene allele donor plant of chromosome 5 and comprising said ALS-herbicide tolerant endogenous ALS gene allele on one of the chromosomes 5 is located (only or exclusively) on a chromosomal interval which
a. is flanked on one side of the ALS-herbicide tolerant endogenous ALS gene allele by marker M1 and on the other side of the ALS-herbicide tolerant endogenous ALS gene allele by marker M6;
b. is flanked on one side of the ALS-herbicide tolerant endogenous ALS gene allele by marker M1 and on the other side of the ALS-herbicide tolerant endogenous ALS gene allele by marker M7;
c. is flanked on one side of the ALS-herbicide tolerant endogenous ALS gene allele by marker M1 and on the other side of the ALS-herbicide tolerant endogenous ALS gene allele by marker M8;
d. is flanked on one side of the ALS-herbicide tolerant endogenous ALS gene allele by marker M1 and on the other side of the ALS-herbicide tolerant endogenous ALS gene allele by marker M9;
e. is flanked on one side of the ALS-herbicide tolerant endogenous ALS gene allele by marker M1 and on the other side of the ALS-herbicide tolerant endogenous ALS gene allele by marker M10;
f. is flanked on one side of the ALS-herbicide tolerant endogenous ALS gene allele by marker M2; and on the other side of the ALS-herbicide tolerant endogenous ALS gene allele by marker M6;
g. is flanked on one side of the ALS-herbicide tolerant endogenous ALS gene allele by marker M2 and on the other side of the ALS-herbicide tolerant endogenous ALS gene allele by marker M7;
h. is flanked on one side of the ALS-herbicide tolerant endogenous ALS gene allele by marker M2 and on the other side of the ALS-herbicide tolerant endogenous ALS gene allele by marker M8;
i. is flanked on one side of the ALS-herbicide tolerant endogenous ALS gene allele by marker M2 and on the other side of the ALS-herbicide tolerant endogenous ALS gene allele by marker M9;
j. is flanked on one side of the ALS-herbicide tolerant endogenous ALS gene allele by marker M2 and on the other side of the ALS-herbicide tolerant endogenous ALS gene allele by marker M10;
k. is flanked on one side of the ALS-herbicide tolerant endogenous ALS gene allele by marker M3 and on the other side of the ALS-herbicide tolerant endogenous ALS gene allele by marker M6;
l. is flanked on one side of the ALS-herbicide tolerant endogenous ALS gene allele by marker M3 and on the other side of the ALS-herbicide tolerant endogenous ALS gene allele by marker M7;
m. is flanked on one side of the ALS-herbicide tolerant endogenous ALS gene allele by marker M3 and on the other side of the ALS-herbicide tolerant endogenous ALS gene allele by marker M8;
n. is flanked on one side of the ALS-herbicide tolerant endogenous ALS gene allele by marker M3 and on the other side of the ALS-herbicide tolerant endogenous ALS gene allele by marker M9;
o. is flanked on one side of the ALS-herbicide tolerant endogenous ALS gene allele by marker M3 and on the other side of the ALS-herbicide tolerant endogenous ALS gene allele by marker M10;
p. is flanked on one side of the ALS-herbicide tolerant endogenous ALS gene allele by marker M4 and on the other side of the ALS-herbicide tolerant endogenous ALS gene allele by marker M6;
q. is flanked on one side of the ALS-herbicide tolerant endogenous ALS gene allele by marker M4 and on the other side of the ALS-herbicide tolerant endogenous ALS gene allele by marker M7;
r. is flanked on one side of the ALS-herbicide tolerant endogenous ALS gene allele by marker M4 and on the other side of the ALS-herbicide tolerant endogenous ALS gene allele by marker M8;
s. is flanked on one side of the ALS-herbicide tolerant endogenous ALS gene allele by marker M4 and on the other side of the ALS-herbicide tolerant endogenous ALS gene allele by marker M9;
t. is flanked on one side of the ALS-herbicide tolerant endogenous ALS gene allele by marker M4 and on the other side of the ALS-herbicide tolerant endogenous ALS gene allele by marker M10;
u. is flanked on one side of the ALS-herbicide tolerant endogenous ALS gene allele by marker M5 and on the other side of the ALS-herbicide tolerant endogenous ALS gene allele by marker M6;
v. is flanked on one side of the ALS-herbicide tolerant endogenous ALS gene allele by marker M5 and on the other side of the ALS-herbicide tolerant endogenous ALS gene allele by marker M7;
w. is flanked on one side of the ALS-herbicide tolerant endogenous ALS gene allele by marker M5 and on the other side of the ALS-herbicide tolerant endogenous ALS gene allele by marker M8;
x. is flanked on one side of the ALS-herbicide tolerant endogenous ALS gene allele by marker M5 and on the other side of the ALS-herbicide tolerant endogenous ALS gene allele by marker M9; or
y. is flanked on one side of the ALS-herbicide tolerant endogenous ALS gene allele by marker M5 and on the other side of the ALS-herbicide tolerant endogenous ALS gene allele by marker M10.

Embodiment 6. The Beta vulgaris hybrid plant, or hybrid seed, or a part thereof of any one of embodiments 1 to 5, wherein the chromosomal region introgressed from said ALS-herbicide tolerant endogenous ALS gene allele donor plant of chromosome 5 and comprising said ALS-herbicide tolerant endogenous ALS gene allele on one of the chromosomes 5 is located (only or exclusively) on a chromosomal interval which
a. is flanked on one side of the ALS-herbicide tolerant endogenous ALS gene allele by marker M1 and on the other side of the ALS-herbicide tolerant endogenous ALS gene allele by marker M12;
b. is flanked on one side of the ALS-herbicide tolerant endogenous ALS gene allele by marker M1 and on the other side of the ALS-herbicide tolerant endogenous ALS gene allele by marker M13;
c. is flanked on one side of the ALS-herbicide tolerant endogenous ALS gene allele by marker M1 and on the other side of the ALS-herbicide tolerant endogenous ALS gene allele by marker M10;
d. is flanked on one side of the ALS-herbicide tolerant endogenous ALS gene allele by marker M2 and on the other side of the ALS-herbicide tolerant endogenous ALS gene allele by marker M12; or,
e. is flanked on one side of the ALS-herbicide tolerant endogenous ALS gene allele by marker M2 and on the other side of the ALS-herbicide tolerant endogenous ALS gene allele by marker M13;
f. is flanked on one side of the ALS-herbicide tolerant endogenous ALS gene allele by marker M2 and on the other side of the ALS-herbicide tolerant endogenous ALS gene allele by marker M10;
g. is flanked on one side of the ALS-herbicide tolerant endogenous ALS gene allele by marker M5 and on the other side of the ALS-herbicide tolerant endogenous ALS gene allele by marker M12;
h. is flanked on one side of the ALS-herbicide tolerant endogenous ALS gene allele by marker M5 and on the other side of the ALS-herbicide tolerant endogenous ALS gene allele by marker M13;
i. is flanked on one side of the ALS-herbicide tolerant endogenous ALS gene allele by marker M5 and on the other side of the ALS-herbicide tolerant endogenous ALS gene allele by marker M10;
j. is flanked on one side of the ALS-herbicide tolerant endogenous ALS gene allele by marker M11 and on the other side of the ALS-herbicide tolerant endogenous ALS gene allele by marker M12;
k. is flanked on one side of the ALS-herbicide tolerant endogenous ALS gene allele by marker M11 and on the other side of the ALS-herbicide tolerant endogenous ALS gene allele by marker M13; or
l. is flanked on one side of the ALS-herbicide tolerant endogenous ALS gene allele by marker M11 and on the other side of the ALS-herbicide tolerant endogenous ALS gene allele by marker M10.

Embodiment 7. The Beta vulgaris hybrid plant, or hybrid seed, or a part thereof of any one of embodiments 1 to 6, wherein the chromosomal region introgressed from said ALS-herbicide tolerant endogenous ALS gene allele donor plant of chromosome 5 and comprising said ALS-herbicide tolerant endogenous ALS gene allele on one of the chromosomes 5 is located (only or exclusively) on a chromosomal interval which
a. is flanked on one side of the ALS-herbicide tolerant endogenous ALS gene allele by marker M5 and on the other side of the ALS-herbicide tolerant endogenous ALS gene allele by marker M16;
b. is flanked on one side of the ALS-herbicide tolerant endogenous ALS gene allele by marker M5 and on the other side of the ALS-herbicide tolerant endogenous ALS gene allele by marker M17;
c. is flanked on one side of the ALS-herbicide tolerant endogenous ALS gene allele by marker M14 and on the other side of the ALS-herbicide tolerant endogenous ALS gene allele by marker M16;
d. is flanked on one side of the ALS-herbicide tolerant endogenous ALS gene allele by marker M14 and on the other side of the ALS-herbicide tolerant endogenous ALS gene allele by marker M17;
e. is flanked on one side of the ALS-herbicide tolerant endogenous ALS gene allele by marker M15 and on the other side of the ALS-herbicide tolerant endogenous ALS gene allele by marker M16;
f. is flanked on one side of the ALS-herbicide tolerant endogenous ALS gene allele by marker M15 and on the other side of the ALS-herbicide tolerant endogenous ALS gene allele by marker M17.

Embodiment 8. The Beta vulgaris hybrid plant, or hybrid seed, or a part thereof of any one of embodiments 1 to 7, wherein the chromosomal regions introgressed from said ALS-herbicide tolerant endogenous ALS gene allele donor plant and comprising said ALS-herbicide tolerant endogenous ALS gene allele of chromosome 5 as derived from parent plants are the same.

Embodiment 9. The Beta vulgaris hybrid plant, or hybrid seed, or a part thereof of any one of embodiments 1 to 7, wherein the chromosomal region introgressed from said ALS-herbicide tolerant endogenous ALS gene allele donor plant and comprising said ALS-herbicide tolerant endogenous ALS gene allele of chromosome 5 is located (only or exclusively) on a chromosomal interval which
a. is flanked by marker M1 and marker M10 on both of the chromosomes 5;
b. is flanked by marker M1 and marker M9 on one of the chromosomes 5 and by marker M1 and marker M10 on the other chromosome 5;
c. is flanked by marker M1 and marker M8 on one of the chromosomes 5 and by marker M1 and marker M10 on the other chromosome 5;
d. is flanked by marker M2 and marker M8 on one of the chromosomes 5 and by marker M2 and M13 on the other chromosome 5;
e. is flanked by marker M3 and marker M7 on one of the chromosomes 5 and by marker M11 and M13 on the other chromosome 5;
f. is flanked by marker M4 and marker M7 on one of the chromosomes 5 and by marker M11 and M13 on the other chromosome 5; or
g. is flanked by marker M5 and marker M6 on one of the chromosomes 5 and by marker M5 and M12 on the other chromosome 5.

Embodiment 10. The Beta vulgaris hybrid plant, or hybrid seed, or a part thereof of any one of embodiments 1 to 9, wherein the chromosomal region introgressed from said ALS-herbicide tolerant endogenous ALS gene allele donor plant of chromosome 5 and comprising said ALS-herbicide tolerant endogenous ALS gene allele is located (only or exclusively) on a chromosomal interval flanked by marker M4 and marker M7 on one of the chromosomes 5 and by marker M11 and M13 on the other chromosome 5.

Embodiment 11. The Beta vulgaris hybrid plant, or hybrid seed, or a part thereof of any one of embodiments 1 to 9, wherein the chromosomal region introgressed from said ALS-herbicide tolerant endogenous ALS gene allele donor plant of chromosome 5 and comprising said ALS-herbicide tolerant endogenous ALS gene allele is located (only or exclusively) on a chromosomal interval flanked by marker M5 and marker M6 on one of the chromosomes 5 and by marker M5 and M12 on the other chromosome 5.

Embodiment 12. The Beta vulgaris hybrid plant, or hybrid seed, or a part thereof of any one of embodiments 1 to 11 wherein the markers comprise a nucleotide at the varying position of the markers, as present in the genomic region of chromosome 5 of the parent plants.

Embodiment 13. The Beta vulgaris hybrid plant, or hybrid seed, or a part thereof of any one of embodiments 1 to 12 wherein said ALS-herbicide tolerant endogenous ALS gene allele encodes an ALS protein comprising leucine at position 569.

Embodiment 14. The Beta vulgaris hybrid plant, or hybrid seed, or a part thereof of any one of embodiments 1 to 12 wherein said ALS-herbicide tolerant endogenous ALS gene allele comprises marker Mals.

Embodiment 15. The Beta vulgaris hybrid plant, or hybrid seed, or a part thereof of any one of embodiments 1 to 14 comprising a nucleotide sequence having at least 90% sequence identity to the nucleotide sequence of SEQ ID NO. 15 or encoding an amino acid sequence having at least 90% sequence identity to the nucleotide sequence of SEQ ID NO. 16.

Embodiment 16. The Beta vulgaris hybrid plant, or hybrid seed, or a part thereof of any one of embodiments 1 to 15 wherein the ALS-herbicide tolerant endogenous ALS gene allele donor plant is a Beta vulgaris ALS inhibitor herbicide tolerant plant reference seed of which being deposited as NCIMB 41705.

Embodiment 17. The Beta vulgaris hybrid plant, or hybrid seed, or a part thereof of any one of embodiments 1 to 16 having a sugar yield of at least 80%, at least 85%, at least 90%, at least 91%, at least 92%, at least 93%, at least 94%, at least 95%, at least 96%, at least 97%, at least 98%, at least 99%, or equal to or greater to the sugar yield of an isogenic Beta vulgaris hybrid plant comprising a wild-type allele of the ALS encoding gene on chromosome 5 in homozygous state.

Embodiment 18. The Beta vulgaris hybrid plant, or hybrid seed, or a part thereof of embodiment 1, wherein the chromosomal region introgressed from said ALS-herbicide tolerant endogenous ALS gene allele donor plant of chromosome 5 and comprising said ALS-herbicide tolerant endogenous ALS gene allele is the chromosomal region as present in one or more seeds deposited with the NCIMB, Aberdeen, UK, under Number NCIMB 43836, NCIMB 43837 or NCIMB 43838 on Aug. 4, 2021.

Embodiment 19. The Beta vulgaris hybrid plant, or hybrid seed, or a part thereof of embodiment 1 grown out of or derived from a seed as deposited with the NCIMB, Aberdeen, UK, under Number NCIMB 43836, NCIMB 43837 or NCIMB 43838 on Aug. 4, 2021.

Embodiment 20. A DNA molecule consisting of the chromosomal region of chromosome 5 of a Beta vulgaris ALS inhibitor herbicide tolerant plant comprising an ALS-herbicide tolerant endogenous ALS gene, reference seed of which has been deposited as NCIMB 41705, which is located (only or exclusively) on a chromosomal interval flanked on one side of the ALS-herbicide tolerant endogenous ALS gene allele by a marker selected from the group consisting of marker M1, marker M2, marker M3, marker M4, marker M11, marker M5, marker M14 and marker M15, and on the other side of the ALS-herbicide tolerant endogenous ALS gene allele is flanked by a marker selected from the group consisting of marker M6, marker M7, marker M8, marker M9 and marker M10, marker M12, marker M13, marker M16 and marker M17.

Embodiment 21. The DNA molecule of embodiment 20, wherein said chromosomal region is located (only or exclusively) on a chromosomal interval flanked by
a. markers M1 and M6;
b. markers M1 and M7;
c. markers M1 and M8;
d. markers M1 and M9;
e. markers M1 and M10;
f. markers M2 and M6;
g. markers M2 and M7;
h. markers M2 and M8;
i. markers M2 and M9;
j. markers M2 and M10;
k. markers M3 and M6;
l. markers M3 and M7;
m. markers M3 and M8;
n. markers M3 and M9;
o. markers M3 and M10;
p. markers M4 and M6;
q. markers M4 and M7;
r. markers M4 and M8;
s. markers M4 and M9;
t. markers M4 and M10;
u. markers M5 and M6;
v. markers M5 and M7;
w. markers M5 and M8;
x. markers M5 and M9;
y. markers M5 and M10.
z. markers M1 and M12;
aa. markers M1 and M13;
bb. markers M1 and M10;
cc. markers M2 and M12;
dd. markers M2 and M13;
ee. markers M2 and M10;
ff. markers M5 and M12;
gg. markers M5 and M13;
hh. markers M5 and M10;
ii. markers M11 and M12;
jj. markers M11 and M13;
kk. markers M11 and M10;
ll. markers M5 and M16;
mm. markers M5 and M17;
nn. markers M14 and M16;
oo. markers M14 and M17;
pp. markers M15 and M16; or
qq. markers M15 and M17.

Embodiment 22. The DNA molecule of embodiment 20, wherein said chromosomal region is located (only or exclusively) on a chromosomal interval flanked by markers M5 and M6.

Embodiment 23. The DNA molecule of embodiment 20, wherein said chromosomal region is located (only or exclusively) on a chromosomal interval flanked by markers M4 and M7.

Embodiment 24. The DNA molecule of embodiment 20, wherein said chromosomal region is located (only or exclusively) on a chromosomal interval flanked by markers M5 and M12.

Embodiment 25. The DNA molecule of embodiment 20, wherein said chromosomal region is located (only or exclusively) on a chromosomal interval flanked by markers M11 and M13.

Embodiment 26. The DNA molecule of embodiment 20, wherein said chromosomal region is located (only or exclusively) on a chromosomal interval flanked by markers M5 and M16.

Embodiment 27. The DNA molecule of embodiment 20, wherein said chromosomal region is located (only or exclusively) on a chromosomal interval flanked by markers M14 and M17.

Embodiment 28. The DNA molecule of embodiment 20, wherein said chromosomal region is located (only or exclusively) on a chromosomal interval flanked by markers M15 and M17.

Embodiment 29. The DNA molecule of embodiment 20, wherein said chromosomal region is located (only or exclusively) on a chromosomal interval as present in one or more seeds deposited with the NCIMB, Aberdeen, UK, under Number NCIMB 43836, NCIMB 43837 or NCIMB 43838 on Aug. 4, 2021.

Embodiment 30. A Beta vulgaris plant or seed comprising on chromosome 5 endogenously a DNA molecule according to any one of embodiments 20 to 29.

Embodiment 31. A Beta vulgaris plant comprising on both chromosomes 5 endogenously a DNA molecule according to any one of embodiments 20 to 29.

Embodiment 32. A method of producing a hybrid Beta vulgaris seed comprising crossing a Beta vulgaris plant according to embodiment 30 or 31 with another Beta vulgaris plant according to embodiment 30 or 31 and harvesting the progeny seed.

Embodiment 33. Use of a hybrid Beta vulgaris plant according to any one of embodiments 1 to 19 for the production of sugar, ethanol, biogas, betaine and/or uridine or for the production of animal feed.

Embodiment 34. A method for identifying a genomic fragment of chromosome 5 in (elite) Beta vulgaris plants, introgressed from an ALS-herbicide tolerant Beta vulgaris donor plant, said genomic fragment comprising an ALS-herbicide tolerant endogenous ALS gene allele, comprising the steps of
a. Identifying the presence of the ALS-herbicide tolerant endogenous ALS gene allele in said plant by a phenotypic or a marker-assisted method; and
b. Identifying the presence or absence of at least one allele/nucleotide on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22).

Embodiment 35. A method for identifying a genomic fragment of chromosome 5 in Beta vulgaris plants, introgressed from an ALS-herbicide tolerant Beta vulgaris donor plant, said genomic fragment comprising an ALS-herbicide tolerant endogenous ALS gene allele, comprising the steps of
a. identifying the presence of the ALS-herbicide tolerant endogenous ALS gene allele in said plant by a phenotypic or a marker-assisted method; and
b. identifying the presence of the allele/nucleotide as present in the (elite) Beta vulgaris plant at nucleotide position 31 of one or more markers selected from the group of marker M1 (SEQ ID NO. 1), marker M2 (SEQ ID NO. 2), marker M3 (SEQ ID NO. 3), marker M4 (SEQ ID NO. 4), marker M5 (SEQ ID NO. 5), marker M6 (SEQ ID NO. 6), marker M7 (SEQ ID NO. 7), marker M8 (SEQ ID NO. 8), marker M9 (SEQ ID NO. 9), marker M10 (SEQ ID NO. 10), marker M11 (SEQ ID NO. 11) and marker 13 (SEQ ID NO. 13), at position 30 of marker M12 (SEQ ID NO. 12), or at position 101 of one or more markers selected from the group of marker M14 (SEQ ID NO. 19), marker M15 (SEQ ID NO. 20), marker M16 (SEQ ID NO. 21), marker M17 (SEQ ID NO. 22).

Embodiment 36. A method for identifying or selecting a Beta vulgaris plant comprising the genomic fragment of chromosome 5 as described in embodiments 34 or 35, comprising the steps of
a. identifying the presence of the ALS-herbicide tolerant endogenous ALS gene allele in said plant by a phenotypic or a marker-assisted method;
b. identifying the presence of at least one allele/nucleotide on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22); and
c. optionally selecting an ALS inhibitor tolerant Beta vulgaris plant, wherein said plant exhibits a decreased inbred depression and/or an increased heterosis and/or an increased sugar yield or biomass yield.

Embodiment 37. A method for identifying a genomic fragment of chromosome 5 in Beta vulgaris plants, introgressed from an ALS-herbicide tolerant Beta vulgaris donor plant, said genomic fragment comprising an ALS-herbicide tolerant endogenous ALS gene allele, comprising the steps of
a. identifying the presence of at least one first allele/nucleotide on a chromosomal interval flanked by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and by the ALS-herbicide tolerant endogenous ALS gene allele, and
b. identifying the presence of at least one second allele/nucleotide on a chromosomal interval flanked by the ALS-herbicide tolerant endogenous ALS gene allele and by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22).

Embodiment 38. A method for identifying or selecting a Beta vulgaris plant comprising the genomic fragment of chromosome 5 as described in embodiments 34 or 35, comprising the steps of
a. identifying the presence of at least one first allele/nucleotide on a chromosomal interval flanked by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and by the ALS-herbicide tolerant endogenous ALS gene allele,
b. identifying the presence of at least one second allele/nucleotide on a chromosomal interval flanked by the ALS-herbicide tolerant endogenous ALS gene allele and by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22); and optionally selecting an ALS inhibitor tolerant Beta vulgaris plant, wherein said plant exhibits a decreased inbred depression and/or an increased heterosis and/or an increased sugar yield or biomass yield.

Embodiment 39. The method for identifying/selection according to anyone of embodiments 34 to 38, wherein the presence of the at least one allele/nucleotide is identified by one or more markers selected from the group of marker 1 (SEQ ID NO. 1), marker 2 (SEQ ID NO. 2), marker 3 (SEQ ID NO. 3), marker 4 (SEQ ID NO. 4), marker 5 (SEQ ID NO. 5), marker 6 (SEQ ID NO. 6), marker 7 (SEQ ID NO. 7), marker 8 (SEQ ID NO. 8), marker 9 (SEQ ID NO. 9), marker 10 (SEQ ID NO. 10), marker 11 (SEQ ID NO. 11), marker 12 (SEQ ID NO. 12), marker 13 (SEQ ID NO. 13), marker 14 (SEQ ID NO. 19), marker 15 (SEQ ID NO. 20), marker 16 (SEQ ID NO. 21) and marker 17 (SEQ ID NO. 22).

Embodiment 40. The method of anyone of embodiment 34 to 39, wherein the step of identifying the presence of an allele/nucleotide comprises identifying
a. the presence of a G at nucleotide position 31 of marker M1 (SEQ ID NO. 1);
b. the presence of a T at nucleotide position 31 of marker M2 (SEQ ID NO. 2);
c. the presence of a G at nucleotide position 31 of marker M3 (SEQ ID NO. 3);
d. the presence of an A at nucleotide position 31 of marker M4 (SEQ ID NO. 4);
e. the presence of a C at nucleotide position 31 of marker M5 (SEQ ID NO. 5);
f. the presence of a C at nucleotide position 31 of marker M6 (SEQ ID NO. 6);
g. the presence of a C at nucleotide position 31 of marker M7 (SEQ ID NO. 7);
h. the presence of a C at nucleotide position 31 of marker M8 (SEQ ID NO. 8);
i. the presence of an A at nucleotide position 31 of marker M9 (SEQ ID NO. 9);
j. the presence of an A at nucleotide position 31 of marker M10 (SEQ ID NO. 10);
k. the presence of an A at nucleotide position 31 of marker M11 (SEQ ID NO. 11);
l. the presence of a T at nucleotide position 30 of marker M12 (SEQ ID NO. 12); or
m. the presence of an A at nucleotide position 31 of marker M13 (SEQ ID NO. 13);
n. the presence of an C at nucleotide position 101 of marker M14 (SEQ ID NO. 19);
o. the presence of an T at nucleotide position 101 of marker M15 (SEQ ID NO. 20);
p. the presence of an G at nucleotide position 101 of marker M16 (SEQ ID NO. 21); or
q. the presence of an C at nucleotide position 101 of marker M17 (SEQ ID NO. 22).

Embodiment 41. The method of anyone of embodiment 34 to 39, wherein the step of identifying the presence of an allele/nucleotide comprises identifying
a. the presence of an A at nucleotide position 31 of marker M4 (SEQ ID NO. 4) and the presence of a C at nucleotide position 31 of marker M7 (SEQ ID NO. 7);
b. the presence of an A at nucleotide position 31 of marker M11 (SEQ ID NO. 11) and the presence of an A at nucleotide position 31 of marker M13 (SEQ ID NO. 13;
c. the presence of a C at nucleotide position 31 of marker M5 (SEQ ID NO. 5) and the presence of a C at nucleotide position 31 of marker M6 (SEQ ID NO. 6); or
d. the presence of a C at nucleotide position 31 of marker M5 (SEQ ID NO. 5) and the presence of a T at nucleotide position 30 of marker M12 (SEQ ID NO. 12); or
e. the presence of a C at nucleotide position 31 of marker M5 (SEQ ID NO. 5) and the presence of a G at nucleotide position 101 of marker M16 (SEQ ID NO. 21); or
f. the presence of a C at nucleotide position 101 of marker M14 (SEQ ID NO. 19) and the presence of a C at nucleotide position 101 of marker M17 (SEQ ID NO. 22); or
g. the presence of a T at nucleotide position 101 of marker M15 (SEQ ID NO. 20) and the presence of a C at nucleotide position 101 of marker M17 (SEQ ID NO. 22).

Embodiment 42. The method of any one of embodiments 34 to 41, wherein the step of identifying the presence of the ALS-herbicide tolerant endogenous ALS gene allele comprises identifying the presence of a G nucleotide at nucleotide position 31 of marker Mals (SEQ ID NO. 14).

Embodiment 43. A method for decreasing inbred depression and/or increasing heterosis and/or increasing sugar yield or biomass yield in an ALS herbicide tolerant Beta vulgaris hybrid plant comprising an ALS-herbicide tolerant endogenous ALS gene allele which is homozygously present on chromosome 5 in said Beta vulgaris hybrid plant, said ALS herbicide tolerant endogenous ALS gene allele encoding an ALS protein comprising leucine at position 569, said method comprises crossing two parent Beta vulgaris plants, preferably from different heterotic pools, wherein each parent plant comprises the ALS herbicide tolerant endogenous ALS gene allele in homozygous state and wherein the ALS-herbicide tolerant endogenous ALS gene allele in each parent plant is introgressed from the same ALS-herbicide tolerant endogenous ALS gene allele donor plant, characterized in that the chromosomal region of chromosome 5 introgressed from said ALS-herbicide tolerant endogenous ALS gene allele donor plant and comprising said ALS-herbicide tolerant endogenous ALS gene allele is located on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M3 (comprising a nucleotide sequence of SEQ ID NO. 3), marker M4 (comprising a nucleotide sequence of SEQ ID NO. 4), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22) in one of the parent plants and in the other parent plant, said chromosomal region of chromosome 5 introgressed from said ALS-herbicide tolerant endogenous ALS gene allele donor plant and comprising said ALS-herbicide tolerant endogenous ALS gene allele is located on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22).

Embodiment 44. The method for decreasing inbred depression and/or increasing heterosis and/or increasing sugar yield or biomass yield in an ALS herbicide tolerant Beta vulgaris hybrid plant according to embodiment 43, wherein the chromosomal region of chromosome 5 introgressed from said ALS-herbicide tolerant endogenous ALS gene allele donor plant and comprising said ALS-herbicide tolerant endogenous ALS gene allele is located on a chromosomal interval as present in one or more seeds deposited with the NCIMB, Aberdeen, UK, under Number NCIMB 43836, NCIMB 43837 or NCIMB 43838 on Aug. 4, 2021, in one of the parent plants and/or in the other parent plant, wherein said chromosomal region of chromosome 5 introgressed from said ALS-herbicide tolerant endogenous ALS gene allele donor plant and comprising said ALS-herbicide tolerant endogenous ALS gene allele is located on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22).

Embodiment 45. The method for decreasing inbred depression and/or increasing heterosis and/or increasing sugar yield or biomass yield in an ALS herbicide tolerant Beta vulgaris hybrid plant according to embodiments 43 and 44, wherein the chromosomal region of chromosome 5 introgressed from said ALS-herbicide tolerant endogenous ALS gene allele donor plant and comprising said ALS-herbicide tolerant endogenous ALS gene allele is located on a chromosomal interval as present in one or more seeds deposited with the NCIMB, Aberdeen, UK, under Number NCIMB 43836, NCIMB 43837 or NCIMB 43838 on Aug. 4, 2021, in both of the parent plants.

Embodiment 46. A DNA molecule comprising the nucleotide sequence of any one of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 19, SEQ ID NO: 20, SEQ ID NO: 21 or SEQ ID NO: 22.

Embodiment 47. Use of one or more ALS inhibitor herbicide(s) for controlling unwanted vegetation in Beta vulgaris growing areas wherein the Beta vulgaris plants are hybrid Beta vulgaris plants according to claims 1 to 19.

Embodiment 48. Use of one or more ALS inhibitor herbicide(s) according to embodiment 47, wherein the ALS inhibitor herbicide(s) belong(s) to:
the group of the (sulfon)amides (group (A)) consisting of:
   the subgroup (A1) of the sulfonylureas, consisting of:
   amidosulfuron [CAS RN 120923-37-7](=A1-1);
   azimsulfuron [CAS RN 120162-55-2](=A1-2);
   bensulfuron-methyl [CAS RN 83055-99-6](=A1-3);
   chlorimuron-ethyl [CAS RN 90982-32-4](=A1-4);
   chlorsulfuron [CAS RN 64902-72-3](=A1-5);
   cinosulfuron [CAS RN 94593-91-6](=A1-6);
   cyclosulfamuron [CAS RN 136849-15-5](=A1-7);
   ethametsulfuron-methyl [CAS RN 97780-06-8](=A1-8);
   ethoxysulfuron [CAS RN 126801-58-9](=A1-9);
   flazasulfuron [CAS RN 104040-78-0](=A1-10);
   flucetosulfuron [CAS RN 412928-75-7](=A1-11);
   flupyrsulfuron-methyl-sodium [CAS RN 144740-54-5](=A1-12);
   foramsulfuron [CAS RN 173159-57-4](=A1-13);
   halosulfuron-methyl [CAS RN 100784-20-1](=A1-14);
   imazosulfuron [CAS RN 122548-33-8](=A1-15);
   iodosulfuron-methyl-sodium [CAS RN 144550-36-7](=A1-16);
   mesosulfuron-methyl [CAS RN 208465-21-8](=A1-17);
   metsulfuron-methyl [CAS RN 74223-64-6](=A1-18);
   monosulfuron [CAS RN 155860-63-2](=A1-19);
   nicosulfuron [CAS RN 111991-09-4](=A1-20);
   orthosulfamuron [CAS RN 213464-77-8](=A1-21);
   oxasulfuron [CAS RN 144651-06-9](=A1-22);
   primisulfuron-methyl [CAS RN 86209-51-0](=A1-23);
   prosulfuron [CAS RN 94125-34-5](=A1-24);
   pyrazosulfuron-ethyl [CAS RN 93697-74-6](=A1-25);
   rimsulfuron [CAS RN 122931-48-0](=A1-26);
   sulfometuron-methyl [CAS RN 74222-97-2](=A1-27);
   sulfosulfuron [CAS RN 141776-32-1](=A1-28);
   thifensulfuron-methyl [CAS RN 79277-27-3](=A1-29);
   triasulfuron [CAS RN 82097-50-5](=A1-30);
   tribenuron-methyl [CAS RN 101200-48-0](=A1-31);
   trifloxysulfuron [CAS RN 145099-21-4](sodium) (=A1-32);
   triflusulfuron-methyl [CAS RN 126535-15-7](=A1-33);
   tritosulfuron [CAS RN 142469-14-5](=A1-34);
   NC-330 [CAS RN 104770-29-8](=A1-35);
   NC-620 [CAS RN 868680-84-6](=A1-36);
   TH-547 [CAS RN 570415-88-2](=A1-37);
   monosulfuron-methyl [CAS RN 175076-90-1](=A1-38);
   2-iodo-N-[(4-methoxy-6-methyl-1,3,5-triazinyl)carbamoyl]benzene-sulfonamide (=A1-39);
   a compound of the general formula (I)

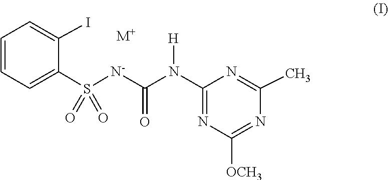

where M+ denotes the respective salt of the compound (I), i.e. its lithium salt (=A1-40); its sodium salt (=A1-41); its potassium salt (=A1-42); its magnesium salt (=A1-43); its calcium (=A1-44); its ammonium salt (=A1-45); its methylammonium salt (=A1-46); its dimethylammonium salt (=A1-47); its tetramethylammonium salt (=A1-48); its ethylammonium salt (=A1-49); its diethylammonium salt (=A1-50); its tetraethylammonium salt (=A1-51); its propylammonium salt (=A1-52); its tetrapropylammonium salt (=A1-53); its isopropylammonium salt (=A1-54); its diisopropylammonium salt (=A1-55); its butylammonium salt (=A1-56); its tetrabutylammonium salt (=A1-57); its (2-hydroxyeth-1-yl)ammonium salt (=A1-58); its bis-N,N-(2-hydroxyeth-1-yl)ammonium salt (=A1-59); its tris-N,N,N-(2-hydroxyeth-1-yl)ammonium salt (=A1-60); its 1-phenylethylammonium salt (=A1-61); its 2-phenylethylammonium salt (=A1-62); its trimethylsulfonium salt (=A1-63); its trimethyloxonium salt (=A1-64); its pyridinium salt (=A1-65); its 2-methylpyridinium salt (=A1-66); its 4-methylpyridinium salt (=A1-67); its 2,4-dimethylpyridinium salt (=A1-68); its 2,6-dimethylpyridinium salt (=A1-69); its piperidinium salt (=A1-70); its imidazolium salt (=A1-71); its morpholinium salt (=A1-72); its 1,5-diazabicyclo[4.3.0]non-7-enium salt (=A1-73); its 1,8-diazabicyclo[5.4.0]undec-7-enium salt (=A1-74);
or a compound of the formula (II) or salts thereof

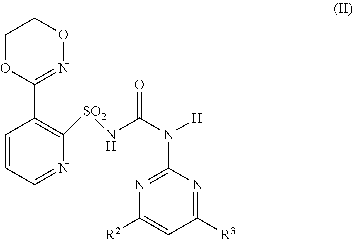

with R2, and R3 having the meaning as defined in the below table

| Compound | R² | R³ |
|---|---|---|
| A1-75 | OCH₃ | OC₂H₅ |
| A1-76 | OCH₃ | CH₃ |
| A1-77 | OCH₃ | C₂H₅ |
| A1-78 | OCH₃ | CF₃ |
| A1-79 | OCH₃ | OCF₂H |
| A1-80 | OCH₃ | NHCH₃ |
| A1-81 | OCH₃ | N(CH₃)₂ |
| A1-82 | OCH₃ | Cl |
| A1-83 | OCH₃ | OCH₃ |
| A1-84 | OC₂H₅ | OC₂H₅ |
| A1-85 | OC₂H₅ | CH₃ |
| A1-86 | OC₂H₅ | C₂H₅ |

or the compound of formula (III) (=A1-87), i.e. the sodium salt of compound (A1-83)

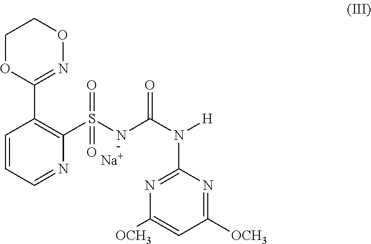 [SMILES: COc1cc(OC)nc(NC(=O)NS(=O)(=O)c2ncccc2C2=NOCCO2)n1 ; [Na+]]

or the compound of formula (IV) (=A1-88), i.e. the sodium salt of compound (A1-82)

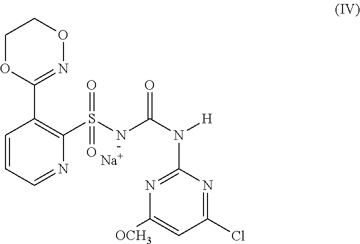 [SMILES: COc1cc(Cl)nc(NC(=O)NS(=O)(=O)c2ncccc2C2=NOCCO2)n1 ; [Na+]]

the subgroup of the sulfonylaminocarbonyltriazolinones (subgroup ((A2)), consisting of:
   flucarbazone-sodium [CAS RN 181274-17-9](=A2-1);
   propoxycarbazone-sodium [CAS RN 181274-15-7](=A2-2);
   thiencarbazone-methyl [CAS RN 317815-83-1](=A2-3);
the subgroup of the triazolopyrimidines (subgroup (A3)), consisting of:
   cloransulam-methyl [147150-35-4](=A3-1);
   diclosulam [CAS RN 145701-21-9](=A3-2);
   florasulam [CAS RN 145701-23-1](=A3-3);
   flumetsulam [CAS RN 98967-40-9](=A3-4);
   metosulam [CAS RN 139528-85-1](=A3-5);
   penoxsulam [CAS RN 219714-96-2](=A3-6);
   pyroxsulam [CAS RN 422556-08-9](=A3-7);
the subgroup of the sulfonanilides (subgroup (A4)), consisting of:
   compounds or salts thereof from the group described by the general formula (V):

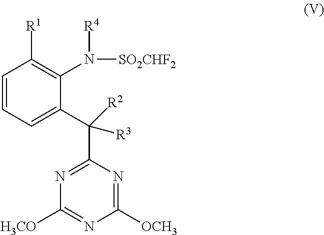

in which
R1 is halogen, preferably fluorine or chlorine,
R2 is hydrogen and R3 is hydroxyl or R2 and R3 together with the carbon atom to which they are attached are a carbonyl group C═O and
R4 is hydrogen or methyl;
and more especially compounds of the below given chemical structure (A4-1) to (A4-8)

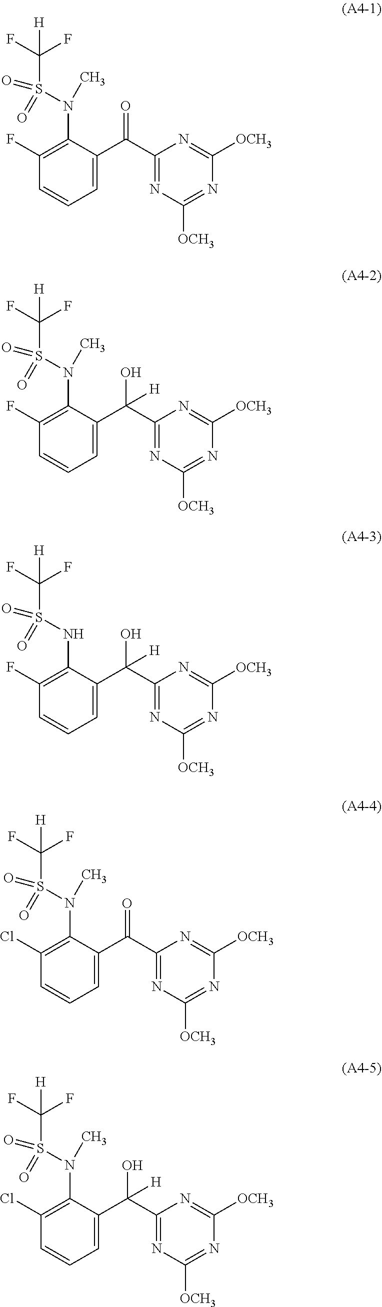 [SMILES: COc1nc(OC)nc(C(=O)c2cccc(F)c2N(C)S(=O)(=O)C(F)F)n1 ; COc1nc(OC)nc(C(O)c2cccc(F)c2N(C)S(=O)(=O)C(F)F)n1 ; COc1nc(OC)nc(C(O)c2cccc(F)c2NS(=O)(=O)C(F)F)n1 ; COc1nc(OC)nc(C(=O)c2cccc(Cl)c2N(C)S(=O)(=O)C(F)F)n1 ; COc1nc(OC)nc(C(O)c2cccc(Cl)c2N(C)S(=O)(=O)C(F)F)n1]

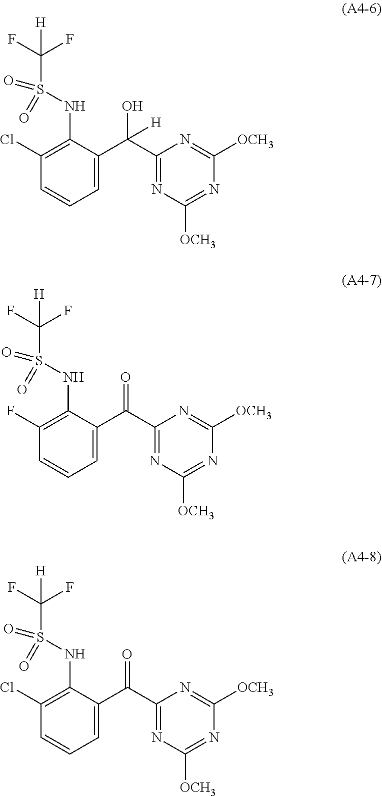 [SMILES: COc1nc(OC)nc(C(O)c2cccc(Cl)c2NS(=O)(=O)C(F)F)n1 ; COc1nc(OC)nc(C(=O)c2cccc(F)c2NS(=O)(=O)C(F)F)n1 ; COc1nc(OC)nc(C(=O)c2cccc(Cl)c2NS(=O)(=O)C(F)F)n1]

the group of the imidazolinones (group (B1)), consisting of:
   imazamethabenzmethyl [CAS RN 81405-85-8](=B1-1);
   imazamox [CAS RN 114311-32-9](=B1-2);
   imazapic [CAS RN 104098-48-8](=B1-3);
   imazapyr [CAS RN 81334-34-1](=B1-4);
   imazaquin [CAS RN 81335-37-7](=B1-5);
   imazethapyr [CAS RN 81335-77-5](=B1-6);
   SYP-298 [CAS RN 557064-77-4](=B1-7);
   SYP-300 [CAS RN 374718-10-2](=B1-8);
the group of the pyrimidinyl(thio)benzoates (group (C)), consisting of:
   the subgroup of the pyrimidinyloxybenzoeacids (subgroup (C1)) consisting of:
      bispyribac-sodium [CAS RN 125401-92-5](=C1-1);
      pyribenzoxim [CAS RN 168088-61-7](=C1-2);
      pyriminobac-methyl [CAS RN 136191-64-5](=C1-3);
      pyribambenz-isopropyl [CAS RN 420138-41-6](=C1-4);
      pyribambenz-propyl [CAS RN 420138-40-5](=C1-5);
   the subgroup of the pyrimidinylthiobenzoeacids (subgroup (C2)), consisting of:
      pyriftalid [CAS RN 135186-78-6](=C2-1);
      pyrithiobac-sodium [CAS RN 123343-16-8](=C2-2).

Embodiment 49. Use of one or more ALS inhibitor herbicide(s) according to embodiment 47 or 48, wherein the ALS inhibitor herbicide(s) belong(s) to the group consisting of:
amidosulfuron [CAS RN 120923-37-7](=A1-1);
chlorimuron-ethyl [CAS RN 90982-32-4](=A1-4);
ethametsulfuron-methyl [CAS RN 97780-06-8](=A1-8);
ethoxysulfuron [CAS RN 126801-58-9](=A1-9);
flupyrsulfuron-methyl-sodium [CAS RN 144740-54-5](=A1-12);
foramsulfuron [CAS RN 173159-57-4](=A1-13);
iodosulfuron-methyl-sodium [CAS RN 144550-36-7](=A1-16);
mesosulfuron-methyl [CAS RN 208465-21-8](=A1-17);
metsulfuron-methyl [CAS RN 74223-64-6](=A1-18);
monosulfuron [CAS RN 155860-63-2](=A1-19);
nicosulfuron [CAS RN 111991-09-4](=A1-20);
sulfosulfuron [CAS RN 141776-32-1](=A1-28);
thifensulfuron-methyl [CAS RN 79277-27-3](=A1-29);
tribenuron-methyl [CAS RN 101200-48-0](=A1-31);
2-iodo-N-[(4-methoxy-6-methyl-1,3,5-triazinyl)carbamoyl]benzene-sulfonamide (=A1-39);
2-iodo-N-[(4-methoxy-6-methyl-1,3,5-triazinyl)carbamoyl]benzene-sulfonamide sodium salt (=A1-41);
(A1-83) or its sodium salt (=A1-87);
propoxycarbazone-sodium [CAS RN 181274-15-7](=A2-2);
thiencarbazone-methyl [CAS RN 317815-83-1](=A2-3);
florasulam [CAS RN 145701-23-1](=A3-3);
metosulam [CAS RN 139528-85-1](=A3-5);
pyroxsulam [CAS RN 422556-08-9](=A3-7)
(A4-1);
(A4-2);
(A4-3);
imazamox [CAS RN 114311-32-9](=B1-2); and
bispyribac-sodium [CAS RN 125401-92-5](=C1-1).

Embodiment 50. Use of one or more ALS inhibitor herbicide(s) according to embodiment 47 or 48, wherein the ALS inhibitor herbicide(s) belong(s) to the group consisting of: amidosulfuron [CAS RN 120923-37-7](=A1-1);
foramsulfuron [CAS RN 173159-57-4](=A1-13);
iodosulfuron-methyl-sodium [CAS RN 144550-36-7](=A1-16);
2-iodo-N-[(4-methoxy-6-methyl-1,3,5-triazinyl)carbamoyl]benzene-sulfonamide (=A1-39);
2-iodo-N-[(4-methoxy-6-methyl-1,3,5-triazinyl)carbamoyl]benzene-sulfonamide sodium salt (=A1-41);
A1-83 or its sodium salt (=A1-87);
thiencarbazone-methyl [CAS RN 317815-83-1](=A2-3);
imazamox [CAS RN 114311-32-9](=B1-2);
bispyribac-sodium [CAS RN 125401-92-5](=C1-1).

Embodiment 51. Use of one or more ALS inhibitor herbicide(s) according to embodiment 47 or 48, wherein the ALS inhibitor herbicide(s) comprises foramsulfuron [CAS RN 173159-57-4](=A1-13) and thiencarbazone-methyl [CAS RN 317815-83-1](=A2-3).

Embodiment 52. Use of one or more ALS inhibitor herbicide(s) according to any of embodiments 47 to 51 in combination with non-ALS inhibitor herbicides (i.e. herbicides showing a mode of action that is different to the inhibition of the ALS enzyme [acetohydroxyacid synthase; EC 2.2.1.6] group D herbicides), and wherein the non-ALS inhibitor herbicide(s) is/are selected form the group consisting of:
chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, glyphosate, glyphosate-isopropylammonium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl, sethoxydim.

Embodiment 53. Use of one or more ALS inhibitor herbicide(s) according to embodiment 52, and wherein the non-ALS inhibitor herbicide(s) is/are selected form the group consisting of:
desmedipham, ethofumesate, glufosinate, glufosinate-ammonium, glufosinate-P, glufosinate-P-ammonium, glufosinate-P-sodium, glyphosate, glyphosate-isopropylammonium, lenacil, metamitron, phenmedipham, phenmedipham-ethyl.

Embodiment 54. Method for controlling unwanted vegetation in Beta vulgaris plant growing areas, characterized by:
(a) the presence of Beta vulgaris plants according to any one of embodiments 1 to 19
(b) the application of one or more ALS inhibitor herbicide(s) alone or in combination with one or more herbicide(s) that do(es) not belong to the class of ALS inhibitor herbicides (non-ALS inhibitor herbicides), and
(c) wherein the application of the respective herbicides as defined under (b)
   (i) takes place jointly or simultaneously, or
   (ii) takes place at different times and/or in a plurality of portions (sequential application), in pre-emergence applications followed by post-emergence applications or early post-emergence applications followed by medium or late post-emergence applications.

Embodiment 55. Method according to embodiment 54 for controlling unwanted vegetation, and wherein the ALS inhibitor herbicide(s) are taken from the groups as defined in anyone of embodiments 48 to 51.

Embodiment 56. Method according to embodiment 54 for controlling unwanted vegetation, and wherein the ALS inhibitor herbicide(s) comprise foramsulfuron [CAS RN 173159-57-4](=A1-13) and thiencarbazone-methyl [CAS RN 317815-83-1](=A2-3).

Embodiment 57. Method according to any one of embodiments 54 to 56, and wherein the non-ALS inhibitor herbicide(s) are taken from the group consisting of:
chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, glyphosate, glyphosate-isopropylammonium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl, sethoxydim.

### BRIEF DESCRIPTION OF THE DRAWINGS

FIG. 1: Schematic representation of the identification of homologous recombination events in monogerm (2MOT) and multigerm (2MUF) hybrid parents in successive years. PO_14 to PO_20 represent hybrids, which were produced by combining the respective monogerm and multigerm parent between in six successive generations. The x-axis graphically represents the remaining genetic fragment from the ALS inhibitor herbicide tolerant donor SU-12-1 on chromosome 5 linked to the favorable BvALS_W569L allele at the center of the scale and labelled ALS which confers ALS inhibitor tolerance. Genetic distances are calculated based on sugar beet genetic map ZRINT1601. Upstream and downstream directions refer to the 5′ end and the 3′ end, respectively, of the coding strand of the BvALS gene. M1 to M13 and Mals refer to the position of the markers of Table 1.

FIG. 2: Box plots of sugar yield performance of experimental hybrids formed by recombinants of the 2MUF and 2MOT pools as graphically represented in FIG. 1. Box plots summarize sugar yield data of all experimental hybrids produced for that respective year and yield data are given in relation to a standard group and are adjusted over years. P014 to P019 hybrids correspond to those hybrids comprising the genomic fragments given in FIG. 1. P013 hybrids contain very large fragments of ALS inhibitor herbicide tolerant donor SU-12-1 and were created before any screening of homologous recombination.

### DETAILED DESCRIPTION OF THE EMBODIMENTS

The inventors have unexpectedly discovered that reduction of the size of chromosomal fragments of chromosome 5 linked to BvALS_W569L introgressed into parent plants of different heterotic pools of Beta vulgaris from the same donor line comprising BvALS_W569L, led to the hybrid Beta vulgaris plants (obtained by selecting a parent plant from each heterotic parent pool homozygous, crossing, harvesting the progeny seed and growing the hybrid plants), homozygous for the BvALS_W569L allele, with increasing sugar yield or biomass yield, paralleling the decreasing size of chromosome 5 fragments obtained from the donor linked to BvALS_W569L.

Without intending to be bound by any particular mode of action, it is thought that the reduction of the size of introgressed donor chromosomal fragments reduces the presence of homozygous alleles in hybrid Beta vulgaris plants homozygous for the BvALS_W569 allele and/or decreases the likelihood of unfavorable alleles present in the introgressed chromosome 5 fragments from the donor, thereby increasing heterosis and decreasing linkage drag.

In a first aspect, the invention provides a Beta vulgaris hybrid plant, or hybrid seeds, or a part thereof, comprising an ALS-herbicide tolerant endogenous ALS gene allele which is homozygously present on chromosome 5 in said Beta vulgaris hybrid plant, whereby the ALS-herbicide tolerant endogenous ALS gene allele preferably encodes an ALS protein comprising leucine at position 569, and whereby the Beta vulgaris hybrid plant can be obtained by crossing two parent Beta vulgaris plants, preferably from different heterotic pools, wherein each parent plant comprises the ALS-herbicide tolerant endogenous ALS gene allele in homozygous state and wherein the ALS-herbicide tolerant endogenous ALS gene allele in each parent plant is introgressed from the same ALS-herbicide tolerant endogenous ALS gene allele donor plant, characterized in that chromosomal region of chromosome 5 introgressed from said ALS-herbicide tolerant endogenous ALS gene allele donor plant and located upstream and/or downstream of the ALS inhibitor tolerant endogenous ALS gene allele in said parent plant is sufficiently small to avoid or decrease inbred depression and/or increase heterosis and/or increase sugar yield or biomass in said Beta vulgaris hybrid plant.

As used herein, “homozygous” or “homozygously” indicates that a plant has a copy of the same allele at the same locus on each of the corresponding chromosomes of the diploid pair of chromosomes, including a copy of the BvALS_W569L allele at the ALS gene locus. Homozygous may also be used to indicate the presence of the same introgressed chromosome 5 fragment from the donor plant, or a part of such fragment. As used herein, “heterozygous” or “heterozygously” indicates that a plant has a copy of the different allele at the same locus on each of the corresponding chromosomes of the diploid pair of chromosomes.

As used herein, a BvALS_W569L allele is a mutant allele of an endogenous Beta vulgaris ALS gene, encoding an ALS protein wherein the amino acid at position 569 is leucine instead of the normally occurring tryptophan. Such a mutant allele endows Beta vulgaris plants comprising it with tolerance to various ALS inhibitor herbicides, as hereinafter described in more detail. An ALS protein wherein the amino acid at position 569 is leucine instead of the normally occurring tryptophan is set forth in SEQ ID NO 16. The ALS protein wherein the amino acid at position 569 is leucine instead of the normally occurring tryptophan may however vary at other amino acid positions than position 569 and may have an amino acid sequence which is has at least 90, 95, 97, 98, or 99% sequence identity or is 100% identical to the polypeptide or protein encoded by BvALS_W697L as set forth in SEQ ID NO 16 provided it contains a leucine at position 569 of the amino acid sequence.

The BvALS_W569L allele may comprise the nucleotide sequence of SEQ ID NO: 15 wherein a transversion of the “G” nucleotide at a position corresponding to position 1706 to a “T” nucleotide occurred compared to the wild type allele. The ALS allele may also vary at other nucleotide position than position 1706 and may have a nucleotide sequence which is at least 90, 95, 97, 98, or 99% sequence identity or is 100% identical to the nucleotide sequence of BvALS_W697L as set forth in SEQ ID NO 15 provided it contains a TTG codon at position 1705-1707 of SEQ ID NO 15.

Beta vulgaris plants comprising the BvALS_W569L allele are less sensitive to an ALS inhibitor, more preferably it is at least 100 times less sensitive, more preferably, 500 times, even more preferably 1000 times and most preferably less than 2000 times than Beta vulgaris plants comprising the wild-type allele. Less sensitive when used herein may, vice versa, be seen as “more tolerable” or “more resistant”. Similarly, “more tolerable” or “more resistant” may, vice versa, be seen as “less sensitive”. For example, B. vulgaris plants comprising the BvALS_W569L allele are at least 2000 times less sensitive to the ALS inhibitor herbicide foramsulfuron (a member of the ALS inhibitor subclass “sulfonylurea herbicides”) compared to B. vulgaris plants comprising a BvALS wild type allele.

As used herein a BvALS-WT or “wild-type allele,” “wild-type ALS allele”, “wild-type ALS gene” or “wild-type ALS polynucleotide” refer to a nucleotide sequence that encodes an ALS protein that lacks the W569L substitution. Reference nucleotide sequences and amino acid sequences corresponding to such BvALS_WT or encoded protein are set forth in SEQ ID NO 17 and SEQ ID NO: 18 respectively.

Preferably, the BvALS_W569L comprise only the substitution at position 569 of the encoded ALS protein as sole mutation. Reference B. vulgaris seeds comprising the BvALS_W569L have been deposited as NCIMB 41705. Reference seed deposited as NCIMB 43836, NCIMB 43837 or NCIMB 43838 on Aug. 4, 2021 also contain the BvALS_W569L allele.

The presence of the BvALS_W569L in progeny plants may be followed by phenotype, i.e. tolerance to ALS inhibitor herbicides, or may be determined using KASP marker Mals (SEQ ID NO: 14) by the determining the presence of the G-nucleotide at the variant position of the marker.

The size of the chromosomal fragments of chromosome 5 from the donor ALS inhibitor tolerant Beta vulgaris line which is introgressed into the (elite) inbred parent lines of the different heterotic pools can be estimated by using polymorphic markers which differ in at least one nucleotide position between the donor line and the (elite) parent lines and which are located around i.e. upstream or downstream the BvALS allele. Such polymorphic markers exhibit in one form, or one allele, the nucleotide present in the nucleotide sequence of the donor line and in another form, or another allele, the nucleotide present in the nucleotide sequence of the (elite) parent line. Finding in one plant upon analysis the simultaneous presence of the BvALS_W569L allele and the presence of the marker allele from the (elite) parent line for one or more of the polymorphic markers located around the BvALS_W569L allele indicates that a recombination event occurred in the chromosome region between the polymorphic marker and the BvALS_W569L allele. Accordingly, the end of the introgressed chromosome 5 fragment from the donor or source line is delineated or flanked by that marker, at the maximum. The size of the introgressed fragment can be smaller. Preferably, the polymorphic markers located more distal (with reference to the BvALS_W569L allele) than the marker for which the marker allele of the (elite) line was determined also all exhibit the marker allele of the (elite) lines. By determining the presence of marker alleles indicative of the (elite) parent lines for polymorphic markers located upstream and downstream of the BvALS_W569L allele, the size of the introgressed chromosomal fragment (interval) from the source line can be determined. Such markers are said to flank the introgressed chromosomal fragment (interval) from the donor line.

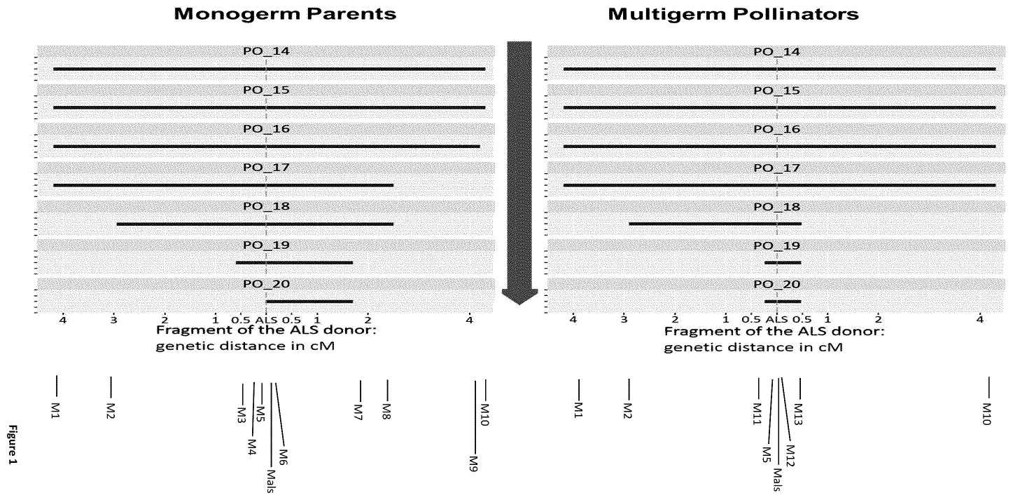

Examples of such markers are listed in Table 1. Markers useful for determining the maximum size of the introgressed chromosome 5 fragment from the donor line along with the BvALS_W569L allele comprise the indicated nucleotide sequence (but may comprise additional nucleotides at the 5′ and 3′ end). The polymorphic nucleotide is indicated using standard symbols encompassing possible nucleotide variations. Also indicated are the nucleotide/allele for the polymorphic marker as present in the donor SU-12-1 line and as present in the (elite) parent lines of the heterotic pools. The location of the markers relative to the BvALS_W569L allele are indicated in Tables 2 and 3 and schematically represented in FIG. 1.

Accordingly, in a further aspect of the invention, hybrid B. vulgaris plants and seeds thereof are provided as described above, wherein one or both of the parent plants of such hybrids comprise a chromosomal region of chromosome 5 introgressed from the ALS-herbicide tolerant endogenous ALS gene allele donor plant and comprising the ALS-herbicide tolerant endogenous ALS gene allele, such as the BvALS_W569L allele on a chromosomal interval, which is flanked on one side of the ALS-herbicide tolerant endogenous ALS gene allele by a marker selected from the group consisting of marker M1 (SEQ ID NO. 1), marker M2 (SEQ ID NO. 2), marker M3 (SEQ ID NO. 3), marker M4 (SEQ ID NO. 4), marker M11 ((SEQ ID NO. 11), marker M14 (SEQ ID NO. 19), marker M15 (SEQ ID NO: 20) and marker M5 (SEQ ID NO. 1) and on the other side of the ALS-herbicide tolerant endogenous ALS gene allele is flanked by a marker selected from the group consisting of marker M6 (SEQ ID NO. 6), marker M7 (SEQ ID NO. 7), marker M8 (SEQ ID NO. 8), marker M9 (SEQ ID NO. 9), marker M12 (SEQ ID NO. 12), marker M13 (SEQ ID NO. 13), marker M10 (SEQ ID NO. 10), marker M16 (SEQ ID NO. 21) and marker M17 (SEQ ID NO. 22).

Thus, the hybrid B. vulgaris plants or seeds or plants thereof may comprise on their chromosomes 5 an introgressed chromosomal region comprising a BvALS_W569L allele flanked by markers M1 and M6; markers M1 and M7; markers M1 and M8; markers M1 and M9; markers M1 and M10; markers M1 and M16; markers M1 and M17; markers M2 and M6; markers M2 and M7; markers M2 and M8; markers M2 and M9; markers M2 and M10; markers M2 and M16; markers M2 and M17; markers M3 and M6; markers M3 and M7; markers M3 and M8; markers M3 and M9; markers M3 and M10; markers M3 and M16; markers M3 and M17; markers M4 and M6; markers M4 and M7; markers M4 and M8; markers M4 and M9; markers M4 and M10; markers M4 and M16; markers M4 and M17; markers M5 and M6; markers M5 and M7; markers M5 and M8; markers M5 and M9; markers M5 and M10; markers M5 and M16; markers M5 and M17; markers M14 and M6; markers M14 and M7; markers M14 and M8; markers M14 and M9; markers M14 and M10; markers M14 and M16; markers M14 and M17; markers M15 and M6; markers M15 and M7; markers M15 and M8; markers M15 and M9; markers M15 and M10; markers M15 and M16; markers M15 and M17; markers M1 and M12; markers M1 and M13; markers M1 and M10; markers M2 and M12; markers M2 and M13; markers M2 and M10; markers M5 and M12; markers M5 and M13; markers M5 and M10; markers M11 and M12; markers M11 and M13; markers M11 and M10; markers M11 and M16; markers M11 and M17; markers M14 and M12; markers M14 and M13; markers M15 and M12; or markers M15 and M13. The introgressed chromosomal fragment on both chromosomes 5 may be twice identical or may be a different introgressed chromosomal fragment each selected from the above list. Particularly useful hybrid B. vulgaris plants comprise a chromosomal region introgressed from the ALS-herbicide tolerant endogenous ALS gene allele donor plant of chromosome 5 and comprising said ALS-herbicide tolerant endogenous ALS gene allele which is flanked by marker M5 and marker M6 on one of the chromosomes 5 and by marker M5 and M12 on the other chromosome 5. Further particularly useful hybrid B. vulgaris plants comprise the chromosomal region introgressed from said ALS-herbicide tolerant endogenous ALS gene allele donor plant of chromosome 5 and comprising said ALS-herbicide tolerant endogenous ALS gene allele, as present in one or more seeds deposited with the NCIMB, Aberdeen, UK, under Number NCIMB 43836, NCIMB 43837 or NCIMB 43838 on Aug. 4, 2021, or are grown out of or derived from a seed as deposited with the NCIMB, Aberdeen, UK, under Number NCIMB 43836, NCIMB 43837 or NCIMB 43838 on Aug. 4, 2021.

The hybrid Beta vulgaris plants, seeds or parts described herein have a yield potential that is inversely correlated with the size of the introgressed chromosome 5 fragments comprising BvALS_W569L from the source line. Hybrid Beta vulgaris plants has herein described may have a sugar yield of at least 80%, at least 85%, at least 90%, at least 91%, at least 92%, at least 93%, at least 94%, at least 95%, at least 96%, at least 97%, at least 98%, at least 99%, or equal to or greater to the sugar yield of a control Beta vulgaris hybrid plant or isogenic Beta vulgaris hybrid plant or check (elite) B. vulgaris line, i.e. a B. vulgaris (elite) line, preferably obtained from parent plants from different heterotic groups and which comprising a wild-type allele of the ALS encoding gene on chromosome 5 in homozygous state, and thus also do not comprise an introgressed chromosome 5 fragment from the ALS inhibitor tolerant donor line.

The hybrid Beta vulgaris plants, seeds or parts described herein carry a chromosomal region of chromosome 5 introgressed from said ALS-herbicide tolerant endogenous ALS gene allele donor plant and located upstream and/or downstream of the ALS inhibitor tolerant endogenous ALS gene allele in said parent plant which is sufficiently small to avoid or decrease inbred depression and/or increase heterosis and/or increase sugar yield or biomass yield in said Beta vulgaris hybrid plant. “Sufficiently small” means that the chromosomal region is freed from donor material upstream and/or downstream of the ALS inhibitor tolerant endogenous ALS gene allele in said parent plant, preferably by truncation of the region, whereby the region remains a continuous region derived from the donor. In this way a decrease of inbred depression and/or an increase of heterosis and/or an increase sugar yield or biomass yield in said Beta vulgaris hybrid plant is detectable/measurable. Preferably, the decrease of inbred depression and/or the increase of heterosis and/or the increase sugar yield or biomass yield is detectable/measurable in comparison with a control Beta vulgaris hybrid plant or isogenic Beta vulgaris hybrid plant or check (elite) B. vulgaris line, i.e. a B. vulgaris (elite) line, preferably obtained from parent plants from different heterotic groups and which comprising a wild-type allele of the ALS encoding gene on chromosome 5 in homozygous state, and thus also do not comprise an introgressed chromosome 5 fragment from the ALS inhibitor tolerant donor line. The term “non-transgenic” or “non-transgene” or “non-genetically modified” means that no introduction of the respective gene has occurred via an appropriate biological carrier or by any other physical means. However, a mutated gene can be transferred through pollination, either naturally or via a breeding process to produce another non-transgenic plant.

The term “chromosomal fragment”, “chromosomal region” or “chromosomal interval” means a continuous linear segment on a genomic DNA which is present in an individual chromosome in a plant or on a chromosome fragment and which is usually defined through two markers which may represent the end points of the interval on the distal and proximal side. In this regard, the markers may themselves also be a part of the interval. Furthermore, two different intervals might overlap. In the description, an interval is specified by the statement “flanked by marker A and marker B”.

The term “introgression” as used herein means the transfer of at least one desired gene allele on a genetic locus of a genetic background into another. As an example, an introgression of a desired gene allele at a specific locus may be transferred to a descendant by sexual crossing between two parents of the same species. Alternatively, for example, the transfer of a gene allele may also occur by recombination between two donor genomes in a fused protoplast, wherein at least one donor protoplast carries the desired gene allele in its genome. In each case the descendants, which then comprise the desired gene allele, can then be backcrossed again with a line which comprises a preferred genetic background and can be selected for the desired gene allele. The result is fixing of the desired gene allele in a selected genetic background.

Parts of plants may be attached to or separate from a whole intact plant. Such parts of a plant include, but are not limited to, organs, tissues, and cells of a plant, and preferably seeds.

In another aspect of the invention, DNA molecules are provided consisting of the chromosomal region of chromosome 5 of a Beta vulgaris ALS inhibitor herbicide tolerant plant comprising an ALS-herbicide tolerant endogenous ALS gene, reference seed of which has been deposited as NCIMB 41705, which is located (only or exclusively) on a chromosomal interval flanked on one side of the ALS-herbicide tolerant endogenous ALS gene allele by a marker selected from the group consisting of marker M1, marker M2, marker M3, marker M4, marker M11, marker M14, marker M15 and marker M5, and on the other side of the ALS-herbicide tolerant endogenous ALS gene allele is flanked by a marker selected from the group consisting of marker M6, marker M7, marker M8, marker M9 and marker M10, marker M12, marker M13, marker M16 and marker M17. Particularly useful DNA molecules are those wherein the chromosomal region is flanked by markers M5 and M6 or wherein the chromosomal region is flanked by markers M4 and M7, or wherein the chromosomal region is flanked by markers M5 and M12, or wherein the chromosomal region is flanked by M11 and M13, or wherein the chromosomal region is flanked by M5 and M16, or wherein the chromosomal region is flanked by M14 and M17.

Examples of such DNA molecules are listed in Tables 2, 3 and 4, with an indication of the size of the introgressed fragment of chromosome 5 derived from the source ALS inhibitor tolerant line. This size is expressed in centimorgan (cM), a unit to express genetic distance. A genomic sequence build of the complete sequence for sugar beets is publicly available and can be found at EnsemblPlants website https://plants.ensembl.org/Beta_vulgaris/Info/Index as RefBeet 1.2.2 Accession GCA_000511025 (https://www.ebi.ac.uk/ena/browser/view/GCA_000511025.2).

A person skilled in the art can thus easily locate the marker nucleotide sequences herein provided on the complete genome sequence of sugar beets, by sequence comparison using computer programs and algorithms. For example, BLAST, which stands for Basic Local Alignment Search Tool (Altschul, Nucl. Acids Res. 25 (1997), 3389-3402; Altschul, J. Mol. Evol. 36 (1993), 290-300; Altschul, J. Mol. Biol. 215 (1990), 403-410), can be used to search for local sequence alignments.

Once the nucleotide sequence of the markers herein described have been physically allocated to the corresponding position in the genomic nucleotide sequence map of the chromosome 5 contigs, the person skilled in the art can identify the physical size of the introgressed chromosome 5 fragments flanked by such markers in kilobases, and can deduce the consensus nucleotide sequence of the fragment flanked by such markers.

Also described are Beta vulgaris plants, particularly (elite) Beta vulgaris plants, such as sugar beet plants or fodder beet plants, which comprise the DNA molecules as herein described, in homozygous or heterozygous state, and which can be used as parent plant to obtain the hybrid Beta vulgaris plants or seeds described herein. To this end, such plants are cross-pollinated and the progeny seeds are harvested. One of the parent plants may be male-sterile (female plant) and be pollinated with pollen from the male parent plant. Methods to obtain male-sterile Beta vulgaris plants are well known in the art.

The B. vulgaris plants of the present invention and harvestable parts thereof are agronomically exploitable. “Agronomically exploitable” means that the B. vulgaris plants and parts thereof are useful for agronomical purposes. For example, the B. vulgaris plants should serve for the purpose of being useful for sugar production, bio fuel production (such as biogas, biobutanol), ethanol production, betaine and/or uridine production and use of a hybrid Beta vulgaris plant as herein described for the production of sugar, ethanol, betaine and/or uridine is also envisioned. The B. vulgaris plants or parts thereof could be used as animal feed stuff or to produce animal feed.

A “plant of the species Beta vulgaris” or a “Beta vulgaris plant” comprises a plant of the subspecies Beta vulgaris subsp. vulgaris such as Beta vulgaris subsp. vulgaris var. altissima (sugar beet in a narrower sense), Beta vulgaris ssp. vulgaris var. vulgaris (chard), Beta vulgaris ssp. vulgaris var. conditiva (beetroot/red beet), Beta vulgaris ssp. vulgaris var. crassa/alba (fodder beet).

An example for an agronomically exploitable B. vulgaris plant is sugar beet. A sugar beet plant of the present invention when cultivated in an area of one hectare yields (about 80,000 to 90,000 sugar beets) should preferably serve for the production of at least 4 tons of sugar.

A sugar beet plant of the present invention should preferably contain a sugar content between 15-20%, preferably at least 17% so as to be agronomically exploitable. Thus, sugar beet plants that contain a sugar content between 15-20%, preferably at least 17% are a preferred embodiment of the present invention.

Yet another example of an agronomically exploitable B. vulgaris plant is fodder beet. Fodder beets may be used either to produce animal feed by chopping the harvested beets and feeding to animals like cattle and cows purely or mixed with other feeding components, or may be used as grazing fodder beets.

Another aspect of the present invention is the use of the Beta vulgaris plants described herein and/or the harvestable parts or propagation material described herein for the manufacture/breeding of further Beta vulgaris plants.

Also envisioned are methods for identifying a genomic fragment of chromosome 5 in (elite) Beta vulgaris plants, introgressed from an ALS-herbicide tolerant Beta vulgaris donor plant, wherein the genomic fragment comprises an ALS-herbicide tolerant endogenous ALS gene allele, or for identifying/selecting a Beta vulgaris plant comprising said genomic fragment of chromosome 5, the methods comprising the steps of:
(a) identifying the presence of the ALS-herbicide tolerant endogenous ALS gene allele in B. vulgaris plant such as an (elite) B. vulgaris plant by a phenotypic or a marker-assisted method; and
(b) identifying the presence of the allele/nucleotide as present in the (elite) Beta vulgaris plant at nucleotide position 31 of one or more markers selected from the group of marker M1 (SEQ ID NO. 1), marker M2 (SEQ ID NO. 2), marker M3 (SEQ ID NO. 3), marker M4 (SEQ ID NO. 4), marker M5 (SEQ ID NO. 5), marker M6 (SEQ ID NO. 6), marker M7 (SEQ ID NO. 7), marker M8 (SEQ ID NO. 8), marker M9 (SEQ ID NO. 9), marker M10 (SEQ ID NO. 10), marker M11 (SEQ ID NO. 11) and marker M13 (SEQ ID NO. 13), at position 30 of marker M12 (SEQ ID NO. 12) or at position 101 of one or more markers selected from the group of marker 14 (SEQ ID NO. 19), marker 15 (SEQ ID NO. 20), marker 16 (SEQ ID NO. 21) and marker M17 (SEQ ID NO. 22);
(c) optionally selecting an ALS inhibitor tolerant Beta vulgaris plant, wherein said plant exhibits a decreased inbred depression and/or an increased heterosis and/or an increased sugar yield or biomass yield.

Another method for identifying a genomic fragment of chromosome 5 in a (elite) Beta vulgaris plant, introgressed from an ALS-herbicide tolerant Beta vulgaris donor plant, said genomic fragment comprising an ALS-herbicide tolerant endogenous ALS gene allele, or for identifying/selecting a Beta vulgaris plant comprising said genomic fragment of chromosome 5, comprises the steps of
(a) identifying the presence of the ALS-herbicide tolerant endogenous ALS gene allele in said plant by a phenotypic or a marker-assisted method; and
(b) identifying the presence of at least one allele/nucleotide on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22);
(c) and optionally selecting an ALS inhibitor tolerant Beta vulgaris plant, wherein said plant exhibits a decreased inbred depression and/or an increased heterosis and/or an increased sugar yield or biomass yield.

A further method for identifying a genomic fragment of chromosome 5 in (elite) Beta vulgaris plants, introgressed from an ALS-herbicide tolerant Beta vulgaris donor plant, said genomic fragment comprising an ALS-herbicide tolerant endogenous ALS gene allele, comprises the steps of
(a) identifying the presence of at least one first allele/nucleotide on a chromosomal interval flanked by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and by the ALS-herbicide tolerant endogenous ALS gene allele, and
(b) identifying the presence of at least one second allele/nucleotide on a chromosomal interval flanked by the ALS-herbicide tolerant endogenous ALS gene allele and by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22)
(c) and optionally selecting an ALS inhibitor tolerant Beta vulgaris plant, wherein said plant exhibits a decreased inbred depression and/or an increased heterosis and/or an increased sugar yield or biomass yield.

The alleles or nucleotides as present in the (elite) Beta vulgaris plant are indicated in Table 1. Thus, steps of the identifying method may comprise determining the presence of a G at nucleotide position 31 of marker M1 (SEQ ID NO. 1); the presence of a T at nucleotide position 31 of marker M2 (SEQ ID NO. 2); the presence of a G at nucleotide position 31 of marker M3 (SEQ ID NO. 3); the presence of an A at nucleotide position 31 of marker M4 (SEQ ID NO. 4); the presence of a C at nucleotide position 31 of marker M5 (SEQ ID NO. 5); the presence of a C at nucleotide position 31 of marker M6 (SEQ ID NO. 6); the presence of a C at nucleotide position 31 of marker M7 (SEQ ID NO. 7); the presence of a C at nucleotide position 31 of marker M8 (SEQ ID NO. 8); the presence of an A at nucleotide position 31 of marker M9 (SEQ ID NO. 9); the presence of an A at nucleotide position 31 of marker M10 (SEQ ID NO. 10); the presence of an A at nucleotide position 31 of marker M11 (SEQ ID NO. 11); the presence of a T at nucleotide position 30 of marker M12 (SEQ ID NO. 12); the presence of an A at nucleotide position 31 of marker M13 (SEQ ID NO. 13); the presence of an C at nucleotide position 101 of marker M14 (SEQ ID NO. 19); the presence of an T at nucleotide position 101 of marker M15 (SEQ ID NO. 20); the presence of an G at nucleotide position 101 of marker M16 (SEQ ID NO. 21); or the presence of an C at nucleotide position 101 of marker M17 (SEQ ID NO. 22).

The presence of the BvALS_W569L allele can be determined by identifying the presence of a G nucleotide at nucleotide position 31 of marker Mals (SEQ ID NO. 14).

In yet another aspect of the invention, markers are provided for determining the presence and size of introgressed chromosome 5 fragments from the donor line. These markers comprise DNA molecule comprising the sequence of any one of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 19, SEQ ID NO. 20, SEQ ID NO. 21 or SEQ ID NO. 22.

Markers can be used to identify plants of the present invention using any genotypic analysis method. Genotypic evaluation of the plants includes using techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Allele-specific PCR (AS-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as “Microsatellites”. Additional compositions and methods for analyzing the genotype of the plants provided herein include those methods disclosed in U.S. Publication No. 2004/0171027, U.S. Publication No. 2005/02080506, and U.S. Publication No. 2005/0283858.

A particular useful assay method for genotyping of single nucleotide polymorphism markers is the KASP assay (Competitive allele specific PCR) as described e.g. by Chunlin He, John Holme and Jeffrey Anthony in “SNP genotyping: the KASP assay” Methods Mol Biol 2014; 1145:75-86 doi: 10.1007/978-1-4939-0446-4_7.

In another aspect of the invention, use of one or more one or more ALS inhibitor herbicide(s) for controlling unwanted vegetation in Beta vulgaris growing areas wherein the Beta vulgaris plants are hybrid Beta vulgaris plants as herein described, is provided.

The ALS inhibitor herbicides may belong to any one of those listed in the above numbered embodiments of the invention The “CAS RN” stated in square brackets behind the names (common names) mentioned under groups A to C corresponds to the “chemical abstract service registry number”, a customary reference number which allows the substances named to be classified unambiguously, since the “CAS RN” distinguishes, inter alia, between isomers including stereoisomers. The listed compounds moreover are further indicated by a number between brackets, such as A1-1 etc., used further hereinafter.

In the context of the invention, “tolerance” or “tolerant” means that the application of one or more ALS inhibitor herbicide(s) belonging to any of the above defined groups (A), (B), (C) does not show any apparent effect(s) concerning the physiological functions/phytotoxicity when applied to the hybrid Beta vulgaris plant, especially sugar beet, as herein described and wherein the application of the same amount of the respective ALS inhibitor herbicide(s) on non-tolerant Beta vulgaris plants leads to significant negative effects concerning plant growth, its physiological functions or shows phytotoxic symptoms. Quality and quantity of the observed effects may depend on the chemical composition of the respective ALS inhibitor herbicide(s) applied, dose rate and timing of the application as well growth conditions/stage of the treated plants.

A useful ALS inhibitor herbicide comprises foramsulfuron [CAS RN 173159-57-4](=A1-13) and thiencarbazone-methyl [CAS RN 317815-83-1](=A2-3).

Another ALS inhibitor herbicide which may be used for control of unwanted vegetation in Beta vulgaris (preferably sugar beet) growing areas in which the Beta vulgaris (preferably sugar beet) plants are hybrid B. vulgaris plants as herein described is imazamox [CAS RN 114311-32-9](=B1-2).

Another ALS inhibitor herbicide which may be used for control of unwanted vegetation in Beta vulgaris (preferably sugar beet or fodder beet) growing areas in which the Beta vulgaris (preferably sugar beet or fodder beet plants) plants are hybrid B. vulgaris plants as herein described is bispyribac-sodium [CAS RN 125401-92-5](=C1-1).

Another ALS inhibitor herbicide which may be used for control of unwanted vegetation in Beta vulgaris (preferably sugar beet) growing areas in which the Beta vulgaris (preferably sugar beet or fodder beet plants) plants are hybrid B. vulgaris plants as herein described is triflusulfuron-methyl.

Additionally, the ALS inhibitor herbicide(s) to be used on hybrid B. vulgaris plants as herein described may comprise further components, for example agrochemically active compounds of a different type of mode of action and/or the formulation auxiliaries and/or additives customary in crop protection, or may be used together with these.

In a preferred embodiment, the herbicide combinations to be used according to the invention comprise effective amounts of the ALS inhibitor herbicide(s) belonging to groups (A), (B) and/or (C) and/or have synergistic actions. The synergistic actions can be observed, for example, when applying one or more ALS inhibitor herbicide(s) belonging to groups (A), (B), and/or (C) together, for example as a coformulation or as a tank mix; however, they can also be observed when the active compounds are applied at different times (splitting). It is also possible to apply the herbicides or the herbicide combinations in a plurality of portions (sequential application), for example pre-emergence applications followed by post-emergence applications or early postemergence applications followed by medium or late post-emergence applications.

Preference is given here to the joint or almost simultaneous application of the ALS inhibitor herbicides belonging to groups (A), (B) and/or (C) of the combination in question.

The synergistic effects permit a reduction of the application rates of the individual ALS inhibitor herbicides, a higher efficacy at the same application rate, the control of species which were as yet uncontrolled (gaps), control of species which are tolerant or resistant to individual ALS inhibitor herbicides or to a number of ALS inhibitor herbicides, an extension of the period of application and/or a reduction in the number of individual applications required and—as a result for the user—weed control systems which are more advantageous economically and ecologically.

The herbicides to be used according to this invention are all acetolactate synthase (ALS) inhibitor herbicides (which might alternatively and interchangeably also be named as “ALS inhibiting herbicides”) and thus inhibit protein biosynthesis in plants.

The application rate of the ALS inhibitor herbicides belonging to groups (A), (B) or (C) (as defined above) can vary within a wide range, for example between 0.001 g and 1500 g of ai/ha (ai/ha means here and below “active substance per hectare”=based on 100% pure active compound). Applied at application rates of from 0.001 g 20 to 1500 g of ai/ha, the herbicides belonging to classes A, Band C according to this invention, preferably the compounds A1-1; A1-4; A1-8; A1-9; A1-12; A1-13; A1-16; A1-17; A1-18; A1-19; A1-20; A1-28; A1-29; A1-31; A1-39; A1-41; A1-83; A1-87; A2-2; A2-3; A3-3; A3-5; A3-7, A4-3, control, when used by the pre- and post-emergence method, a relatively wide spectrum of harmful plants, for example of annual and perennial mono- or dicotyledonous weeds, and also of unwanted crop plants (together also defined as “unwanted vegetation).

In many applications according to the invention, the application rates are generally lower, for example in the range of from 0.001 g to 1000 g of ai/ha, preferably from 30 0.1 g to 500 g of ai/ha, particularly preferably from 0.5 g to 250 g of ai/ha, and even more preferably 1.0 g to 200 g of ai/ha. In cases where the application of several ALS inhibitor herbicides is conducted, the quantity represents the total quantity of all of the applied ALS inhibitor herbicides. For example, the combinations according to the invention of ALS inhibitor herbicides (belonging to groups (A), (B) and/or (C)) allow the activity to be enhanced synergistically in a manner which, by far and in an unexpected manner, exceeds the activities which can be achieved using the individual ALS inhibitor herbicides (belonging to groups (A), (B) and/or (C)).

For combinations of ALS inhibitor herbicides, the preferred conditions are illustrated below.

Of particular interest according to present invention is the use of herbicidal compositions for control of unwanted vegetation in Beta vulgaris plants, preferably in sugar beet plants having a content of the following ALS inhibitor herbicides:

$$( A 1 - 1 ) + ( A 1 - 4 ) ; ( A 1 - 1 ) + ( A 1 - 8 ) ; A 1 - 1 ) + ( A 1 - 9 ) ;$$

$$( A 1 - 1 ) + ( A 1 - 12 ) ; ( A 1 - 1 ) + ( A 1 - 13 ) ; ( A 1 - 1 ) + ( A 1 - 16 ) ;$$

$$( A 1 - 1 ) + ( A 1 - 17 ) ; ( A 1 - 1 ) + ( A 1 - 18 ) ; ( A 1 - 1 ) + ( A 1 - 19 ) ;$$

$$( A 1 - 1 ) + ( A 1 - 20 ) ; ( A 1 - 1 ) + ( A 1 - 28 ) ; ( A 1 - 1 ) + ( A 1 - 29 ) ;$$

$$( A 1 - 1 ) + ( A 1 - 31 ) ; ( A 1 - 1 ) + ( A 1 - 39 ) ; ( A 1 - 1 ) + ( A 1 - 41 ) ;$$

$$( A 1 - 1 ) + ( A 1 - 83 ) ; ( A 1 - 1 ) + ( A 1 - 87 ) ; ( A 1 - 1 ) + ( A 2 - 2 ) ;$$

$$( A 1 - 1 ) + ( A 2 - 3 ) ; ( A 1 - 1 ) + ( A 3 - 3 ) ; ( A 1 - 1 ) + ( A 3 - 5 ) ;$$

$$( A 1 - 1 ) + ( A 3 - 7 ) ; ( A 1 - 1 ) + ( A 4 - 1 ) ; ( A 1 - 1 ) + ( A 4 - 2 ) ;$$

$$( A 1 - 1 ) + ( A 4 - 3 ) ; ( A 1 - 4 ) + ( A 1 - 8 ) ; ( A 1 - 4 ) + ( A 1 - 9 ) ;$$

$$( A 1 - 4 ) + ( A 1 - 12 ) ; ( A 1 - 4 ) + ( A 1 - 13 ) ; ( A 1 - 4 ) + ( A 1 - 16 ) ;$$

$$( A 1 - 4 ) + ( A 1 - 17 ) ; ( A 1 - 4 ) + ( A 1 - 18 ) ; ( A 1 - 4 ) + ( A 1 - 19 ) ;$$

$$( A 1 - 4 ) + ( A 1 - 20 ) ; ( A 1 - 4 ) + ( A 1 - 28 ) ; ( A 1 - 4 ) + ( A 1 - 29 ) ;$$

$$( A 1 - 4 ) + ( A 1 - 31 ) ; ( A 1 - 4 ) + ( A 1 - 39 ) ; ( A 1 - 4 ) + ( A 1 - 41 ) ;$$

$$( A 1 - 4 ) + ( A 1 - 83 ) ; ( A 1 - 4 ) + ( A 1 - 87 ) ; ( A 1 - 4 ) + ( A 2 - 2 ) ;$$

$$( A 1 - 4 ) + ( A 2 - 3 ) ; ( A 1 - 4 ) + ( A 3 - 3 ) ; ( A 1 - 4 ) + ( A 3 - 5 ) ;$$

$$( A 1 - 4 ) + ( A 3 - 7 ) ; ( A 1 - 4 ) + ( A 4 - 1 ) ; ( A 1 - 4 ) + ( A 4 - 2 ) ;$$

$$( A 1 - 4 ) + ( A 4 - 3 ) ; ( A 1 - 8 ) + ( A 1 - 9 ) ; ( A 1 - 8 ) + ( A 1 - 12 ) ;$$

$$( A 1 - 8 ) + ( A 1 - 13 ) ; ( A 1 - 8 ) + ( A 1 - 16 ) ; ( A 1 - 8 ) + ( A 1 - 17 ) ;$$

$$( A 1 - 8 ) + ( A 1 - 18 ) ; ( A 1 - 8 ) + ( A 1 - 19 ) ; ( A 1 - 8 ) + ( A 1 - 20 ) ;$$

$$( A 1 - 8 ) + ( A 1 - 28 ) ; ( A 1 - 8 ) + ( A 1 - 29 ) ; ( A 1 - 8 ) + ( A 1 - 31 ) ;$$

$$( A 1 - 8 ) + ( A 1 - 39 ) ; ( A 1 - 8 ) + ( A 1 - 41 ) ; ( A 1 - 8 ) + ( A 1 - 83 ) ;$$

$$( A 1 - 8 ) + ( A 1 - 87 ) ; ( A 1 - 8 ) + ( A 2 - 2 ) ; ( A 1 - 8 ) + ( A 2 - 3 ) ;$$

$$( A 1 - 8 ) + ( A 3 - 3 ) ; ( A 1 - 8 ) + ( A 3 - 5 ) ; ( A 1 - 8 ) + ( A 3 - 7 ) ;$$

$$A 1 - 8 ) + ( A 4 - 1 ) ; ( A 1 - 8 ) + ( A 4 - 2 ) ; ( A 1 - 8 ) + ( A 4 - 3 ) ;$$

$$( A 1 - 9 ) + ( A 1 - 12 ) ; ( A 1 - 9 ) + ( A 1 - 13 ) ; ( A 1 - 9 ) + ( A 1 - 16 ) ;$$

$$( A 1 - 9 ) + ( A 1 - 7 ) ; ( A 1 - 9 ) + ( A 1 - 18 ) ; ( A 1 - 9 ) + ( A 1 - 19 ) ;$$

$$( A 1 - 9 ) + ( A 1 - 20 ) ; ( A 1 - 9 ) + ( A 1 - 28 ) ; ( A 1 - 9 ) + ( A 1 - 29 ) ;$$

$$( A 1 - 9 ) + ( A 1 - 31 ) ; ( A 1 - 9 ) + ( A 1 - 39 ) ; ( A 1 - 9 ) + ( A 1 - 41 ) ;$$

$$( A 1 - 9 ) + ( A 1 - 83 ) ; ( A 1 - 9 ) + ( A 1 - 87 ) ; ( A 1 - 9 ) + ( A 2 - 2 ) ;$$

$$( A 1 - 9 ) + ( A 2 - 3 ) ; ( A 1 - 9 ) + ( A 3 - 3 ) ; ( A 1 - 9 ) + ( A 3 - 5 ) ;$$

$$( A 1 - 9 ) + ( A 3 - 7 ) ; ( A 1 - 9 ) + ( A 4 - 1 ) ; ( A 1 - 9 ) + ( A 4 - 2 ) ;$$

$$( A 1 - 9 ) + ( A 4 - 3 ) ; ( A 1 - 12 ) + ( A 1 - 13 ) ; ( A 1 - 12 ) + ( A 1 - 16 ) ;$$

$$( A 1 - 12 ) + ( A 1 - 17 ) ; ( A 1 - 12 ) + ( A 1 - 18 ) ; ( A 1 - 12 ) + ( A 1 - 19 ) ;$$

$$( A 1 - 12 ) + ( A 1 - 20 ) ; ( A 1 - 12 ) + ( A 1 - 28 ) ; ( A 1 - 12 ) + ( A 1 - 29 ) ;$$

$$( A 1 - 12 ) + ( A 1 - 31 ) ; ( A 1 - 12 ) + ( A 1 - 39 ) ; ( A 1 - 12 ) + ( A 1 - 41 ) ;$$

$$( A 1 - 12 ) + ( A 1 - 83 ) ; ( A 1 - 12 ) + ( A 1 - 87 ) ; ( A 1 - 12 ) + ( A 2 - 2 ) ;$$

$$( A 1 - 12 ) + ( A 2 - 3 ) ; ( A 1 - 12 ) + ( A 3 - 3 ) ; ( A 1 - 12 ) + ( A 3 - 5 ) ;$$

$$( A 1 - 12 ) + ( A 3 - 7 ) ; ( A 1 - 12 ) + ( A 4 - 1 ) ; ( A 1 - 12 ) + ( A 4 - 2 ) ;$$

$$( A 1 - 12 ) + ( A 4 - 3 ) ; ( A 1 - 13 ) + ( A 1 - 16 ) ; ( A 1 - 13 ) + ( A 1 - 17 ) ;$$

$$( A 1 - 13 ) + ( A 1 - 18 ) ; ( A 1 - 13 ) + ( A 1 - 19 ) ; ( A 1 - 13 ) + ( A 1 - 20 ) ;$$

$$( A 1 - 13 ) + ( A 1 - 28 ) ; ( A 1 - 13 ) + ( A 1 - 29 ) ; ( A 1 - 13 ) + ( A 1 - 31 ) ;$$

$$( A 1 - 13 ) + ( A 1 - 39 ) ; ( A 1 - 13 ) + ( A 1 - 41 ) ; ( A 1 - 13 ) + ( A 1 - 83 ) ;$$

$$( A 1 - 13 ) + ( A 1 - 87 ) ; ( A 1 - 13 ) + ( A 2 - 2 ) ; ( A 1 - 13 ) + ( A 2 - 3 ) ;$$

$$( A 1 - 13 ) + ( A 3 - 3 ) ; ( A 1 - 13 ) + ( A 3 - 5 ) ; ( A 1 - 13 ) + ( A 3 - 7 ) ;$$

$$( A 1 - 13 ) + ( A 4 - 1 ) ; ( A 1 - 13 ) + ( A 4 - 2 ) ; ( A 1 - 13 ) + ( A 4 - 3 ) ;$$

$$( A 1 - 16 ) + ( A 1 - 17 ) ; ( A 1 - 16 ) + ( A 1 - 18 ) ; ( A 1 - 16 ) + ( A 1 - 19 ) ;$$

$$( A 1 - 16 ) + ( A 1 - 20 ) ; ( A 1 - 16 ) + ( A 1 - 28 ) ; ( A 1 - 16 ) + ( A 1 - 29 ) ;$$

$$( A 1 - 16 ) + ( A 1 - 31 ) ; ( A 1 - 16 ) + ( A 1 - 39 ) ; ( A 1 - 16 ) + ( A 1 - 41 ) ;$$

$$( A 1 - 16 ) + ( A 1 - 83 ) ; ( A 1 - 16 ) + ( A 1 - 87 ) ; ( A 1 - 16 ) + ( A 2 - 2 ) ;$$

$$30 ( A 1 - 16 ) + ( A 2 - 3 ) ; ( A 1 - 16 ) + ( A 3 - 3 ) ; ( A 1 - 16 ) + ( A 3 - 5 ) ;$$

$$( A 1 - 16 ) + ( A 3 - 7 ) ; ( A 1 - 16 ) + ( A 4 - 1 ) ; ( A 1 - 16 ) + ( A 4 - 2 ) ;$$

$$( A 1 - 16 ) + ( A 4 - 3 ) ; A 1 - 17 ) + ( A 1 - 18 ) ; ( A 1 - 17 ) + ( A 1 - 19 ) ;$$

$$( A 1 - 17 ) + ( A 1 - 20 ) ; ( A 1 - 17 ) + ( A 1 - 28 ) ; ( A 1 - 17 ) + ( A 1 - 29 ) ;$$

$$( A 1 - 17 ) + ( A 1 - 31 ) ; ( A 1 - 17 ) + ( A 1 - 39 ) ; ( A 1 - 17 ) + ( A 1 - 41 ) ;$$

$$( A 1 - 17 ) + ( A 1 - 83 ) ; ( A 1 - 17 ) + ( A 1 - 87 ) ; ( A 1 - 17 ) + ( A 2 - 2 ) ;$$

$$( A 1 - 17 ) + ( A 2 - 3 ) ; ( A 1 - 17 ) + ( A 3 - 3 ) ; ( A 1 - 17 ) + ( A 3 - 5 ) ;$$

$$( A 1 - 17 ) + ( A 3 - 7 ) ; ( A 1 - 17 ) + ( A 4 - 1 ) ; ( A 1 - 17 ) + ( A 4 - 2 ) ;$$

$$( A 1 - 17 ) + ( A 4 - 3 ) ; ( A 1 - 18 ) + ( A 1 - 19 ) ; ( A 1 - 18 ) + ( A 1 - 20 ) ;$$

$$( A 1 - 18 ) + ( A 1 - 28 ) ; ( A 1 - 18 ) + ( A 1 - 29 ) ; ( A 1 - 18 ) + ( A 1 - 31 ) ;$$

$$( A 1 - 18 ) + ( A 1 - 39 ) ; ( A 1 - 18 ) + ( A 1 - 41 ) ; ( A 1 - 18 ) + ( A 1 - 83 ) ;$$

$$( A 1 - 18 ) + ( A 1 - 87 ) ; ( A 1 - 18 ) + ( A 2 - 2 ) ; ( A 1 - 18 ) + ( A 2 - 3 ) ;$$

$$( A 1 - 18 ) + ( A 3 - 3 ) ; ( A 1 - 18 ) + ( A 3 - 5 ) ( A 1 - 18 ) + ( A 3 - 7 ) ;$$

$$( A 1 - 18 ) + ( A 4 - 1 ) ; ( A 1 - 18 ) + ( A 4 - 2 ) ; ( A 1 - 18 ) + ( A 4 - 3 ) ;$$

$$( A 1 - 19 ) + ( A 1 - 20 ) ; ( A 1 - 19 ) + ( A 1 - 28 ) ; ( A 1 - 19 ) + ( A 1 - 29 ) ;$$

$$( A 1 - 19 ) + ( A 1 - 31 ) ; ( A 1 - 19 ) + ( A 1 - 39 ) ; ( A 1 - 19 ) + ( A 1 - 41 ) ;$$

$$( A 1 - 19 ) + ( A 1 - 83 ) ; ( A 1 - 19 ) + ( A 1 - 87 ) ; ( A 1 - 19 ) + ( A 2 - 2 ) ;$$

$$( A 1 - 19 ) + ( A 2 - 3 ) ; ( A 1 - 19 ) + ( A 3 - 3 ) ; ( A 1 - 19 ) + ( A 3 - 5 ) ;$$

$$( A 1 - 19 ) + ( A 3 - 7 ) ; ( A 1 - 19 ) + ( A 4 - 1 ) ; ( A 1 - 19 ) + ( A 4 - 2 ) ;$$

$$( A 1 - 19 ) + ( A 4 - 3 ) ; ( A 1 - 20 ) + ( A 1 - 28 ) ; ( A 1 - 20 ) + ( A 1 - 29 ) ;$$

$$( A 1 - 20 ) + ( A 1 - 31 ) ; ( A 1 - 20 ) + ( A 1 - 39 ) ; ( A 1 - 20 ) + ( A 1 - 41 ) ;$$

$$( A 1 - 20 ) + ( A 1 - 83 ) ; ( A 1 - 20 ) + ( A 1 - 87 ) ; ( A 1 - 20 ) + ( A 2 - 2 ) ;$$

$$( A 1 - 20 ) + ( A 2 - 3 ) ; ( A 1 - 20 ) + ( A 3 - 3 ) ; ( A 1 - 20 ) + ( A 3 - 5 ) ;$$

$$( A 1 - 20 ) + ( A 3 - 7 ) ; ( A 1 - 20 ) + ( A 4 - 1 ) ; ( A 1 - 20 ) + ( A 4 - 2 ) ;$$

$$( A 1 - 20 ) + ( A 4 - 3 ) ; ( A 1 - 28 ) + ( A 1 - 29 ) ; ( A 1 - 28 ) + ( A 1 - 31 ) ;$$

$$( A 1 - 28 ) + ( A 1 - 39 ) ; ( A 1 - 28 ) + ( A 1 - 41 ) ; ( A 1 - 28 ) + ( A 1 - 83 ) ;$$

$$( A 1 - 28 ) + ( A 1 - 87 ) ; ( A 1 - 28 ) + ( A 2 - 2 ) ; ( A 1 - 28 ) + ( A 2 - 3 ) ;$$

$$( A 1 - 28 ) + ( A 3 - 3 ) ; ( A 1 - 28 ) + ( A 3 - 5 ) ; ( A 1 - 28 ) + ( A 3 - 7 ) ;$$

$$( A 1 - 28 ) + ( A 4 - 1 ) ; ( A 1 - 28 ) + ( A 4 - 2 ) ; ( A 1 - 28 ) + ( A 4 - 3 ) ;$$

$$( A 1 - 29 ) + ( A 1 - 31 ) ; ( A 1 - 29 ) + ( A 1 - 39 ) ; ( A 1 - 29 ) + ( A 1 - 41 ) ;$$

$$( A 1 - 29 ) + ( A 1 - 83 ) ; ( A 1 - 29 ) + ( A 1 - 87 ) ; ( A 1 - 29 ) + ( A 2 - 2 ) ;$$

$$( A 1 - 29 ) + ( A 2 - 3 ) ; ( A 1 - 29 ) + ( A 3 - 3 ) ; ( A 1 - 29 ) + ( A 3 - 5 ) ;$$

$$( A 1 - 29 ) + ( A 3 - 7 ) ; ( A 1 - 29 ) + ( A 4 - 1 ) ; ( A 1 - 29 ) + ( A 4 - 2 ) ;$$

$$( A 1 - 29 ) + ( A 4 - 3 ) ; ( A 1 - 31 ) + ( A 1 - 39 ) ; ( A 1 - 31 ) + ( A 1 - 41 ) ;$$

$$( A 1 - 31 ) + ( A 1 - 83 ) ; ( A 1 - 31 ) + ( A 1 - 87 ) ; ( A 1 - 31 ) + ( A 2 - 2 ) ;$$

$$( A 1 - 31 ) + ( A 2 - 3 ) ; ( A 1 - 31 ) + ( A 3 - 3 ) ; ( A 1 - 31 ) + ( A 3 - 5 ) ;$$

$$( A 1 - 31 ) + ( A 3 - 7 ) ; ( A 1 - 31 ) + ( A 4 - 1 ) ; ( A 1 - 31 ) + ( A 4 - 2 ) ;$$

$$( A 1 - 31 ) + ( A 4 - 3 ) ; ( A 1 - 39 ) + ( A 1 - 41 ) ; ( A 1 - 39 ) + ( A 1 - 83 ) ;$$

$$( A 1 - 39 ) + ( A 1 - 87 ) ; ( A 1 - 39 ) + ( A 2 - 2 ) ; ( A 1 - 39 ) + ( A 2 - 3 ) ;$$

$$( A 1 - 39 ) + ( A 3 - 3 ) ; ( A 1 - 39 ) + ( A 3 - 5 ) ( A 1 - 39 ) + ( A 3 - 7 ) ;$$

$$( A 1 - 39 ) + ( A 4 - 1 ) ; ( A 1 - 39 ) + ( A 4 - 2 ) ; ( A 1 - 39 ) + ( A 4 - 3 ) ;$$

$$( A 1 - 41 ) + ( A 1 - 83 ) ; ( A 1 - 41 ) + ( A 1 - 87 ) ; ( A 1 - 41 ) + ( A 2 - 2 ) ;$$

$$( A 1 - 41 ) + ( A 2 - 3 ) ; ( A 1 - 41 ) + ( A 3 - 3 ) ; ( A 1 - 41 ) + ( A 3 - 5 ) ;$$

$$( A 1 - 41 ) + ( A 3 - 7 ) ; ( A 1 - 41 ) + ( A 4 - 1 ) ; ( A 1 - 41 ) + ( A 4 - 2 ) ;$$

$$( A 1 - 41 ) + ( A 4 - 3 ) ; ( A 1 - 83 ) + ( A 2 - 2 ) ; ( A 1 - 83 ) + ( A 2 - 3 ) ;$$

$$( A 1 - 83 ) + ( A 3 - 3 ) ; ( A 1 - 83 ) + ( A 3 - 5 ) ; ( A 1 - 83 ) + ( A 3 - 7 ) ;$$

$$( A 1 - 83 ) + ( A 4 - 1 ) ; ( A 1 - 83 ) + ( A 4 - 2 ) ; ( A 1 - 83 ) + ( A 4 - 3 ) ;$$

$$( A 1 - 87 ) + ( A 2 - 2 ) ; ( A 1 - 87 ) + ( A 2 - 3 ) ; ( A 1 - 87 ) + ( A 3 - 3 ) ;$$

$$( A 1 - 87 ) + ( A 3 - 5 ) ; ( A 1 - 87 ) + ( A 3 - 7 ) ; ( A 1 - 87 ) + ( A 4 - 1 ) ;$$

$$( A 1 - 87 ) + ( A 4 - 2 ) ; ( A 1 - 87 ) + ( A 4 - 3 ) ; ( A 2 - 2 ) + ( A 2 - 3 ) ;$$

$$( A 2 - 2 ) + ( A 3 - 3 ) ; ( A 2 - 2 ) + ( A 3 - 5 ) ; ( A 2 - 2 ) + ( A 3 - 7 ) ;$$

$$( A 2 - 2 ) + ( A 4 - 1 ) ; ( A 2 - 2 ) + ( A 4 - 2 ) ; ( A 2 - 2 ) + ( A 4 - 3 ) ;$$

$$( A 2 - 3 ) + ( A 3 - 3 ) ; ( A 2 - 3 ) + ( A 3 - 5 ) ; ( A 2 - 3 ) + ( A 3 - 7 ) ;$$

$$( A 2 - 3 ) + ( A 4 - 1 ) ; ( A 2 - 3 ) + ( A 4 - 2 ) ; ( A 2 - 3 ) + ( A 4 - 3 ) ;$$

$$( A 3 - 3 ) + ( A 3 - 5 ) ; ( A 3 - 3 ) + ( A 3 - 7 ) ; ( A 3 - 3 ) + ( A 4 - 1 ) ;$$

$$( A 3 - 3 ) + ( A 4 - 2 ) ; ( A 3 - 3 ) + ( A 4 - 3 ) ; ( A 3 - 5 ) + ( A 3 - 7 ) ;$$

$$( A 3 - 5 ) + ( A 4 - 1 ) ; ( A 3 - 5 ) + ( A 4 - 2 ) ; ( A 3 - 5 ) + ( A 4 - 3 ) ;$$

$$( A 3 - 7 ) + ( A 4 - 1 ) ; ( A 3 - 7 ) + ( A 4 - 2 ) ; ( A 3 - 7 ) + ( A 4 - 3 ) ;$$

$$( A - 1 ) + ( A 4 - 2 ) ; ( A 4 - 1 ) + ( A 4 - 3 ) ; and ( A 4 - 2 ) + ( A 4 - 3 ) .$$

Additionally, the ALS inhibitor herbicides to be used according to the invention may comprise further components, for example agrochemically active compounds of a different type of mode of action and/or the formulation auxiliaries and/or additives customary in crop protection, or may be used together with these.

The ALS inhibitor herbicide(s) to be used according to the invention or combinations of various such ALS inhibitor herbicides may furthermore comprise various agrochemical active compounds, for example from the group of the safeners, fungicides, insecticides, or from the group of the formulation auxiliaries and additives customary in crop protection.

In a further embodiment, the invention relates to the use of effective amounts of ALS inhibitor herbicide(s) (i.e. members of the groups (A), (B) and/or (C)) and non-ALS inhibitor herbicides (i.e. herbicides showing a mode of action that is different to the inhibition of the ALS enzyme [acetohydroxyacid synthase; EC 2.2.1.6](group D herbicides) in order obtain synergistic effect for the control of unwanted vegetation.

Such synergistic actions can be observed, for example, when applying one or more ALS inhibitor herbicides (i.e. members of the groups (A), (B), and/or (C)) and one or more non-ALS inhibitor herbicides (group D herbicides) together, for example as a coformulation or as a tank mix; however, they can also be observed when the active compounds are applied at different times (splitting). It is also possible to apply the ALS inhibitor herbicides and non-ALS inhibitor herbicides in a plurality of portions (sequential application), for example pre-emergence applications followed by post-emergence applications or early post-emergence applications followed by medium or late post-emergence applications. Preference is given here to the joint or almost simultaneous application of the herbicides ((A), (B) and/or (C)) and (D) of the combination in question.

Suitable partner herbicides to be applied together with ALS inhibitor herbicides are, for example, the following herbicides which differ structurally from the herbicides belonging to the groups (A), (B), and (C) as defined above, preferably herbicidally active compounds whose action is based on inhibition of, for example, acetyl coenzyme A carboxylase, PSI, PSII, HPPDO, phytoene desaturase, protoporphyrinogen oxidase, glutamine synthetase, cellulose biosynthesis, 5-enol-pyruvyl-shikimate 3-phosphate synthetase, as described, for example, in Weed Research 26, 441-445 (1986), or “The Pesticide Manual”, 14th edition, The British Crop Protection Council, 2007, or 15th edition 2010, or in the corresponding “ePesticide Manual”, Version 5 (2010), in each case published by the British Crop Protection Council, (hereinbelow in short also “PM”), and in the literature cited therein. Lists of common names are also available in “The Compendium of Pesticide Common Names” on the internet. Herbicides known from the literature (in brackets behind the common name hereinafter also classified by the indicators D1 to D426), which can be combined with ALS-inhibitor herbicides of groups (A), (B) and/or (C) and to be used according to present invention are, for example, the active compounds listed below: (note: the herbicides are referred to either by the “common name” in accordance with the International Organization for Standardization (ISO) or by the chemical name, together where appropriate with a customary code number, and in each case include all use forms, such as acids, salts, esters and isomers, such as stereoisomers and optical isomers, in particular the commercial form or the commercial forms, unless the context indicates otherwise. The citation given is of one use form and in some cases of two or more use forms): acetochlor (=D1), acibenzolar (=D2), acibenzolar-S-methyl (=D3), acifluorfen (=D4), acifluorfen-sodium (=D5), aclonifen (=D6), alachlor (=D7), allidochlor (=D8), alloxydim (=D9), alloxydim-sodium (=D10), ametryn (=D11), amicarbazone (=D12), amidochlor (=D13), aminocyclopyrachlor (=D14), aminopyralid (=D15), amitrole (=D16), ammonium sulfamate (=D17), ancymidol (=D18), anilofos (=D19), asulam (=D20), atrazine (=D21), azafenidin (=D22), aziprotryn (=D23), beflubutamid (=D24), benazolin (=D25), benazolin-ethyl (=D26), bencarbazone (=D27), benfluralin (=D28), benfuresate (=D29), bensulide (=D30), bentazone (=D31), benzfendizone (=D32), benzobicyclon (=D33), benzofenap (=D34), benzofluor (=D35), benzoylprop (=D36), bicyclopyrone (=D37), bifenox (=D38), bilanafos (=D39), bilanafos-sodium (=D40), bromacil (=D41), bromobutide (=D42), bromofenoxim (=D43), bromoxynil (=D44), bromuron (=D45), buminafos (=D46), busoxinone (=D47), butachlor (=D48), butafenacil (=D49), butamifos (=D50), butenachlor (=D51), butralin (=D52), butroxydim (=D53), butylate (=D54), cafenstrole (=D55), carbetamide (=D56), carfentrazone (=D57), carfentrazoneethyl (=D58), chlomethoxyfen (=D59), chloramben (=D60), chlorazifop (=D61), chlorazifop-butyl (=D62), chlorbromuron (=D63), chlorbufam (=D64), chlorfenac (=D65), chlorfenac-sodium (=D66), chlorfenprop (=D67), chlorflurenol (=D68), chlorflurenol-methyl (=D69), chloridazon (=D70), chlormequat-chloride (=D71), chlornitrofen (=D72), chlorophthalim (=D73), chlorthal-dimethyl (=D74), chlorotoluron (=D75), cinidon (=D76), cinidon-ethyl (=D77), cinmethylin (=D78), clethodim (=D79), clodinafop (=D80), clodinafop-propargyl (=D81), clofencet (=D82), clomazone (=D83), clomeprop (=D84), cloprop (=D85), clopyralid (=D86), cloransulam (=D87), cloransulam-methyl (=D88), cumyluron (=D89), cyanamide (=D90), cyanazine (=D91), cyclanilide (=D92), cycloate (=D93), cycloxydim (=D94), cycluron (=D95), cyhalofop (=D96), cyhalofop-butyl (=D97), cyperquat (=D98), cyprazine (=D99), cyprazole (=D100), 2,4-D (=D101), 2,4-DB (=D102), daimuron/dymron (=D103), dalapon (=D104), daminozide (=D105), dazomet (=D106), n-decanol (=D-107), desmedipham (=D108), desmetryn (=D109), detosyl-pyrazolate (=D110), diallate (=D111), dicamba (=D112), dichlobenil (=D113), dichlorprop (=D114), dichlorprop-P (=D115), diclofop (=D116), diclofop-methyl (=D117), diclofop-P-methyl (=D118), diethatyl (=D119), diethatyl-ethyl (=D120), difenoxuron (=D121), difenzoquat (=D122), diflufenican (=D123), diflufenzopyr (=D124), diflufenzopyr-sodium (=D125), dimefuron (=D126), dikegulac-sodium (=D127), dimefuron (=D128), dimepiperate (=D129), dimethachlor (=D130), dimethametryn (=D131), dimethenamid (=D132), dimethenamid-P (=D133), dimethipin (=D134), dimetrasulfuron (=D135), dinitramine (=D136), dinoseb (=D137), dinoterb (=D138), diphenamid (=D139), dipropetryn (=D140), diquat (=D141), diquat-dibromide (=D142), dithiopyr (=D143), diuron (=D144), DNOC (=D145), eglinazine-ethyl (=D146), endothall (=D147), EPTC (=D148), esprocarb (=D149), ethalfluralin (=D150), ethephon (=D151), ethidimuron (=D152), ethiozin (=D153), ethofumesate (=D154), ethoxyfen (=D155), ethoxyfen-ethyl (=D156), etobenzanid (=D157), F-5331 (=2-Chlor-4-fluor-5-[4-(3-fluorpropyl)-4, 5-dihydro-5-oxo-1H-tetrazol-1-yl]-phenyl]ethansulfonamid) (=D158), F-7967 (=3-[7-Chlor-5-fluor-2-(trifluormethyl)-1H-10 benzimidazol-4-yl]-1-methyl-6-(trifluormethyl)pyrimidin-2,4(1H,3H)-dion) (=D159), fenoprop (=D160), fenoxaprop (=D161), fenoxaprop-P (=D162), fenoxaprop-ethyl (=D163), fenoxaprop-P-ethyl (=D164), fenoxasulfone (=D165), fentrazamide (=D166), fenuron (=D167), flam prop (=D168), flamprop-M-isopropyl (=D169), flamprop-M-methyl (=D170), fluazifop (=D171), fluazifop-P (=D172), fluazifop-butyl 15 (=D173), fluazifop-P-butyl (=D174), fluazolate (=D175), fluchloralin (=D176), flufenacet (thiafluamide) (=D177), flufenpyr (=D178), flufenpyr-ethyl (=D179), flumetralin (=D180), flumiclorac (=D181), flumiclorac-pentyl (=D182), flumioxazin (=D183), flumipropyn (=D184), fluometuron (=D185), fluorodifen (=D186), fluoroglycofen (=D187), fluoroglycofen-ethyl (=D188), flupoxam (=D189), 20 flupropacil (=D190), flupropanate (=D191), flurenol (=D192), flurenol-butyl (=D193), fluridone (=D194), flurochloridone (=D195), fluroxypyr (=D196), fluroxypyr-meptyl (=D197), flurprimidol (=D198), flurtamone (=D199), fluthiacet (=D200), fluthiacet-methyl (=D201), fluthiamide (=D202), fomesafen (=203), forchlorfenuron (=D204), fosamine (=D205), furyloxyfen (=D206), gibberellic acid 25 (=D207), glufosinate (=D208), glufosinate-ammonium (=D209), glufosinate-P (=D210), glufosinate-P-ammonium (=D211), glufosinate-P-sodium (=D212), glyphosate (=D213), glyphosate-isopropylammonium (=D214), H-9201 (=O-(2,4-Dimethyl-6-nitrophenyl)-O-ethyl-isopropylphosphoramidothioat) (=D215), halosafen (=D216), haloxyfop (=D217), haloxyfop-P (=D218), haloxyfop-ethoxyethy (=D219), haloxyfop-P-ethoxyethyl (=D220), haloxyfop-methyl (=D221), haloxyfopP-methyl (=D222), hexazinone (=D223), HW-02 (=1-(Dimethoxyphosphoryl)-ethyl(2,4-dichlorphenoxy)acetate) (=D224), inabenfide (=D225), indanofan (=D226), indaziflam (=D227), indol-3-acetic acid (IAA) (=D228), 4-indol-3-ylbutyricacid (IBA) (=D229), ioxynil (=D230), ipfencarbazone (=D231), isocarbamid (=D232), isopropalin (=D233), isoproturon (=D234), isouron (=D235), isoxaben (=D236), isoxachlortole (=D237), isoxaflutole (=D238), isoxapyrifop (=D239), KUH-043 (=3-({[5-(Difluormethyl)-1-methyl-3-(trifluormethyl)-1H-pyrazol-4-yl]methyl}sulfonyl)-5,5-dimethyl-4,5-dihydro-1,2-oxazol) (=D240), karbutilate (=D241), ketospiradox (=D242), lactofen (=D243), lenacil (=D244), linuron (=D245), maleic hydrazide (=D246), MCPA (=D247), MCPB (=D248), MCPB-methyl, -ethyl and -sodium (=D249), mecoprop (=D250), mecoprop-sodium (=D251), mecoprop-butotyl (=D252), mecoprop-P-butotyl (=D253), mecoprop-P-dimethylammonium (=D254), mecoprop-P-2-ethylhexyl (=D255), mecoprop-P-potassium (=D256), mefenacet (=D257), mefluidide (=D258), mepiquat-chloride (=D259), mesotrione (=D260), methabenzthiazuron (=D261), metam (=D262), metamifop (=D263), metamitron (=D264), metazachlor (=D265), metazole (=D266), methiopyrsulfuron (=D267), methiozolin (=D268), methoxyphenone (=D269), methyldymron (=D270), 1-methylcyclopropen (=D271), methylisothiocyanat (=D272), metobenzuron (=D273), metobromuron (=D274), metolachlor (=D275), S-metolachlor (=D-276), metoxuron (=D277), metribuzin (=D278), molinate (=D279), monalide (=D280), monocarbamide (=D281), monocarbamide-dihydrogensulfate (=D282), monolinuron (=D283), monosulfuronester (=D284), monuron (=D285), MT-128 (=6-Chlor-N-[(2E)-3-chlorprop-2-en-1-yl]-5-methyl-N-phenylpyridazin-3-amine) (=D286), MT-5950 (=N-[3-Chlor-4-(1-methylethyl)-phenyl]-2-methylpentanamide) (=D287), NGGC-011 (=D288), naproanilide (=D289), napropamide (=D290), naptalam (=D291), NC-310 (=4-(2,4-Dichlorobenzoyl)-1-methyl-5-benzyloxypyrazole) (=D292), neburon (=D293), nipyraclofen (=D294), nitralin (=D295), nitrofen (=D296), nitrophenolat-sodium (isomer mixture) (=D297), nitrofluorfen (=D298), nonanoic acid (=D299), norflurazon (=D300), orbencarb (=D301), oryzalin (=D302), oxadiargyl (=D303), oxadiazon (=D304), oxaziclomefone (=D305), oxyfluorfen (=D306), paclobutrazol (=D307), paraquat (=D308), paraquat-dichloride (=D309), pelargonic acid (nonanoic acid) (=D310), pendimethalin (=D311), pendralin (=D312), pentanochlor (=D313), pentoxazone (=D314), perfluidone (=D315), pethoxamid (=D317), phenisopham (=D318), phenmedipham (=D319), phenmedipham-ethyl (=D320), picloram (=D321), picolinafen (=D322), pinoxaden (=D323), piperophos (=D324), pirifenop (=D325), pirifenop-butyl (=D326), pretilachlor (=D327), probenazole (=D328), profluazol (=D329), procyazine (=D330), prodiamine (=D331), prifluraline (=D332), profoxydim (=D333), prohexadione (=D334), prohexadionecalcium (=D335), prohydrojasmone (=D336), prometon (=D337), prometryn (=D338), propachlor (=D339), propanil (=D340), propaquizafop (=D341), propazine (=D342), propham (=D343), propisochlor (=D344), propyzamide (=D345), prosulfalin (=D346), prosulfocarb (=D347), prynachlor (=D348), pyraclonil (=D349), pyraflufen (=D350), pyraflufen-ethyl (=D351), pyrasulfotole (=D352), pyrazolynate (pyrazolate) (=D353), pyrazoxyfen (=D354), pyribambenz (=D355), pyributicarb (=D356), pyridafol (=D357), pyridate (=D358), pyriminobac (=D359), 15 pyrimisulfan (=D360), pyroxasulfone (=D361), quinclorac (=D362), quinmerac (=D363), quinoclamine (=D364), quizalofop (=D365), quizalofop-ethyl (=D366), quizalofop-P (=D367), quizalofop-P-ethyl (=D368), quizalofop-P-tefuryl (=D369), saflufenacil (=D370), secbumeton (=D371), sethoxydim (=D372), siduron (=D373), simazine (=D374), simetryn (=D375), SN-106279 (=Methyl-(2R)-2-({7-20 [2-chlor-4-(trifluormethyl)phenoxy]-2-naphthyl}oxy)-propanoate) (=D376), sulcotrione (=D377), sulfallate (CDEC) (=D378), sulfentrazone (=D379), sulfosate (glyphosate-trimesium) (=D380), SYN-523 (=D381), SYP-249 (=1-Ethoxy-3-methyl-1-oxobut-3-en-2-yl-5-[2-chlor-4-(trifluormethyl)phenoxy]-2-nitrobenzoate) (=D382), tebutam (=D383), tebuthiuron (=D384), teenazene (=D385), tefuryltrione (=D386), tembotrione (=D387), tepraloxydim (=D388), terbacil (=D389), terbucarb (=D390), terbuchlor (=D391), terbumeton (=D392), terbuthylazine (=D393), terbutryn (=D394), thenylchlor (=D395), thiafluamide (=D396), thiazafluron (=D397), thiazopyr (=D398), thidiazimin (=D399), thidiazuron (=D400), thiobencarb (=D401), tiocarbazil (=D402), topramezone (=D403), tralkoxydim (=D404), triallate (=D405), triaziflam (=D406), triazofenamide (=D407), trichloracetic acid (TCA) (=D408), triclopyr (=D409), tridiphane (=D410), trietazine (=D411), trifluralin (=D412), trimeturon (=D413), trinexapac (=D414), trinexapac-ethyl (=D415), tsitodef (=D416), uniconazole (=D417), uniconazole-P (=D418), vernolate (=D419), ZJ-0862 (=3,4-Dichlor-N-{2-[(4,6-dimethoxypyrimidin-2-yl)oxy]benzyl}aniline) (=D420), and the below compounds defined by their chemical structure, respectively

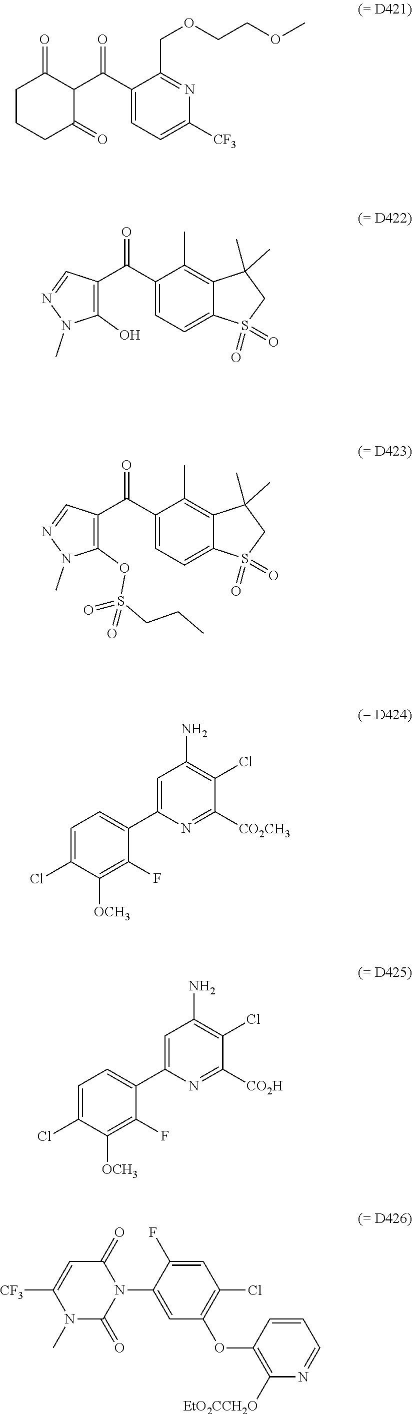

Preferably, further herbicides which differ structurally and via their mode of action from the ALS inhibitor herbicides belonging to the groups (A), (B), and (C) as defined above and to be applied according to the present invention for control of unwanted vegetation in ALS inhibitor herbicide tolerant hybrid Beta vulgaris plants, preferably sugar beet plants as herein described.

In connection with ALS inhibitor herbicides belonging to the groups (A), (B), and (C) are those belonging to the group of: chloridazon (=D70), clethodim (=D79), clodinafop (=D80), clodinafop-propargyl (=D81), clopyralid (=D86), cycloxydim (=D94), desmedipham (=D108), dimethenamid (=D132), dimethenamid-P (=D133), ethofumesate (=D154), fenoxaprop (=D161), fenoxaprop-P (=D162), fenoxaprop-ethyl (=D163), fenoxaprop-P-ethyl (=D164), fluazifop (=D171), fluazifop-P (=D172), fluazifopbutyl (=D173), fluazifop-P-butyl (=D174), glufosinate (=D208), glufosinateammonium (=D209), glufosinate-P (=D210), glufosinate-P-ammonium (=D211), glufosinate-P-sodium (=D212), glyphosate (=D213), glyphosate-isopropylammonium (=D214), haloxyfop (=D217), haloxyfop-P (=D218), haloxyfopethoxyethyl (=D219), haloxyfop-P-ethoxyethyl (=D220), haloxyfop-methyl (=D221), haloxyfop-P-methyl (=D222), lenacil (=D244), metamitron (=D264), phenmedipham (=D319), phenmedipham-ethyl (=D320), propaquizafop (=D341), quinmerac (=D363), quizalofop (=D365), quizalofop-ethyl (=D366), quizalofop-P (=D367), quizalofop-P-ethyl (=D368), quizalofop-P-tefuryl (=D369), sethoxydim (=D372)

Even more preferably, further herbicides which differ from the ALS inhibitor herbicides belonging to the groups (A), (B), and (C) as defined above and to be applied according to the invention in connection with ALS inhibitor herbicides belonging to the groups (A), (B), and (C) are those belonging to the group of: desmedipham (=D108), ethofumesate (=D154), glufosinate (=D208), glufosinateammonium (=D209), glufosinate-P (=D210), glufosinate-P-ammonium (=D211), glufosinate-P-sodium (=D212), glyphosate (=D213), glyphosate-isopropylammonium (=D214), lenacil (=D244), metamitron (=D264), phenmedipham (=D319), phenmedipham-ethyl (=D320).

Mixtures containing ALS inhibitor herbicides and non-ALS inhibitor herbicides, compositions comprising mixtures of one or more ALS inhibitor herbicide(s) (compounds belonging to one or more of groups (A), (B) and (C)) and non-ALS inhibitor heribicide(s) (group (D) members; as defined above) that are of very particular interest in order to be used according to present invention for control of unwanted vegetation are:

$$( A 1 - 1 ) + ( D 108 ) ; ( A 1 - 1 ) + ( D 154 ) ; ( A 1 - 1 ) + ( D 208 ) ;$$

$$( A 1 - 1 ) + ( D 209 ) ; ( A 1 - 1 ) + ( D 210 ) ; ( A 1 - 1 ) + ( D 212 ) ;$$

$$( A 1 - 1 ) + ( D 213 ) ; ( A 1 - 1 ) + ( D 214 ) ; ( A 1 - 1 ) + ( D 244 ) ;$$

$$( A 1 - 1 ) + ( D 264 ) ; ( A 1 - 1 ) + ( D 319 ) ; ( A 1 - 1 ) + ( D 320 ) .$$

$$( A 1 - 13 ) + ( D 108 ) ; ( A 1 - 13 ) + ( D 154 ) ; ( A 1 - 13 ) + ( D 208 ) ;$$

$$( A 1 - 13 ) + ( D 209 ) ; ( A 1 - 13 ) + ( D 210 ) ; ( A 1 - 13 ) + ( D 212 ) ;$$

$$( A 1 - 13 ) + ( D 213 ) ; ( A 1 - 13 ) + ( D 214 ) ; ( A 1 - 13 ) + ( D 244 ) ;$$

$$( A 1 - 13 ) + ( D 264 ) ; ( A 1 - 13 ) + ( D 319 ) ; ( A 1 - 13 ) + ( D 320 ) .$$

$$( A 1 - 16 ) + ( D 108 ) ; ( A 1 - 16 ) + ( D 154 ) ; ( A 1 - 16 ) + ( D 208 ) ;$$

$$( A 1 - 16 ) + ( D 209 ) ; ( A 1 - 16 ) + ( D 210 ) ; ( A 1 - 16 ) + ( D 212 ) ;$$

$$( A 1 - 16 ) + ( D 216 ) ; ( A 1 - 16 ) + ( D 214 ) ; ( A 1 - 16 ) + ( D 244 ) ;$$

$$( A 1 - 16 ) + ( D 264 ) ; ( A 1 - 16 ) + ( D 319 ) ; ( A 1 - 16 ) + ( D 320 ) .$$

$$( A 1 - 39 ) + ( D 108 ) ; ( A 1 - 39 ) + ( D 154 ) ; ( A 1 - 39 ) + ( D 208 ) ;$$

$$( A 1 - 39 ) + ( D 209 ) ; ( A 1 - 39 ) + ( D 210 ) ; ( A 1 - 39 ) + ( D 212 ) ;$$

$$( A 1 - 39 ) + ( D 239 ) ; ( A 1 - 39 ) + ( D 214 ) ; ( A 1 - 39 ) + ( D 244 ) ;$$

$$( A 1 - 39 ) + ( D 264 ) ; ( A 1 - 39 ) + ( D 319 ) ; ( A 1 - 39 ) + ( D 320 ) .$$

$$( A 1 - 41 ) + ( D 108 ) ; ( A 1 - 41 ) + ( D 154 ) ; ( A 1 - 41 ) + ( D 208 ) ;$$

$$( A 1 - 41 ) + ( D 209 ) ; ( A 1 - 41 ) + ( D 210 ) ; ( A 1 - 41 ) + ( D 212 ) ;$$

$$( A 1 - 41 ) + ( D 213 ) ; ( A 1 - 41 ) + ( D 214 ) ; ( A 1 - 41 ) + ( D 244 ) ;$$

$$( A 1 - 41 ) + ( D 264 ) ; ( A 1 - 41 ) + ( D 319 ) ; ( A 1 - 41 ) + ( D 320 ) ;$$

$$( A 1 - 83 ) + ( D 108 ) ; ( A 1 - 83 ) + ( D 154 ) ; ( A 1 - 83 ) + ( D 208 ) ;$$

$$( A 1 - 83 ) + ( D 290 ) ; ( A 1 - 83 ) + ( D 210 ) ; ( A 1 - 83 ) + ( D 212 ) ;$$

$$( A 1 - 83 ) + ( D 213 ) ; ( A 1 - 83 ) + ( D 214 ) ; ( A 1 - 83 ) + ( D 244 ) ;$$

$$( A 1 - 83 ) + ( D 264 ) ; ( A 1 - 83 ) + ( D 319 ) ; ( A 1 - 83 ) + ( D 320 ) ;$$

$$( A 1 - 87 ) + ( D 108 ) ; ( A 1 - 87 ) + ( D 154 ) ; ( A 1 - 87 ) + ( D 208 ) ;$$

$$( A 1 - 87 ) + ( D 209 ) ; ( A 1 - 87 ) + ( D 210 ) ; ( A 1 - 87 ) + ( D 212 ) ;$$

$$( A 1 - 87 ) + ( D 213 ) ; ( A 1 - 87 ) + ( D 214 ) ; ( A 1 - 87 ) + ( D 244 ) ;$$

$$( A 1 - 87 ) + ( D 264 ) ; ( A 1 - 87 ) + ( D 319 ) ; ( A 1 - 87 ) + ( D 320 ) .$$

$$( A 2 - 3 ) + ( D 108 ) ; ( A 2 - 3 ) + ( D 154 ) ; ( A 2 - 3 ) + ( D 208 ) ;$$

$$( A 2 - 3 ) + ( D 209 ) ; ( A 2 - 3 ) + ( D 21 \; 0 ) ; ( A 2 - 3 ) + ( D 212 ) ;$$

$$( A 2 - 3 ) + ( D 213 ) ; ( A 2 - 3 ) + ( D 214 ) ; ( A 2 - 3 ) + ( D 244 ) ;$$

$$( A 2 - 3 ) + ( D 264 ) ; ( A 2 - 3 ) + ( D 319 ) ; ( A 2 - 3 ) + ( D 320 ) .$$

$$( B 1 - 2 ) + ( D 108 ) ; ( B 1 - 2 ) + ( D 154 ) ; ( B 1 - 2 ) + ( D 208 ) ;$$

$$( B 1 - 2 ) + ( D 209 ) ; ( B 1 - 2 ) + ( D 21 \; 0 ) ; ( B 1 - 2 ) + ( D 212 ) ;$$

$$( B 1 - 2 ) + ( D 213 ) ; ( B 1 - 2 ) + ( D 214 ) ; ( B 1 - 2 ) + ( D 244 ) ;$$

$$( B 1 - 2 ) + ( D 264 ) ; ( B 1 - 2 ) + ( D 319 ) ; ( B 1 - 2 ) + ( D 320 ) .$$

$$( C 1 - 1 ) + ( D 108 ) ; ( C 1 - 1 ) + ( D 154 ) ; ( C 1 - 1 ) + ( D 208 ) ;$$

$$( C 1 - 1 ) + ( D 209 ) ; ( C 1 - 1 ) + ( D 210 ) ; ( C 1 - 1 ) + ( D 212 ) ;$$

$$( C 1 - 1 ) + ( D 213 ) ; ( C 1 - 1 ) + ( D 214 ) ; ( C 1 - 1 ) + ( D 244 ) ;$$

$$( C 1 - 1 ) + ( D 264 ) ; ( C 1 - 1 ) + ( D 319 ) ; ( C 1 - 1 ) + ( D 320 ) .$$

The application of ALS inhibitor herbicides also act efficiently on perennial weeds which produce shoots from rhizomes, root stocks and other perennial organs and which are difficult to control. Here, the substances can be applied, for example, by the pre-sowing method, the pre-emergence method or the post-emergence method, for example jointly or separately. Preference is given, for example, to application by the post-emergence method, in particular to the emerged harmful plants.

Specific examples may be mentioned of some representatives of the monocotyledonous and dicotyledonous weed flora which can be controlled by the ALS inhibitor herbicides, without the enumeration being restricted to certain species.

Examples of weed species on which the application according to present invention act efficiently are, from amongst the monocotyledonous weed species, Avena spp., Alopecurus spp., Apera spp., Brachiaria spp., Bromus spp., Digitaria spp., Lolium spp., Echinochloa spp., Panicum spp., Phalaris spp., Paa spp., Setaria spp. And also Cyperus species from the annual group, and, among the perennial species, Agropyron, Cynodon, Imperata and Sorghum and also perennial Cyperus species.

In the case of the dicotyledonous weed species, the spectrum of action extends to genera such as, for example, Abutilon spp., Amaranthus spp., Chenopodium spp., Chrysanthemum spp., Galium spp., Ipomoea spp., Kochia spp., Lamium spp., Matricaria spp., Pharbitis spp., Polygonum spp., Sida spp., Sinapis spp., Solanum spp., Stellaria spp., Veronica spp. and Viola spp., Xanthium spp., among the annuals, and Convolvulus, Cirsium, Rumex and Artemisia in the case of the perennial weeds.

As used herein unless clearly indicated otherwise, the term “plant” is intended to mean a plant at any developmental stage.

The present invention furthermore provides a method for controlling unwanted vegetation in Beta vulgaris plants as herein described, preferably in sugar beet, which comprises applying one or more ALS inhibitor herbicides belonging to groups (A), (B) and/or (C) to the plants (for example harmful plants, such as monocotyledonous or dicotyledonous weeds or unwanted crop plants), the seed (seeds or vegetative propagation organs, such as tubers or shoot parts) or to the area in which the plants grow (for example the area under cultivation), for example together or separately.

The present invention furthermore provides a method for controlling unwanted vegetation in Beta vulgaris plants as herein described, preferably in sugar beet, which comprises applying one or more ALS inhibitor herbicide(s) belonging to groups (A), (B) and/or (C) alone or in combination with non-ALS inhibitor herbicides belonging to class (D) compound according to the invention to the plants (for example harmful plants, such as monocotyledonous or dicotyledonous weeds or unwanted crop plants), the seed (seeds or vegetative propagation organs, such as tubers or shoot parts) or to the area in which the plants grow (for example the area under cultivation), for example together or separately. One or more non-ALS inhibitor herbicides may be applied in combination with one or more ALS inhibitor herbicide(s) before, after or simultaneously with the ALS inhibitor herbicide(s) to the plants, the seed or the area in which the plants grow (for example the area under cultivation).

“Unwanted plants” or “unwanted vegetation” are to be understood as meaning all plants which grow in locations where they are unwanted. This can, for example, be harmful plants (for example monocotyledonous or dicotyledonous weeds or unwanted crop plants).

The herbicide combinations to be used according to the invention can be prepared by known processes, for example as mixed formulations of the individual components, if appropriate with further active compounds, additives and/or customary formulation auxiliaries, which combinations are then applied in a customary manner diluted with water, or as tank mixes by joint dilution of the components, formulated separately or formulated partially separately, with water. Also possible is the split application of the separately formulated or partially separately formulated individual components.

It is also possible to apply ALS inhibitor herbicides or the combination comprising ALS inhibitor herbicide(s) and non-ALS inhibitor herbicide(s) in a plurality of portions (sequential application) using, for example, pre-emergence applications followed by post-emergence applications or using early post-emergence applications followed by medium or late post-emergence applications. Preference is given here to the joint or almost simultaneous application of the active compounds of the combination in question.

The herbicides belonging to any of the above defined groups (A), (B), (C) and (D) and to be applied according to present invention can be converted jointly or separately into customary formulations, such as solutions, emulsions suspensions, powders, foams, pastes, granules, aerosols, natural and synthetic materials impregnated with active compound and micro-encapsulations in polymeric materials. The formulations may comprise the customary auxiliaries and additives.

These formulations are produced in a known manner, for example by mixing the active compounds with extenders, that is liquid solvents, pressurized liquefied gases and/or solid carriers, if appropriate with the use of surfactants, that is emulsifiers and/or dispersants, and/or foam formers.

If the extender used is water, it is also possible to use, for example, organic solvents as auxiliary solvents. Suitable liquid solvents are essentially: aromatics, such as xylene, toluene, alkylnaphthalenes, chlorinated aromatics or chlorinated aliphatic hydrocarbons, such as chlorobenzenes, chloroethylenes, or methylene chloride, aliphatic hydrocarbons, such as cyclohexane or paraffins, for example mineral oil fractions, mineral and vegetable oils, alcohols, such as butanol or glycol, and ethers and esters thereof, ketones, such as acetone, methyl ethyl ketone, methyl isobutyl ketone or cyclohexanone, strongly polar solvents, such as dimethylformamide or dimethyl sulfoxide, and also water. Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, 30 alumina and silicates; suitable solid carriers for granules are: for example crushed and fractionated natural rocks, such as calcite, marble, pumice, sepiolite and dolomite, and also synthetic granules of inorganic and organic meals, and granules of organic material, such as sawdust, coconut shells, corn cobs and tobacco stalks; suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and also protein hydrolysates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, and also natural phospholipids, such as cephalins and lecithins and synthetic phospholipids, can be used in the formulations. Other possible additives are mineral and vegetable oils.

The herbicidal action of the herbicide combinations to be used according to the invention can be improved, for example, by surfactants, preferably by wetting agents from the group of the fatty alcohol polyglycol ethers. The fatty alcohol polyglycol ethers preferably comprise 10-18 carbon atoms in the fatty alcohol radical and 2-20 ethylene oxide units in the polyglycol ether moiety. The fatty alcohol polyglycol ethers may be present in nonionic form, or ionic form, for example in the form of fatty alcohol polyglycol ether sulfates, which may be used, for example, as alkali metal salts (for example sodium salts and potassium salts) or ammonium salts, or even as alkaline earth metal salts, such as magnesium salts, such as C12/C14-fatty alcohol diglycol ether sulfate sodium (Genapol® LRO, Clariant GmbH); see, for example, EP-A-0476555, EP-A-0048436, EP-A-0336151 or U.S. Pat. No. 4,400,196 and also Proc. EWRS Symp. “Factors Affecting Herbicidal Activity and Selectivity”, 227-232 (1988). Nonionic fatty alcohol polyglycol ethers are, for example, (C10-C1a)-, preferably (C10-C14)-fatty alcohol polyglycol ethers (for example isotridecyl alcohol polyglycol ethers) which comprise, for example, 2-20, preferably 3-15, ethylene oxide units, for example those from the Genapol® X-series, such as Genapol® X-030, Genapol® X-060, Genapol® X-080 or Genapol® X-150 (all from Clariant GmbH).

The present invention further comprises the combination of ALS inhibitor herbicides belonging to any of the groups (A), (B), and (C) according to present invention with the wetting agents mentioned above from the group of the fatty alcohol polyglycol ethers which preferably contain 10-18 carbon atoms in the fatty alcohol radical and 2-20 ethylene oxide units in the polyglycol ether moiety and which may be present in nonionic or ionic form (for example as fatty alcohol polyglycol ether sulfates). Preference is given to C12/C14-fatty alcohol diglycol ether sulfate sodium (Genapol® LRO, Clariant GmbH) and isotridecyl alcohol polyglycol ether having 3-15 ethylene oxide units, for example from the Genapol® X-series, such as Genapol® X-030, Genapol® X-060, Genapol® X-080 and Genapol® X-150 (all from Clariant GmbH).

Furthermore, it is known that fatty alcohol polyglycol ethers, such as nonionic or ionic fatty alcohol polyglycol ethers (for example fatty alcohol polyglycol ether sulfates) are also suitable for use as penetrants and activity enhancers for a number of other herbicides (see, for example, EP-A-0502014).

The herbicidal action of the herbicide combinations according to the invention can also be enhanced by using vegetable oils. The term vegetable oils is to be understood as meaning oils of oleaginous plant species, such as soybean oil, rapeseed oil, corn oil, sunflower oil, cottonseed oil, linseed oil, coconut oil, palm oil, thistle oil or castor oil, in particular rapeseed oil, and also their transesterification products, for example alkyl esters, such as rapeseed oil methyl ester or rapeseed oil ethyl ester.

The vegetable oils are preferably esters of C10-C22, preferably C12-C20-, fatty acids. The C10-C22 fatty acid esters are, for example, esters of unsaturated or saturated C10-C22 fatty acids, in particular those having an even number of carbon atoms, for example erucic acid, lauric acid, palmitic acid and in particular C18-fatty acids, such as stearic acid, oleic acid, linoleic acid or linolenic acid.

Examples of C10-C22 fatty acid esters are esters obtained by reacting glycerol or glycol with the C10-C22 fatty acids contained, for example, in oils of oleaginous plant species, or C1-C20-alkyl-C10-C22 fatty acid esters which can be obtained, for example, by transesterification of the aforementioned glycerol- or glycol-C10-C22 fatty acid esters with C1-C20-alcohols (for example methanol, ethanol, propanol or butanol). The transesterification can be carried out by known methods as described, for example, in Rompp Chemie Lexikon, 9th edition, Volume 2, page 1343, Thieme Verlag Stuttgart.

Preferred C1-C20-alkyl-C10-C22 fatty acid esters are methyl esters, ethyl esters, propyl esters, butyl esters, 2-ethylhexyl esters and dodecyl esters. Preferred glycol- and glycerol-C10-C2rfatty acid esters are the uniform or mixed glycol esters and glycerol esters of C10-C2rfatty acids, in particular fatty acids having an even number of carbon atoms, for example erucic acid, lauric acid, palmitic acid and, in particular, C18-fatty acids, such as stearic acid, oleic acid, linoleic acid or linolenic acid.

In the herbicidal compositions to be used according to the invention, the vegetable oils can be present, for example, in the form of commercially available oil-containing formulation additives, in particular those based on rapeseed oil, such as Hasten® (Victorian Chemical Company, Australia, hereinbelow referred to as Hasten, main ingredient: rapeseed oil ethyl ester), Actirob® B (Novance, France, hereinbelow referred to as ActirobB, main ingredient: rapeseed oil methyl ester), Rako-Binol® (Bayer AG, Germany, hereinbelow referred to as Rako-Binol, main ingredient: rapeseed oil), Renal® (Stefes, Germany, hereinbelow referred to as Renal, vegetable oil ingredient: rapeseed oil methyl ester) or Stefes Mero® (Stefes, Germany, hereinbelow referred to as Mero, main ingredient: rapeseed oil methyl ester).

It is possible to use colorants, such as inorganic pigments, for example iron oxide, titanium oxide, Prussian Blue, and organic dyes, such as alizarin dyes, azo dyes and metal phthalocyanine dyes, and trace nutrients such as salts of iron, manganese, boron, copper, cobalt, molybdenum and zinc.

The formulations to be used according to present invention generally comprise from 0.1 to 95% by weight of active compounds, preferably from 0.5 to 90% by weight.

As such or in their formulations, the ALS inhibitor herbicides belonging to any of the above defined groups (A), (B), and (C) can also be used as a mixture with other agrochemically active compounds, such as known non-ALS inhibitor herbicides, for controlling unwanted vegetation, for example for controlling weeds or for controlling unwanted crop plants, finished formulations or tank mixes, for example, being possible.

The use of a mixture of ALS inhibitor herbicides belonging to any of the above 30 defined groups (A), (B), and (C) with other known active compounds, such as fungicides, insecticides, acaricides, nematicides, safeners, bird repellants, plant nutrients and soil structure improvers is likewise possible.

The ALS inhibitor herbicides belonging to any of the above defined groups (A), (B), (C) can be used as such, in the form of their formulations or in the use forms prepared therefrom by further dilution, such as ready-to-use solutions, suspensions, emulsions, powders, pastes and granules. Application is carried out in a customary manner, for example by watering, spraying, atomizing, broadcasting.

According to the invention, one or more of the ALS inhibitor herbicides belonging to any of the above defined groups (A), (B), and (C) can be applied either alone or in combination with one or more non-ALS inhibitor herbicides belonging to group (D) to the plants (for example harmful plants, such as monocotyledonous or dicotyledonous weeds or unwanted crop plants), the seed (for example grains, seeds or vegetative propagation organs, such as tubers or shoot parts with buds) or the area under cultivation (for example the soil), preferably to the green plants and parts of plants and, if appropriate, additionally the soil. One possible use is the joint application of the active compounds in the form of tank mixes, where the optimally formulated concentrated formulations of the individual active compounds are, together, mixed in a tank with water, and the spray liquor obtained is applied.

### Additional Definitions

The following definitions are provided to better define the present invention and to guide those of ordinary skill in the art in the practice of the present invention. Unless otherwise noted, terms are to be understood according to conventional usage by those of ordinary skill in the relevant art.

As used herein, the term “plant” includes plant cells, plant protoplasts, plant cells of tissue culture from which beet plants can be regenerated, plant calli, plant clumps and plant cells that are intact in plants or parts of plants such as pollen, flowers, seeds, leaves, stems, and the like. Also included is propagation material and harvestable parts such as roots, particularly beet roots.

As used herein, the term “population” means a genetically heterogeneous collection of plants that share a common parental derivation.

As used herein, the terms “variety” and “cultivar” mean a group of similar plants that by their genetic pedigrees and performance can be identified from other varieties within the same species.

As used herein, an “allele” refers to one of two or more alternative forms of a genomic sequence at a given locus on a chromosome.

As used herein, a “marker” means a detectable characteristic that can be used to discriminate between organisms. Examples of such characteristics include, but are not limited to, genetic markers, biochemical markers, metabolites, morphological characteristics, and agronomic characteristics.

As used herein, the term “phenotype” means the detectable characteristics of a cell or organism that can be influenced by gene expression.

As used herein, the term “genotype” means the specific allelic makeup of a plant.

As used herein, “elite” or “cultivated” variety or line means any variety that has resulted from breeding and selection for superior agronomic performance. An “elite plant” refers to a plant belonging to an elite variety or line. Numerous elite varieties are available and known to those of skill in the art of beet breeding. An “elite population” is an assortment of elite individuals or varieties that can be used to represent the state of the art in terms of agronomically superior genotypes of a given crop species, such as beet. Similarly, an “elite germplasm” or elite strain of germplasm is an agronomically superior germplasm.

As used herein, the term “introgressed,” when used in reference to a genetic locus, refers to a genetic locus that has been introduced into a new genetic background, such as through backcrossing. Introgression of a genetic locus can be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the terms “recombinant” or “recombined” in the context of a chromosomal segment refer to recombinant DNA sequences comprising one or more genetic loci in a configuration in which they are not found in nature, for example as a result of a recombination event between homologous chromosomes during meiosis.

As used herein, the term “linked,” when used in the context of nucleic acid markers and/or genomic regions, means that the markers and/or genomic regions are located on the same linkage group or chromosome such that they tend to segregate together at meiosis.

“Sequence identity” and “sequence similarity” can be determined by alignment of r two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters) share at least a certain minimal percentage of sequence identity. These programs use the Needleman and Wunsch global alignment algorithm to align two sequences over their entire length, maximizing the number of matches and minimizing the number of gaps. Generally, the default parameters are used, with a gap creation penalty=10 and gap extension penalty=0.5 (both for nucleotide and protein alignments). For nucleotides the default scoring matrix used is DNAFULL (Henikoff & Henikoff, 1992, PNAS 89, 10915-10919). Sequence alignments and scores for percentage sequence identity may for example be determined using computer programs, such as EMBOSS, as available on the world wide web under ebi.ac.uk/Tools/psa/emboss_needle/. Alternatively, sequence similarity or identity may be determined by searching against databases such as FASTA, BLAST, etc., but hits should be retrieved and aligned pairwise to compare sequence identity. Two nucleic acid sequences have “substantial sequence identity” if the percentage sequence identity is at least 85%, 90%, 95%, 98%, 99% or more (e.g. at least 99.1, 99.2 99.3 99.4, 99.5, 99.6, 99.7, 99.8, 99.9 or more (as determined by Emboss “needle” using default parameters, i.e. gap creation penalty=10, gap extension penalty=0.5, using scoring matrix DNAFULL for nucleic acids). Markers may sometimes exhibit variation, particularly in regions which are not recognized by the probes.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

An “endogenous” gene means a gene of a plant which has not been introduced into the plant by genetic engineering techniques.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

All publications and patents cited in this disclosure are incorporated by reference in their entirety. To the extent the material incorporated by reference contradicts or is inconsistent with this specification, the specification will supersede any such material.

### Deposit Information.

Seeds of the ALS inhibitor tolerant Beta vulgaris donor line comprising the BVals_W569L allele and referred herein as SU-12-1 have been deposited with the NCIMB, Aberdeen, UK, under Number NCIMB 41705 on Mar. 12, 2010.

Seeds of ALS inhibitor tolerant Beta vulgaris hybrid line comprising the BVals_W569L allele and referred herein as SU-12-1 have been deposited with the NCIMB, Aberdeen, UK, under Number NCIMB 43836 on Aug. 4, 2021.

Seeds of ALS inhibitor tolerant Beta vulgaris hybrid line comprising the BVals_W569L allele and referred herein as SU-12-1 have been deposited with the NCIMB, Aberdeen, UK, under Number NCIMB 43837 on Aug. 4, 2021.

Seeds of ALS inhibitor tolerant Beta vulgaris hybrid line comprising the BVals_W569L allele and referred herein as SU-12-1 have been deposited with the NCIMB, Aberdeen, UK, under Number NCIMB 43838 on Aug. 4, 2021.

Throughout the description, reference is made to the following Sequence Listing entries:
SEQ ID NO. 1: nucleotide sequence of marker M1.
SEQ ID NO. 2: nucleotide sequence of marker M2.
SEQ ID NO. 3: nucleotide sequence of marker M3.
SEQ ID NO. 4: nucleotide sequence of marker M4.
SEQ ID NO. 5: nucleotide sequence of marker M5.
SEQ ID NO. 6: nucleotide sequence of marker M6.
SEQ ID NO. 7: nucleotide sequence of marker M7.
SEQ ID NO. 8: nucleotide sequence of marker M8.
SEQ ID NO. 9: nucleotide sequence of marker M9.
SEQ ID NO. 10: nucleotide sequence of marker M10.
SEQ ID NO. 11: nucleotide sequence of marker M11.
SEQ ID NO. 12: nucleotide sequence of marker M12.
SEQ ID NO. 13: nucleotide sequence of marker M13.
SEQ ID NO. 14: nucleotide sequence of marker for the ALS inhibitor herbicide tolerant ALS gene W569L.
SEQ ID NO. 15: nucleotide sequence of the Beta vulgaris ALS inhibitor herbicide tolerant ALS gene W569L.
SEQ ID NO. 16: amino acid sequence encoded by the Beta vulgaris ALS inhibitor herbicide tolerant ALS gene W569L.
SEQ ID NO. 17: nucleotide sequence of the reference Beta vulgaris ALS inhibitor herbicide tolerant ALS gene (wild-type).
SEQ ID NO. 18: amino acid sequence encoded by the reference Beta vulgaris ALS inhibitor herbicide tolerant ALS gene (wild-type).
SEQ ID NO. 19: nucleotide sequence of marker M14.
SEQ ID NO. 20: nucleotide sequence of marker M15.
SEQ ID NO. 21: nucleotide sequence of marker M16.
SEQ ID NO. 22: nucleotide sequence of marker M17.

### EXAMPLES

Identification of Suitable Genetic Recombinants of the Sugar Beet SU-12-1 Trait Donor within Heterotic Sugar Beet Pools.

### Material & Methods

The founder genotype carrying the BvALS_W569L ALS gene allele conferring tolerance to ALS inhibitor herbicides, such as sulfonyl urea herbicides, indicated hereinafter as SU-12-1, was derived by selecting spontaneous ALS mutations in calli cultivated from diploid sugar beet genotype 7T9044 (as, for example, described by Alexander Dovzhenko, PhD Thesis, Title: “Towards plastid transformation in rapeseed (Brassica napus L.) and sugarbeet (Beta vulgaris L.)”, Ludwig-Maximilians-Universitat München, Germany, 2001) to foramsulfuron and selecting cell colonies which can grow in the presence of up to 3×10-6 M foramsulfuron (as described in WO2012049266A1 and WO2012049268A1, both publications herein incorporated by reference). After regeneration of shoots from the selected cell colonies, in the presence of foramsulfuron, sugar beet plants tolerant to ALS inhibitor herbicides were obtained. The genetic resource used as starting material, was not commercially relevant and particularly not adapted to various geographies, including the European market.

The BvALS_W569L allele of founder SU-12-1 was introgressed in both heterotic pools of sugar beet, the monogerm 2MOT and the multigerm 2MUF pools, by backcrossing and both marker assisted, and phenotype assisted selection.

For backcrossing, two strategies were applied:
(a) Emasculation of target genotype flowers and subsequent pollination by genotypes carrying the BvALS_W569L allele. Successful crossings were determined by applying marker assisted selection (MAS) using KASP marker Mals. This method was typically used in the second generation of backcrossing and above (generations BC1 and higher).
(b) For efficient generation of first backcrossing generations (generation BC0), e.g. start of conversion of entire new generations of 2MUF and 2MOT genotypes, open pollination with excess donor pollen and without emasculation was used. Therefore, new (elite) genotypes and BvALS_W569L allele donor genotypes were transplanted in a ratio of 1:10 in an isolation. Seed of the new (elite) genotypes was harvested and treated with the sulfonyl urea herbicide mixture comprising foramsulfuron and thiencarbazone methyl (commercially available as CONVISO ONE®). Only successful crossings between the (elite) genotype and the resistance donor genotype survived and were selected as generation BC0.

BC1 and BC2 seed were used to screen for homologous recombination events between the SU-12-1 genotype and (elite) germplasm in close proximity to the SU-12-1 derived BvALS_W569L allele by applying KASP markers specifically diagnostic for SU-12-1 genetic fragments (see Table 1). The KASP markers used form a distance ladder with the BvALS_W569L allele as center (see FIG. 1).

### Results and Discussion

The BvALS_W569L mediated ALS inhibitor herbicide tolerance in sugar beet requires for optimal (commercially relevant) tolerance, the presence of the favorable ALS inhibitor herbicide tolerance allele in homozygous state in the hybrids, and thus the presence thereof in both hybrid parents.

The BvALS gene is located on chromosome 5 of the sugar beet genome at approx. 38 cM of the genetic map ZRINT1601 which is very close to the centromeric region located from 37.4 to 37.5 cM. In a worst case scenario, the entire chromosome 5 of founder SU-12-1 could be introgressed along with the BvALS_W569 tolerance allele in respectively the 2MOT and 2MUF heterotic parent pools, resulting in a hybrid that is homozygous for the entire chromosome 5. In the (hypothetical) best case scenario, on the other hand, only the DNA base exchange resulting in the BvALS_W569L amino acid exchange would be introgressed in (elite) genotypes of each pool.

For hybrid breeding in general and sugar beet in particular, it is widely believed, but not mechanistically understood though, that high crop performance and yields are reached through high levels of genetic heterozygosity, a phenomenon called heterosis (Birchler et al., 2010 Heterosis. The Plant Cell 22, 2105-2112). Larger areas of homozygosity, or even entire chromosomes, as outlined above would result in drastic drops of hybrid yield. In breeding, this negative effect is referred to as inbreeding depression.

In addition, alleles derived from the founder SU-12-1 located on this chromosome 5 may be unfavorable for growing and commercializing in particular geographies, such as European markets. The larger the introgressed fragments, the higher the chance that the specific unfavourable alleles present in founder are introgressed by linkage to the BvALS_W569L allele, an effect known as linkage-drag.

Both negative effects are minimized by removing as much of the SU-12-1 genetic fragments and replacing it with the respective genomic regions of 2MOT and 2MUF (elite) genotypes. For all chromosomes except chromosome 5, this is possible by marker assisted selection against the presence of SU-12-1 chromosomes or fragments thereof.

Since chromosome 5 of SU-12-1 carries the favorable BvALS_W569L allele, such replacement of donor fragments on chromosome 5 in the progeny is only possible by genetic homologous recombinations between the founder and (elite) genotypes, particularly chromosome 5, resulting in exchange of chromosome 5 fragments derived from the donor/founder genotype by the corresponding fragments of chromosome 5 derived from (elite) genotypes. The known molecular mechanisms of homologous recombination are described e.g. in Sung & Klein (2006) (Mechanism of homologous recombination: Mediators and helicases take on regulatory functions. Nature Reviews Molecular Cell Biology 7, 739-750).

Detection of presence or absence of SU-12-1 derived genetic fragments or (elite) genotype derived genetic fragments is dependent on the presence and knowledge of DNA polymorphisms that allow specific differentiation between (elite) genotype and SU-12-1 founder. Such polymorphic markers, nucleotide sequence thereof and indication of the allele at the variant position (nucleotide position 31, 30 or 101) either of the SU-12-1 genotype or the 2MOT or 2MUF (elite) genotypes are listed in Table 1.

Over a course of seven years, BC1 and BC2 generations of both 2MOT and 2MUF (elite) genotypes crossed with SU-12-1 derived BvALS_W569L allele founders were screened for homologous recombinations as close as possible to the BvALS_W569L allele. A stepwise progress was developed and is schematically shown in FIG. 1. Such recombinants on both pool sides can be used as parents for combination in hybrid sugar beet plants with increased heterosis, and yield potential, particularly sugar yield potential.

The recombinants obtained in recombinant screens are described in detail in table 2 for the 2MUF pool and table 3 for the 2MOT pool. Markers located upstream and downstream the BvALS_W569L allele approximate the location of a homologous recombination. For instance, founder SU-12-1 shows an A-allele at marker M1 approx. 4.2 cM downstream of the BvALS_W569L marker Mals. If a recombinant line shows a G-allele at position of marker M1, a homologous recombination took place upstream this position. The most advanced 2MOT and 2MUF donor lines carry only 0.09 cM and 0.03 cM of SU-12-1 derived genomic fragment.

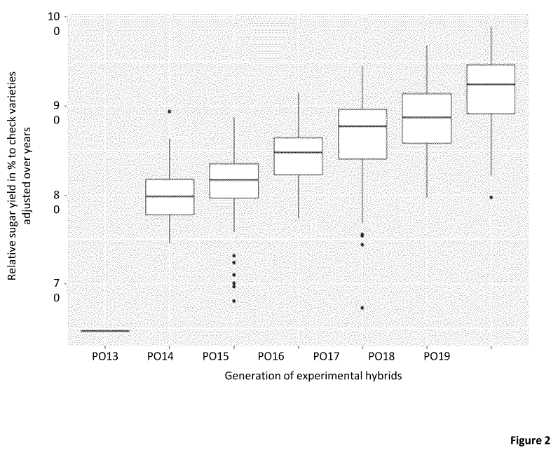

FIG. 2 illustrates the increase in yield performance of hybrid sugar beet plants, obtained by crossing recombinant parent plants from each heterotic pool, with decreasing lengths of SU-12-1 genetic donor fragment size. Relative sugar yield increased were below 70% before starting screens for recombinants (PO_13), increased to 84% with PO_14 hybrids and reached approximately 94% with latest yield data of PO_19.

As will be clear from FIG. 2, both negative effects, linkage-drag and inbreeding depression, are reduced by using advanced recombinant parents.

TABLE 1
Genetic position relative to the Mals marker and nucleotide sequences
of the various markers

| | Distance | | | | |
|---|---|---|---|---|---|
| | from | | | | |
| | Mals | | SU- | MOT/ | SEQ |
| | in | | donor | MUF | ID |
| Marker | CM | Nucleotide sequence | allele | allele | NO |
| M1 | 4.20 | TTTTTTGGAACGGCAACTCCAAACTCCGTTRCTATATGGCCATTTGGTGATGGAACAGCCA | A | G | 1 |
| M2 | 2.91 | TGCAATGTAACTAACACTTGAAATTTGATAYGTACAATTACAGGATTAGAAAATTTCCTGG | C | T | 2 |
| M3 | 0.60 | GTGGGGTTGGTGGAAAGTAAAAAAATGGACRGTTCACATCTTGAATCGATTGTACACAAGG | A | G | 3 |
| M11 | 0.24 | TCGAAGCACTGACTTTGCCTGCCGCCATTAWATCAAATACCAAAAACAACAACAGCCACAA | T | A | 11 |
| M15 | 0.20 | ATTTTAGGAGCAATATTATGTTAACTTTGTAGGTGGCATAGGAGGTGTATGTGGGTTTTTT | G | T | 20 |
| | | TTTCTCACTACCTAGTTTCTTAGTTTTCTAGCTTTATGGKTTGTAACTTAGTTGTTATATG | | | |
| | | CCTATTTATATCAAATTTCCGATCCATTATCTTAAATTTGTATGATATTGCAATGCTTCAR | | | |
| | | GKACTTCTTTGTTGCTTC | | | |
| M14 | 0.05 | GAAGCCATGAATTCAAGAGATGCAACTTTGATTTCTTCCGCTACCATTTTCGGTGTTGTCG | A | C | 19 |
| | | CATCCGCCATTGTTTTTCGCTTCTTCTTCAATAAACCTGMGCAGAAATTTCAGTCTTCGTC | | | |
| | | CAAATTTCCTAAATCAAATGGAGTTCTTCAACGCACAATTTCCGCTACTCGTTCTCCTTTC | | | |
| | | GATCCTTCCAAACGCCAA | | | |
| M4 | 0.02 | GGATGTGCCTCCACCAGAAGGTAAACATCARGCTTTTGTATACTGCTTGGGAGGTCTCCTA | G | A | 4 |
| M5 | 0.01 | CCTATTATCAACGTCAATCACCACCAAAGTYATGAAACTTCAAGATTTTCCAGCCTAGTTG | T | C | 5 |
| Mals | 0.00 | ACAATCAACATTTAGGTATGGTTGTCCAATKGGAAGATAGGTTCTATAAAGCTAACCGGGC | T | G | 14 |
| M12 | 0.02 | ACCTTCTCTGTTTCCGTGATACCATTCTGKAATCCATTTGAACTCCCAACGTTAAATGCT | G | T | 12 |
| M6 | 0.09 | TATCGGAGGTTTTGTCTTTGCTTGCTGTGGYCGAGGTGAAATGTTTTTCGAAAGATTAAAT | T | C | 6 |
| M16 | 0.17 | GAATTGGATCTTGATTCACTCTCAAACACAAAGTAATTGAGCAATCAATTTTAAATCTCA | A | G | 21 |
| | | ACCACAAAGTTATTGATTCAAGGTTTAGAAATCACAAAAARTCCGAAAGTTTTTCACAAA | | | |
| | | GAAAATTAACTATCTAACAGTTTCTTGTTTACTCATGAAAAATGCGCCATTTAAATGCCT | | | |
| | | GAGGACTCTCCTAAACCAAGT | | | |
| M17 | 0.42 | TTAAGATGGGATATATGAGATTTAGGTTGGATCGGATGGTGGCATTGCTGAATTTTCAAA | T | C | 22 |
| | | AAGAGTTATAGCAGGTAAATGGATTTGATAAAGTACTTCTYACTTTCATCCGAAGTATGA | | | |
| | | ATCATCAAATCAACTTGCACATATACTAGCTGCTCATATTTTTGGTCAAAATTGCATTAG | | | |
| | | TTTTTTTAAGTAACATTGATA | | | |
| M13 | 0.48 | TAGAAATATTAAGGTTTAAATTAGTGCATCRGCAAACGTGCCCCAAATAACTGCGTAAACT | G | A | 13 |
| M7 | 1.72 | TCAAGGCAGTGTTGGAGAGCTGTTTCACGGMCTTCTTTTTCGTGTTGGGGAAGATTTAAAG | A | C | 7 |
| M8 | 2.50 | CAATATTAATGTACCCTGGGAACATGGTCGMTGTGGTGATCCGGACTATTTTGCCGTCTGG | A | C | 8 |

TABLE 2
Description of recombination events downstream and upstream of the SU-12-1 BvALS_W569L allele in the
2MOT pool of sugar beet. The positions of the markers on chromosome 5 are expressed in cM based on the
ZRINT1601 genetic map and with marker Mals set as 0 cM. The table also lists the alleles of the SU-12-1 founder
as indicated by these markers. The last entry “FINAL” carries the shortest SU-12-1 genomic fragment.
2MOT

| | | | | | | | | | total |
|---|---|---|---|---|---|---|---|---|---|
| | Recombination downstream of BvALS_W569L | | | | Recombination upstream of BvALS_W569L | | | | SU-12-1 |
| Hybrid- | Position [cM] | Position [cM] on | | SU-12- | Position [cM] | Position [cM] on | | SU-12- | fragment |
| Generation | relative to Mals | chromosome 5 | Marker | 1 allele | relative to Mals | chromosome 5 | Marker | 1 allele | length [cM] |
| PO_14 | 4.20 | 33.7507 | M1 | A | 4.30 | 42.3317 | M10 | G | 8.50 |
| PO_15 | 4.20 | 33.7507 | M1 | A | 4.30 | 42.3317 | M10 | G | 8.50 |
| PO_16 | 4.20 | 33.7507 | M1 | A | 4.20 | 42.2000 | M9 | C | 8.40 |
| PO_17 | 4.20 | 33.7507 | M1 | A | 2.50 | 40.4778 | M8 | A | 6.70 |
| PO_18 | 2.91 | 35.0488 | M2 | C | 2.50 | 40.4778 | M8 | A | 5.41 |
| PO_19 | 0.60 | 37.3520 | M3 | A | 1.70 | 39.7002 | M7 | A | 2.30 |
| PO_20 | 0.02 | 37.9290 | M4 | G | 1.70 | 39.7002 | M7 | A | 1.72 |
| FINAL | 0.01 | 37.9425 | M5 | T | 0.08 | 38.0300 | M6 | T | 0.09 |

TABLE 3
Description of recombination events downstream and upstream of the SU-12-1 BvALS_W569L allele in the 2MUF pool of
sugar beet. The positions of the markers on chromosome 5 are expressed in cM based on the ZRINT1601 genetic map and with
marker Mals set as 0 cM. The positions on chromosome 5 refer to positions on ZRINT1601 genetic map. The table also lists the alleles
of the SU-12-1 founder as indicated by these markers. The last entry “FINAL” carries the shortest SU-12-1 genomic fragment.
2MUF

| | | | | | | | | | total |
|---|---|---|---|---|---|---|---|---|---|
| | Recombination downstream of BvALS_W569L | | | | Recombination upstream of BvALS_W569L | | | | SU-12-1 |
| Hybrid- | Position [cM] | Position [cM] on | | SU-12- | Position [cM] | Position [cM] on | | SU-12- | fragment |
| Generation | relative to Mals | chromosome 5 | Marker | 1 allele | relative to Mals | chromosome 5 | Marker | 1 allele | length [cM] |
| PO_14 | 4.20 | 33.7507 | M1 | A | 4.30 | 42.3317 | M10 | G | 8.50 |
| PO_15 | 4.20 | 33.7507 | M1 | A | 4.30 | 42.3317 | M10 | G | 8.50 |
| PO_16 | 4.20 | 33.7507 | M1 | A | 4.30 | 42.3317 | M10 | G | 8.50 |
| PO_17 | 4.20 | 33.7507 | M1 | A | 4.30 | 42.3317 | M10 | G | 8.50 |
| PO_18 | 2.91 | 35.0488 | M2 | C | 0.48 | 38.4022 | M13 | G | 3.39 |
| PO_19 | 0.24 | 37.7101 | M11 | T | 0.47 | 38.4022 | M13 | G | 0.71 |
| PO_20 | 0.24 | 37.7101 | M11 | T | 0.47 | 38.4022 | M13 | G | 0.71 |
| FINAL | 0.01 | 37.9425 | M5 | T | 0.02 | 37.9738 | M12 | G | 0.03 |

TABLE 4
Description of recombination events downstream and upstream of the SU-12-1 BvALS_W569L allele in the
hybrids as deposited with the NCIMB, Aberdeen, UK, under Number NCIMB 43836, NCIMB 43837 or NCIMB 43838
on Aug. 4, 2021. The positions of the markers on chromosome 5 are expressed in cM based on the ZRINT1601
genetic map and with marker Mals set as 0 cM. The positions on chromosome 5 refer to positions on ZRINT1601
genetic map. The table also lists the alleles of the SU-12-1 founder as indicated by these markers.

| | | | | | | | | | total |
|---|---|---|---|---|---|---|---|---|---|
| | Recombination downstream of BvALS_W569L | | | | Recombination upstream of BvALS_W569L | | | | SU-12-1 |
| Hybrid | Position [cM] | Position [cM] on | | SU-12- | Position [cM] | Position [cM] on | | SU-12- | fragment |
| seed | relative to Mals | chromosome 5 | Marker | 1 allele | relative to Mals | chromosome 5 | Marker | 1 allele | length [cM] |
| NCIMB | 0.01 | 37.9425 | M5 | T | 0.17 | 38.1186 | M16 | A | 0.18 |
| 43836 | | | | | | | | | |
| NCIMB | 0.20 | 37.7503 | M15 | G | 0.42 | 38.3701 | M17 | T | 0.62 |
| 43837 | | | | | | | | | |
| NCIMB | 0.05 | 37.9003 | M14 | A | 0.42 | 38.3701 | M17 | T | 0.47 |
| 43838 | | | | | | | | | |

1. A Beta vulgaris hybrid plant, or hybrid seed, or a part thereof, comprising an ALS-herbicide tolerant endogenous ALS gene allele which is homozygously present on chromosome 5 in said Beta vulgaris hybrid plant or seed, said ALS-herbicide tolerant endogenous ALS gene allele encoding an ALS protein comprising leucine at position 569, said Beta vulgaris hybrid plant or seed being obtainable by crossing two parent Beta vulgaris plants from different heterotic pools, wherein each parent plant comprises the ALS-herbicide tolerant endogenous ALS gene allele in homozygous state and wherein the ALS-herbicide tolerant endogenous ALS gene allele in each parent plant is introgressed from the same ALS-herbicide tolerant endogenous ALS gene allele donor plant, characterized in that chromosomal region of chromosome 5 introgressed from said ALS-herbicide tolerant endogenous ALS gene allele donor plant and located upstream and/or downstream of the ALS inhibitor tolerant endogenous ALS gene allele in said parent plant is sufficiently small to avoid or decrease inbred depression and/or increase heterosis and/or increase sugar yield in said Beta vulgaris hybrid plant.

2. The Beta vulgaris hybrid plant, or hybrid seed, of claim 1, wherein the chromosomal region of chromosome 5 introgressed from said ALS-herbicide tolerant endogenous ALS gene allele donor plant and comprising said ALS-herbicide tolerant endogenous ALS gene allele is localized on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M3 (comprising a nucleotide sequence of SEQ ID NO. 3), marker M4 (comprising a nucleotide sequence of SEQ ID NO. 4), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20) and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22) in one of the parent plants and in the other parent plant, said chromosomal region of chromosome 5 introgressed from said ALS-herbicide tolerant endogenous ALS gene allele donor plant and comprising said ALS-herbicide tolerant endogenous ALS gene allele is flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20) and on the other side of the ALS-herbicide tolerant endogenous ALS gene allele is flanked by a marker selected from the group consisting of marker M6, marker M12, marker M13, marker M7, marker M8, marker M9, marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22).

3. The Beta vulgaris hybrid plant or hybrid seed of claim 1, wherein the chromosomal region introgressed from said ALS-herbicide tolerant endogenous ALS gene allele donor plant and comprising said ALS-herbicide tolerant endogenous ALS gene allele of chromosome 5
a. is flanked by marker M1 and marker M10 on both of the chromosomes 5;
b. is flanked by marker M1 and marker M9 on one of the chromosomes 5 and by marker M1 and marker M10 on the other chromosome 5;
c. is flanked by marker M1 and marker M8 on one of the chromosomes 5 and by marker M1 and marker M10 on the other chromosome 5;
d. is flanked by marker M2 and marker M8 on one of the chromosomes 5 and by marker M2 and M13 on the other chromosome 5;
e. is flanked by marker M3 and marker M7 on one of the chromosomes 5 and by marker M11 and M13 on the other chromosome 5;
f. is flanked by marker M4 and marker M7 on one of the chromosomes 5 and by marker M11 and M13 on the other chromosome 5;
g. is flanked by marker M5 and marker M6 on one of the chromosomes 5 and by marker M5 and M12 on the other chromosome 5;
h. is flanked by marker M5 and marker M16 on both of the chromosomes 5;
i. is flanked by marker M14 and marker M17 on both of the chromosomes 5; or
j. is flanked by marker M15 and marker M17 on both of the chromosomes 5.

4. The Beta vulgaris hybrid plant or hybrid seed of claim 1 wherein the markers comprise a nucleotide at the varying position of the markers, as present in the genomic region of chromosome 5 of the parent plants.

5. The Beta vulgaris hybrid plant or hybrid seed of claim 1 wherein said ALS-herbicide tolerant endogenous ALS gene allele encodes an ALS protein comprising leucine at position 569 such as
an ALS-herbicide tolerant endogenous ALS gene allele comprising a nucleotide sequence having at least 90% sequence identity to the nucleotide sequence of SEQ ID NO 15 or encoding an amino acid sequence having at least 90% sequence identity to the nucleotide sequence of SEQ ID NO 16.

6. The Beta vulgaris hybrid plant or hybrid seed of claim 1 wherein the ALS-herbicide tolerant endogenous ALS gene allele donor plant is a Beta vulgaris ALS inhibitor herbicide tolerant plant reference seed of which being deposited as NCIMB 41705.

7. The Beta vulgaris hybrid plant or hybrid seed of claim 1 having a sugar yield of at least 80%, at least 85%, at least 90%, at least 91%, at least 92%, at least 93%, at least 94%, at least 95%, at least 96%, at least 97%, at least 98%, at least 99%, or equal to or greater to the sugar yield of a Beta vulgaris hybrid plant comprising a wild-type allele of the ALS encoding gene on chromosome 5 in homozygous state.

8. A DNA molecule consisting of the chromosomal region of chromosome 5 of a Beta vulgaris ALS inhibitor herbicide tolerant plant comprising an ALS-herbicide tolerant endogenous ALS gene, reference seed of which has been deposited as NCIMB 41705, which is located on a chromosomal interval flanked on one side of the ALS-herbicide tolerant endogenous ALS gene allele by a marker selected from the group consisting of marker M1, marker M2, marker M3, marker M4, marker M11, marker M15, marker M14 and marker M5, and on the other side of the ALS-herbicide tolerant endogenous ALS gene allele is flanked by a marker selected from the group consisting of marker M6, marker M7, marker M8, marker M9 and marker M10, marker M12, marker M13, marker M16 and marker M17.

9. The DNA molecule of claim 8, wherein said chromosomal region is flanked by markers M5 and M6; or is flanked by markers M4 and M7; or is flanked by markers M5 and M12; or is flanked by markers M11 and M13; or is flanked by markers M5 and M16; or is flanked by markers M14 and M17; or is flanked by markers M15 and M17.

10. A Beta vulgaris plant or seed comprising on one or both chromosome 5 a DNA molecule according to claim 8.

11. A method of producing a hybrid Beta vulgaris seed comprising crossing a Beta vulgaris plant according to claim 10 with another Beta vulgaris plant according to claim 10 and harvesting the progeny seed.

12. Use of a hybrid Beta vulgaris plant according to claim 1 for the production of sugar, ethanol, biogas, betaine and/or uridine or for the production of animal feed or for feeding animals.

13. A method for identifying a genomic fragment of chromosome 5 in (elite) a Beta vulgaris plant, introgressed from an ALS-herbicide tolerant Beta vulgaris donor plant, said genomic fragment comprising an ALS-herbicide tolerant endogenous ALS gene allele, or for identifying/selecting a Beta vulgaris plant comprising said genomic fragment of chromosome 5, comprising the steps of
a. identifying the presence of the ALS-herbicide tolerant endogenous ALS gene allele in said plant by a phenotypic or a marker-assisted method; and
b. identifying the presence of at least one allele/nucleotide on a chromosomal interval flanked on one side of the ALS-herbicide tolerant endogenous ALS gene allele by a marker selected from the group consisting of marker M1 (comprising a nucleotide sequence of SEQ ID NO. 1), marker M2 (comprising a nucleotide sequence of SEQ ID NO. 2), marker M5 (comprising a nucleotide sequence of SEQ ID NO. 5), marker M11 (comprising a nucleotide sequence of SEQ ID NO. 11), marker M14 (comprising a nucleotide sequence of SEQ ID NO. 19) and marker M15 (comprising a nucleotide sequence of SEQ ID NO. 20), and on the other side of the ALS-herbicide tolerant endogenous ALS gene allele is flanked by a marker selected from the group consisting of marker M6 (comprising a nucleotide sequence of SEQ ID NO. 6), marker M12 (comprising a nucleotide sequence of SEQ ID NO. 12), marker M13 (comprising a nucleotide sequence of SEQ ID NO. 13), marker M7 (comprising a nucleotide sequence of SEQ ID NO. 7), marker M8 (comprising a nucleotide sequence of SEQ ID NO. 8), marker M9 (comprising a nucleotide sequence of SEQ ID NO. 9), marker M10 (comprising a nucleotide sequence of SEQ ID NO. 10), marker M16 (comprising a nucleotide sequence of SEQ ID NO. 21) and marker M17 (comprising a nucleotide sequence of SEQ ID NO. 22); and
c. optionally selecting an ALS inhibitor tolerant Beta vulgaris plant, wherein said plant exhibits a decreased inbred depression and/or an increased heterosis and/or an increased sugar yield or biomass yield.

14. A DNA molecule comprising the nucleotide sequence of any one of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, or SEQ ID NO. 17.

15. Use of one or more ALS inhibitor herbicide(s) for controlling unwanted vegetation in Beta vulgaris growing areas wherein the Beta vulgaris plants are hybrid Beta vulgaris plants according to claim 1.

16. Use of one or more ALS inhibitor herbicide(s) according to claim 15, wherein the ALS inhibitor herbicide(s) comprises foramsulfuron [CAS RN 173159-57-4](=A1-13) and thiencarbazone-methyl [CAS RN 317815-83-1](=A2-3) or iodosulfuron-methyl-sodium [CAS RN 144550-36-7](=A1-16) and thiencarbazone-methyl [CAS RN 317815-83-1](=A2-3).

17. Use of one or more ALS inhibitor herbicide(s) according to claim 15 in combination with non-ALS inhibitor herbicides (i.e. herbicides showing a mode of action that is different to the inhibition of the ALS enzyme [acetohydroxyacid synthase; EC 2.2.1.6] group D herbicides), and wherein the non-ALS inhibitor herbicide(s) is/are selected form the group consisting of:
chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, glyphosate, glyphosate-isopropylammonium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl, sethoxydim.

18. Method for controlling unwanted vegetation in Beta vulgaris plant growing areas, characterized by:
(a) the presence of Beta vulgaris plants according to claim 1
(b) the application of one or more ALS inhibitor herbicide(s) alone or in combination with one or more herbicide(s) that do(es) not belong to the class of ALS inhibitor herbicides (non-ALS inhibitor herbicides), and
(c) wherein the application of the respective herbicides as defined under (b)
(i) takes place jointly or simultaneously, or
(ii) takes place at different times and/or in a plurality of portions (sequential application), in pre-emergence applications followed by post-emergence applications or early post-emergence applications followed by medium or late post-emergence applications.

19. Method according to claim 18 for controlling unwanted vegetation, and wherein the ALS inhibitor herbicide(s) comprise foramsulfuron [CAS RN 173159-57-4](=A1-13) and thiencarbazone-methyl [CAS RN 317815-83-1](=A2-3) or iodosulfuron-methyl-sodium [CAS RN 144550-36-7](=A1-16) and thiencarbazone-methyl [CAS RN 317815-83-1](=A2-3).

20. Method according to claim 18, and wherein the non-ALS inhibitor herbicide(s) are taken from the group consisting of:
chloridazon, clethodim, clodinafop, clodinafop-propargyl, clopyralid, cycloxydim, desmedipham, dimethenamid, dimethenamid-P, ethofumesate, fenoxaprop, fenoxaprop-P, fenoxaprop-ethyl, fenoxaprop-P-ethyl, fluazifop, fluazifop-P, fluazifop-butyl, fluazifop-P-butyl, glufosinate, glufosinate-ammonium, glufosinate-P, glufosinate-P-ammonium, glufosinate-P-sodium, glyphosate, glyphosate-isopropylammonium, haloxyfop, haloxyfop-P, haloxyfop-ethoxyethyl, haloxyfop-P-ethoxyethyl, haloxyfop-methyl, haloxyfop-P-methyl, lenacil, metamitron, phenmedipham, phenmedipham-ethyl, propaquizafop, quinmerac, quizalofop, quizalofop-ethyl, quizalofop-P, quizalofop-P-ethyl, quizalofop-P-tefuryl, sethoxydim.


SEQUENCE LISTING (22 sequences)

SEQ ID NO: 1 - DNA, 61 bp, synthetic construct
  misc_feature 1..61: note = Nucleotide sequence of marker M1
  source 1..61: mol_type = other DNA
ttttttggaacggcaactccaaactccgttrctatatggccatttggtgatggaacagcca

SEQ ID NO: 2 - DNA, 61 bp, synthetic construct
  misc_feature 1..61: note = Nucleotide sequence of marker M2
  source 1..61: mol_type = other DNA
tgcaatgtaactaacacttgaaatttgataygtacaattacaggattagaaaatttcctgg

SEQ ID NO: 3 - DNA, 61 bp, synthetic construct
  misc_feature 1..61: note = Nucleotide sequence of marker M3
  source 1..61: mol_type = other DNA
gtggggttggtggaaagtaaaaaaatggacrgttcacatcttgaatcgattgtacacaagg

SEQ ID NO: 4 - DNA, 61 bp, synthetic construct
  misc_feature 1..61: note = Nucleotide sequence of marker M4
  source 1..61: mol_type = other DNA
ggatgtgcctccaccagaaggtaaacatcargcttttgtatactgcttgggaggtctccta

SEQ ID NO: 5 - DNA, 61 bp, synthetic construct
  misc_feature 1..61: note = Nucleotide sequence of marker M5
  source 1..61: mol_type = other DNA
cctattatcaacgtcaatcaccaccaaagtyatgaaacttcaagattttccagcctagttg

SEQ ID NO: 6 - DNA, 61 bp, synthetic construct
  misc_feature 1..61: note = Nucleotide sequence of marker M6
  source 1..61: mol_type = other DNA
tatcggaggttttgtctttgcttgctgtggycgaggtgaaatgtttttcgaaagattaaat

SEQ ID NO: 7 - DNA, 61 bp, synthetic construct
  misc_feature 1..61: note = Nucleotide sequence of marker M7
  source 1..61: mol_type = other DNA
tcaaggcagtgttggagagctgtttcacggmcttctttttcgtgttggggaagatttaaag

SEQ ID NO: 8 - DNA, 61 bp, synthetic construct
  misc_feature 1..61: note = Nucleotide sequence of marker M8
  source 1..61: mol_type = other DNA
caatattaatgtaccctgggaacatggtcgmtgtggtgatccggactattttgccgtctgg

SEQ ID NO: 9 - DNA, 61 bp, synthetic construct
  misc_feature 1..61: note = Nucleotide sequence of marker M9
  source 1..61: mol_type = other DNA
gaagttccaattagcaacccggatacgagcmatggacaagatcgaggggggaaaaatcatc

SEQ ID NO: 10 - DNA, 61 bp, synthetic construct
  misc_feature 1..61: note = Nucleotide sequence of marker M10
  source 1..61: mol_type = other DNA
aatatatgctaacctcttgctattaagcaaracgatgatgtcttagatgtaattattccta

SEQ ID NO: 11 - DNA, 61 bp, synthetic construct
  misc_feature 1..61: note = Nucleotide sequence of marker M11
  source 1..61: mol_type = other DNA
tcgaagcactgactttgcctgccgccattawatcaaataccaaaaacaacaacagccacaa

SEQ ID NO: 12 - DNA, 60 bp, synthetic construct
  misc_feature 1..60: note = Nucleotide sequence of marker M12
  source 1..60: mol_type = other DNA
accttctctgtttccgtgataccattctgkaatccatttgaactcccaacgttaaatgct

SEQ ID NO: 13 - DNA, 61 bp, synthetic construct
  misc_feature 1..61: note = Nucleotide sequence of marker M13
  source 1..61: mol_type = other DNA
tagaaatattaaggtttaaattagtgcatcrgcaaacgtgccccaaataactgcgtaaact

SEQ ID NO: 14 - DNA, 61 bp, synthetic construct
  misc_feature 1..61: note = Nucleotide sequence of marker Mals
  source 1..61: mol_type = other DNA
acaatcaacatttaggtatggttgtccaatkggaagataggttctataaagctaaccgggc

SEQ ID NO: 15 - DNA, 1998 bp, Beta vulgaris
  source 1..1998: mol_type = genomic DNA
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaattggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 16 - protein, 665 aa, Beta vulgaris
MAATFTNPTFSPSSTPLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGASMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDDRVTGKLEAFASRAKIVHIDIDSAEIGKNKQPHVSICADVKLALRGMNKILESRIGKLNLDFSKWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQLEDRFYKANRAHTYLGNPSKSADIFPDMLKFAEACDIPSARVSNVADLRAAIQTMLDTPGPYLLDVIVPHQEHVLPMIPSGAGFKDTITEGDGRTSY

SEQ ID NO: 17 - DNA, 1998 bp, Beta vulgaris
  source 1..1998: mol_type = genomic DNA
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 18 - protein, 665 aa, Beta vulgaris
MAATFTNPTFSPSSTPLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGASMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDDRVTGKLEAFASRAKIVHIDIDSAEIGKNKQPHVSICADVKLALRGMNKILESRIGKLNLDFSKWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSKSADIFPDMLKFAEACDIPSARVSNVADLRAAIQTMLDTPGPYLLDVIVPHQEHVLPMIPSGAGFKDTITEGDGRTSY

SEQ ID NO: 19 - DNA, 201 bp, synthetic construct
  misc_feature 1..201: note = Nucleotide sequence of Marker M14
  source 1..201: mol_type = other DNA
gaagccatgaattcaagagatgcaactttgatttcttccgctaccattttcggtgttgtcgcatccgccattgtttttcgcttcttcttcaataaacctgmgcagaaatttcagtcttcgtccaaatttcctaaatcaaatggagttcttcaacgcacaatttccgctactcgttctcctttcgatccttccaaacgccaa

SEQ ID NO: 20 - DNA, 201 bp, synthetic construct
  misc_feature 1..201: note = Nucleotide sequence of Marker M15
  source 1..201: mol_type = other DNA
attttaggagcaatattatgttaactttgtaggtggcataggaggtgtatgtgggtttttttttctcactacctagttttcttagtttctagctttatggkttgtaacttagttgttatatgcctatttatatcaaatttccgatccattatcttaaatttgtatgatattgcaatgcttcargkacttctttgttgcttc

SEQ ID NO: 21 - DNA, 201 bp, synthetic construct
  misc_feature 1..201: note = Nucleotide sequence of Marker M16
  source 1..201: mol_type = other DNA
gaattggatcttgattcactctcaaacacaaagtaattgagcaatcaattttaaatctcaaccacaaagttattgattcaaggtttagaaatcacaaaaartccgaaagtttttcacaaagaaaattaactatctaacagtttcttgtttactcatgaaaaatgcgccatttaaatgcctgaggactctcctaaaccaagt

SEQ ID NO: 22 - DNA, 201 bp, synthetic construct
  misc_feature 1..201: note = Nucleotide sequence of Marker M17
  source 1..201: mol_type = other DNA
ttaagatgggatatatgagatttaggttggatcggatggtggcattgctgaattttcaaaaagagttatagcaggtaaatggatttgataaagtacttctyactttcatccgaagtatgaatcatcaaatcaacttgcacatatactagctgctcatatttttggtcaaaattgcattagtttttttaagtaacattgata